(19) United States
(12) Patent Application Publication
(10) Pub. No.: US 2019/0336490 A1
(43) Pub. Date: Nov. 7, 2019

(54) COMPOSITIONS AND METHODS FOR INCREASING REMYELINATION

(71) Applicant: Frequency Therapeutics, Inc., Woburn, MA (US)

(72) Inventors: Bradley TAIT, North Andover, MA (US); Michael R. JIROUSEK, Chardon, OH (US); Christopher LOOSE, Winchester, MA (US); William W. CHIN, Alexandria, VA (US)

(21) Appl. No.: 16/376,764

(22) Filed: Apr. 5, 2019

Related U.S. Application Data
(60) Provisional application No. 62/660,124, filed on Apr. 19, 2018.
(60) Provisional application No. 62/653,206, filed on Apr. 5, 2018.

(51) Int. Cl. A61K 31/46; A61K 31/40; A61K 31/24; A61K 31/222; A61K 31/4465; A61K 31/501; A61K 31/5377; A61K 31/55; A61K 31/517; A61K 31/4453; A61K 9/00; A61P 21/00

(52) U.S. Cl. CPC A61K 31/46; A61K 31/40; A61K 31/24; A61K 31/222; A61K 31/4465; A61K 31/501; A61P 21/00; A61K 31/55; A61K 31/517; A61K 31/4453; A61K 9/0019; A61K 9/0053; A61K 31/5377

(57) ABSTRACT

Provided are compositions and methods comprising a MAChR antagonist/inverse agonist/partial agonist and a histamine H3 receptor antagonist/inverse agonist/partial agonist for increasing proliferation Oligodendrocyte Progenitor Cells (OPCs) into mature, myelinating oligodendrocytes, and related methods of treating demyelination disorders.

### RELATED APPLICATIONS

This application claims priority to, and the benefit of, U.S. Provisional Application No. 62/653,206 filed on Apr. 5, 2018 and U.S. Provisional Application No. 62/660,124 filed on Apr. 19, 2018, the contents of which are incorporated herein by reference in its entirety.

### FIELD OF THE INVENTION

The present disclosure relates to compositions and methods comprising a muscarinic acetylcholine receptor (MAChR) antagonist/inverse agonist/partial agonist for increasing remyelination in a subject, for example, as standalone agents which have dual activity as MAChR antagonists/inverse agonists/partial agonist and histamine H3 receptor antagonists/inverse agonists/partial agonists, or in combination with separate agents having activity as histamine H3 receptor antagonists/inverse agonists/partial agonists, and related methods of treating demyelinating diseases or conditions.

### BACKGROUND OF THE INVENTION

Myelin is a fatty white substance that surrounds the axon of some nerve cells, forming an electrically insulating layer. The production of the myelin sheath is called myelination or myelinogenesis. Schwann cells myelinate the axons of the peripheral nervous system, and oligodendrocytes, specifically of the interfascicular type, myelinate the axons of the central nervous system. Myelin is essential for the proper functioning of the nervous system.

Demyelination is the loss of the myelin sheath insulating the nerves, and is the hallmark of some neurodegenerative autoimmune diseases, among other conditions. When myelin degrades, conduction of signals along the nerve can be impaired or lost, and the nerve eventually withers. Demyelination results in diverse symptoms determined by the functions of the affected neurons, examples of which include blurry vision, neuropathy, weakness, fatigue, and cognitive impairment, among others.

Research to repair and/or replace damaged myelin sheaths is ongoing. However, there is a need for improved compositions and methods that are able to increase remyelination with minimal side effects. The present disclosure provides these and other advantages.

### SUMMARY OF THE INVENTION

In various aspects the disclosure provides method of increasing oligodendrocyte precursor cell (OPC) differentiation, by contacting an OPC with an agent having dual activity as a muscarinic acetylcholine receptor (MAChR) antagonist/inverse agonist/partial agonist and a histamine H3 receptor antagonist/inverse agonist/partial agonist; or a first agent having activity as a muscarinic acetylcholine receptor (MAChR) antagonist/inverse agonist/partial agonist and a second agent having activity as a histamine H3 receptor antagonist/inverse agonist/partial agonist. The OPC is contacted with the first agent and second agent in any order or simultaneously.

In other aspects the disclosure provides methods of producing an expanded population of oligodendrocytes, by contacting an oligodendrocyte precursor cell (OPC) with an agent having dual activity as a muscarinic acetylcholine receptor (MAChR) antagonist/inverse agonist/partial agonist and a histamine H3 receptor antagonist/inverse agonist/partial agonist or a first agent having activity as a muscarinic acetylcholine receptor (MAChR) antagonist/inverse agonist/partial agonist and a second agent having activity as a histamine H3 receptor antagonist/inverse agonist/partial agonist. The OPC is contacted with the first agent and second agent in any order or simultaneously.

In further aspects the disclosure provides methods of increasing remyelination in a subject in need thereof, by administering to the subject an agent having dual activity as a muscarinic acetylcholine receptor (MAChR) antagonist/inverse agonist/partial agonist and a histamine H3 receptor antagonist/inverse agonist/partial agonist or a first agent having activity as a muscarinic acetylcholine receptor (MAChR) antagonist/inverse agonist/partial agonist and a second agent having activity as a histamine H3 receptor antagonist/inverse agonist/partial agonist The administration of the first agent and second agent can occur in any order or simultaneously,

In yet another aspect the disclosure provides methods of treating a demyelination disease or disorder in a subject in need thereof, by administering to the subject an agent having dual activity as a muscarinic acetylcholine receptor (MAChR) antagonist/inverse agonist/partial agonist and a histamine H3 receptor antagonist/inverse agonist/partial agonist or a first agent having activity as muscarinic acetylcholine receptor (MAChR) antagonist/inverse agonist/partial agonist and a second agent having activity as a histamine H3 receptor antagonist/inverse agonist/partial agonist. The administration of the first agent and second agent can occur in any order or simultaneously.

Also included in the disclosure is a container containing a histamine H3 receptor antagonist/inverse agonist/partial agonist and instructions, where those instructions describe the a histamine H3 receptor antagonist/inverse agonist/partial agonist use in treating or preventing a demyelinating disorder in a subject, wherein the instructions require that the subject has been, or will be, administered MAChR antagonist/inverse agonist/partial agonist and a histamine H3 receptor antagonist/inverse agonist/partial agonist.

Also included in the disclosure is a container containing a MAChR antagonist/inverse agonist/partial agonist and instructions, where those instructions describe the MAChR antagonist/inverse agonist/partial agonist use in treating or preventing a demyelinating disorder in a subject, wherein the instructions require that the subject has been, or will be, administered MAChR antagonist/inverse agonist/partial agonist and a histamine H3 receptor antagonist/inverse agonist/partial agonist.

The subject has a demyelination disease such as Acute disseminated encephalomyelitis (ADEM); Acute hemorrhagic leukoencephalitis; Acute optic neuritis; Acute transverse myelitis; Adrenoleukodystrophy; Adrenomyeloneuropathy; Alexander Disease; Alzheimer's Disease; aminoacidurias; Amyotrophic Lateral Sclerosis; Anti-MAG peripheral neuropathy; Anti-MOG associated spectrum; Balo concentric sclerosis; Brain injury; CAMFAK Syndrome; Canavan Disease; Carbon monoxide toxicity; Central pontine myelinolysis; Cerebral hypoxia; Cerebral ischemia; Charcot-Marie-Tooth disease; Chronic inflammatory demyelinating polyneuropathy; Chronic relapsing inflammatory optic neuritis (CRION); Chronic traumatic encephalopathy; clinically isolated syndrome (CIS); Congenital Cataract; Copper deficiency associated condition; Delayed Post-Hypoxic Leukoencephalopathy; diffuse cerebral sclerosis of Schilder; diffuse myelinoclastic sclerosis; extrapontine myelinolysis; Gaucher disease; Guillain-Barré syndrome; Hereditary neuropathy; hereditary neuropathy with liability to pressure palsy; HTLV-1-associated myelopathy; Hurler syndrome; Hypomyelination; hypoxic brain injury; Krabbe Disease; Leber hereditary optic atrophy and related mitochondrial disorders; leukodystrophic disorders; Marburg multiple sclerosis; Marchiafava-Bignami disease; Metachromatic leukodystrophy; multiple sclerosis; multiple system atrophy; myelinoclastic disorders; myelopathy; nerve injury; neuromyelitis optica; Neuromyelitis optica (NMO); Niemann-Pick disease; optic neuropathy; optic-spinal multiple sclerosis; Osmotic Demyelination Syndrome; Parkinsons; Pelizaeus-Merzbacher Disease; peripheral neuropathy; Phenylketonuria; primary progressive multiple sclerosis (PPMS); progressive inflammatory neuropathy; progressive multifocal leukoencephalopathy; Progressive subcortical ischemic demyelination; progressive-onset multiple sclerosis; relapsing-onset multiple sclerosis; relapsing-remitting multiple sclerosis (RRMS); reperfusion injury; Schilder disease; secondary progressive multiple sclerosis (SPMS); Solitary sclerosis; Spinal Cord Injury; Subacute sclerosing panencephalitis; Tabes dorsalis; Tay-Sachs disease; Traumatic Brain Injury; Tropical spastic paraparesis; Tumefactive multiple sclerosis; or Vitamin B12 deficiency. In preferred embodiments the subject has Multiple Sclerosis, Optic-spinal multiple sclerosis, Amyotrophic Lateral Sclerosis, Chronic relapsing inflammatory optic neuritis (CRION), Neuromyelitis optica, or Chronic inflammatory demyelinating polyneuropathy.

The MAChR antagonist/inverse agonist/partial agonist is for example, Atropine, Benztropine, Chlorpromazine, Clemastine, Dicyclomine, Diphenylpyraline, Disopyramide, Hyoscyamine, Mepenzolate, Orphenadrine, Oxybutynin, Paroxetine, Pentoxyverine, Piperidolate, Propafenone, Propiverine, Quetiapine, Scopolamine, Solifenacin, Tolterodine, Trihexyphenidyl, or Tripelennamine.

The histamine H3 receptor antagonist/inverse agonist/partial agonist is for example A-317920, A-320436, A-331440, ABT-249, ABT-288, ABT-834, AZD-5213, Betahistine, CEP-26401 (Irdabisant), CEP-32215, Ciproxifan, Contilisant, FUB-138, FUB-153, FUB-181, FUB-833, GSK-1004723, GSK-189254, GSK-239512, GSK-247246, GSK-334429, GSK-835726, GT2331, JNJ-31001074/Bavisant, JNJ-39220675, JNJ-5207852, JNJ-6379490, MK-0249, MK-3134, MK-7288, NNC 38-1049, PF-03654746, PF-03654764, Pitolisant (tiprolisant), S 38093, SAR110068, SAR-110894, SUVN G3031, UCL 1390, UW-MD-71, or CAS 1035626-05-1.

In some embodiments the benztropine is at a concentration of about between about 100 nM to 10 μM.

In some embodiments the wherein clemastine is at a concentration of about between about 25 nM to 2.5 μM.

In some embodiments the oxybutynin is at a concentration of about between about 100 nM to 10 μM.

In some embodiments the pentoxyverine is at a concentration of about between about 25 nM to 2.5 μM.

In some embodiments the propiverine is at a concentration of about between about 100 nM to 10 μM.

In some embodiments the, wherein ABT-288 is at a concentration of about between about 10 nM to 1 μM.

In some embodiments the wherein Bavisant is at a concentration of about between about 10 nM to 1 μM.

In some embodiments the wherein GSK239512 is at a concentration of about between about 10 nM to 1 μM.

In some embodiments the wherein Irdabisant is at a concentration of about between about 10 nM to 1 μM.

In some embodiments the wherein MK-0249 is at a concentration of about between about 10 nM to 1 μM.

In some embodiments the pitolisant is at a concentration of about between about 10 nM to 1 μM.

In some embodiments the MAChR antagonist/inverse agonist/partial agonist is administered locally.

In some embodiments the MAChR antagonist/inverse agonist/partial agonist is administered systemically.

In some embodiments the MAChR antagonist/inverse agonist/partial agonist is administered locally and systemically.

In some embodiments the histamine H3 receptor antagonist/inverse agonist/partial agonist is administered locally.

In some embodiments the histamine H3 receptor antagonist/inverse agonist/partial agonist is administered systemically.

In some embodiments the histamine H3 receptor antagonist/inverse agonist/partial agonist is administered locally and systemically.

In some embodiments the local administration is to the CNS via intrathecal such as intracerebroventricular (ICV) or to the eye via intraocular injection or eye drops.

In preferred embodiments the local administration is to CNS via intracerebroventricular (ICV).

In some embodiments the systemic administration is oral or parenteral.

In some embodiments the MAChR antagonist/inverse agonist/partial agonist is benztropine and is administered orally at a dose of 0.1 mg to 100 mg per day.

In some embodiments the MAChR antagonist/inverse agonist/partial agonist is clemastine and is administered orally at a dose of 0.1 mg to 100 mg per day.

In some embodiments the MAChR antagonist/inverse agonist/partial agonist is oxybutynin and is administered orally at a dose of 0.5 mg to 500 mg per day.

In some embodiments the MAChR antagonist/inverse agonist/partial agonist is pentoxyverine, and is administered orally at a dose of 10 mg to 1000 mg per day.

In some embodiments the MAChR antagonist/inverse agonist/partial agonist is propiverine and is administered orally at a dose of 0.5 mg to 500 mg per day.

In some embodiments the wherein histamine H3 receptor antagonist/inverse agonist/partial agonist is ABT-288 and is administered orally at a dose of 0.25 mg to 250 mg per day.

In some embodiments the wherein histamine H3 receptor antagonist/inverse agonist/partial agonist Bavisant and is administered orally at a dose of 0.1 mg to 500 mg per day.

In some embodiments the wherein histamine H3 receptor antagonist/inverse agonist/partial agonist is GSK239512 and is administered orally at a dose of 0.001 mg to 10 mg per day.

In some embodiments the wherein histamine H3 receptor antagonist/inverse agonist/partial agonist is Irdabisant and is administered orally at a dose of 0.01 mg to 50 mg per day.

In some embodiments the, wherein histamine H3 receptor antagonist/inverse agonist/partial agonist is MK-0249 and is administered orally at a dose of 0.1 mg to 100 mg per day.

In some embodiments the wherein histamine H3 receptor antagonist/inverse agonist/partial agonist is pitolisant and is administered orally at a dose of 1 mg to 250 mg per day.

In other aspects the disclosure provides a pharmaceutical composition, comprising a pharmaceutically-acceptable carrier, and an agent having dual activity as a muscarinic acetylcholine receptor (MAChR) antagonist/inverse agonist/partial agonist and a histamine H3 receptor antagonist/inverse agonist/partial agonist; or a first agent having activity as a muscarinic acetylcholine receptor (MAChR) antagonist/inverse agonist/partial agonist and a second agent having activity as a histamine H3 receptor antagonist/inverse agonist/partial agonist.

The MAChR antagonist/inverse agonist/partial agonist is for example, Atropine, Benztropine, Chlorpromazine, Clemastine, Dicyclomine, Diphenylpyraline, Disopyramide, Hyoscyamine, Mepenzolate, Orphenadrine, Oxybutynin, Paroxetine, Pentoxyverine, Piperidolate, Propafenone, Propiverine, Quetiapine, Scopolamine, Solifenacin, Tolterodine, Trihexyphenidyl, or Tripelennamine.

The histamine H3 receptor antagonist/inverse agonist/partial agonist is for example A-317920, A-320436, A-331440, ABT-249, ABT-288, ABT-834, AZD-5213, Betahistine, CEP-26401 (Irdabisant), CEP-32215, Ciproxifan, Contilisant, FUB-138, FUB-153, FUB-181, FUB-833, GSK-1004723, GSK-189254, GSK-239512, GSK-247246, GSK-334429, GSK-835726, GT2331, JNJ-31001074/Bavisant, JNJ-39220675, JNJ-5207852, JNJ-6379490, MK-0249, MK-3134, MK-7288, NNC 38-1049, PF-03654746, PF-03654764, Pitolisant (tiprolisant), S 38093, SAR110068, SAR-110894, SUVN G3031, UCL 1390, UW-MD-71, or CAS 1035626-05-1.

In some embodiments the pharmaceutical composition contains benztropine at a concentration of about between 1 uM to 1000 μM.

In some embodiments the pharmaceutical composition contains clemastine at a concentration of about between 250 nM to 1000 μM.

In some embodiments the pharmaceutical composition contains oxybutynin at a concentration of about between 1 uM to 1000 μM.

In some embodiments the pharmaceutical composition contains pentoxyverine at a concentration of about between 1 uM to 1000 μM.

In some embodiments the pharmaceutical composition contains propiverine is at a concentration of about between 250 nM to 1000 μM.

In some embodiments the pharmaceutical composition contains ABT-288 at a concentration of about between 100 nM to 100 μM.

In some embodiments the pharmaceutical composition contains Bavisant at a concentration of about between 100 nM to 100 μM.

In some embodiments the pharmaceutical composition contains GSK239512 at a concentration of about between 100 nM to 100 μM.

In some embodiments the pharmaceutical composition contains Irdabisant at a concentration of about between 100 nM to 100 μM.

In some embodiments the pharmaceutical composition contains MK-0249 at a concentration of about between 100 nM to 100 μM.

In some embodiments the pharmaceutical composition contains pitolisant at a concentration of about between 100 nM to 100 μM.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTIONS OF THE DRAWINGS

The patent or application file contains at least one drawing executed in color. Copies of this patent or patent application publication with color drawing(s) will be provided by the Office upon request and payment of the necessary fee.

FIG. 1. Illustrative Immunohistochemistry of spines from mice treated with PBS (A), 10 mg/kg clemastine (B) or 10 mg/kg clemastine plus 40 mg/kg pitolisant (C). Histological analyses from sections such as these are shown in D, E, and F.

FIG. 2. Heat map of % MBP positive cells when clemastine is combined with pitolisant. Clemastine concentrations are increased along the y axis and pitolisant concentrations are increased along the x axis. The % MPB positive cells are indicated numerically in each cell of this map, and color coded such that red indicates higher % MBP positive cells and blue indicates lower % MBP positive cells.

FIG. 3. Total number of cells that stained positive for myelin basic protein (MBP) when clemastine was combined with pitolisant. Clemastine concentrations are shown in the x-axis label. Pitolisant concentrations are indicated by bar color.

### DETAILED DESCRIPTION OF THE INVENTION

The invention is based upon the discovery that antagonists/inverse agonists/partial agonists of muscarinic acetylcholine receptor (MAChR) can induce differentiation and/or proliferation of Oligodendrocyte Progenitor Cells (OPCs) into mature, myelinating oligodendrocytes and therefore increase remyelination. However, when administered to a subject MAChRs antagonists/inverse agonists/partial agonists can have dose-limiting side effects such as but for example CNS impairment. It has been discovered that the combination of MAChRs with histamine H3 receptor (H3) antagonists/inverse agonists/partial agonists can induce greater proliferation and/or differentiation of OPCs into mature, myelinating oligodendrocytes compared to either a MAChR antagonist/inverse agonist/partial agonist or a H3 antagonist/inverse agonist/partial agonist alone. Further, it has been discovered that the combination of MAChRs with histamine H3 receptor (H3) antagonists/inverse agonists/partial agonists can induce differentiation of OPCs into oligodendrocytes at reduced concentrations compared to either a MAChR antagonist/inverse agonist/partial agonist or a H3 antagonist/inverse agonist/partial agonist alone. Thus, reducing the undesired and dose-limiting side-effects of the MAChRs antagonists/inverse agonists/partial agonists as well as improving remyelination efficacy.

Accordingly, the present invention provides composition and methods for proliferation and/or differentiation of OPCs into oligodendrocytes using a MAChR antagonist/inverse agonist/partial agonist together with a H3 antagonist/inverse agonist/partial agonist.

Thus, in various aspects the invention provides method of increasing OPC proliferation and/or differentiation; producing an expanded population of OPCs; increasing remyelination and treating demyelination disorders in a subject by contacting a OPCs or administering to the subject MAChR antagonist/inverse agonist/partial agonist together with a H3 antagonist/inverse agonist/partial agonist

### Muscarinic Acetylcholine Receptor (MAChR) Antagonists/Inverse Agonists/Partial Agonists

Muscarinic receptors are characterised through their interaction with muscarine, a water-soluble toxin derived from the mushroom Amanita muscaria that causes substantial activation of the peripheral sympathetic nervous system through its binding to muscarinic AChRs, resulting in convulsions and even death. The muscarinic AChRs occur primarily in the CNS, and are part of a large family of G-protein-coupled receptors (‘G proteins’), which use an intracellular secondary messenger system involving an increase of intracellular calcium to transmit signals inside cells. Binding of acetylcholine to a muscarinic AChR causes a conformational change in the receptor that is responsible for its association with and activation of an intracellular G protein, the latter converting GTP to GDP in order to become activated and dissociate from the receptor. The activated G protein can then act as an enzyme to catalyse downstream intracellular events. Muscarinic receptors are involved in a large number of physiological functions including heart rate and force, contraction of smooth muscles and the release of neurotransmitters. There are five subtypes of muscarinic AChRs based on pharmacological activity: M1-M5. All five are found in the CNS, while M1-M4 are also found in various tissues: M1 AChRs are common in secretory glands; M2 AChRs are found in cardiac tissue; M3 AChRs are found in smooth muscles and in secretion glands. M1, M3 and M5 receptors cause the activation of phospholipase C, generating two secondary messengers (IP3 and DAG) eventually leading to an intracellular increase of calcium, while M2 and M4 inhibit adenylate cyclase, thereby decreasing the production of the second messenger cAMP.

Each muscarinic receptor has unique amino acid sequences located in the amino-terminal (extracellular) region, and in the third intracellular loop, which in some instances allow for selective muscarinic receptor targeting (e.g. M1 targeting)

Thus, in some instances, a MAChR antagonist/inverse agonist/partial agonist reduces or inhibits the activity of a muscarinic receptors in a cell, tissue, or subject, for example, by about or at least about 5, 10, 20, 30, 40, 50, 60, 70, 80, 90, or 100%, relative to a control, for example relative to a baseline level of activity.

In particular embodiments, an agent having activity as MAChR antagonist/inverse agonist/partial agonist has an IC50 in an in vitro MAChR functional assay (for example a OPC Differentiation Assay) ranging from about 0.010-100 μM, 0.10-50 μM, 0.1-40 μM, 0.1-30 μM, 0.1-20 μM, 0.1-10 μM, 0.5-50 μM, 0.5-400 μM, 0.5-30 μM, 0.5-20 μM, 0.5-10 μM, 1.0-100 μM, 1.0-50 μM, 1.0-40 μM, 1.0-30 μM, 1.0-20 μM, or 1.0-10 μM, or about or no more than about 100, 50, 40, 30, 20, 10, 9.0, 8.0, 7.0, 6.0, 5.0, 4.0, 3.0, 2.0, 1.0, 0.9, 0.8, 0.7, 0.6, 0.5, 0.4, 0.3, 0.2, 0.1, or 0.05 μM

In some instances, a MAChR antagonist/inverse agonist/partial agonist has selective activity towards the muscarinic M1 receptor relative to any one or more of the muscarinic M2, M3, M4, and/or M5 receptors. Without being bound by any one theory, in some instances, selective activity towards the M1 receptor improves the undesirable side effects cause by M2, M3, M4, M5 inhibition, including, for example, cardiac reduction in force of contraction and a decrease in the rate of beating (M2), delayed GI emptying (M3), dry mouth (M3), blurred vision (M4), and/or smooth muscle relaxation (M4).

In some instances, a MAChR antagonist/inverse agonist/partial agonist has selective activity towards the muscarinic M1 receptor relative to any one or more of the muscarinic M2, M3, M4, and/or M5 receptors.

In some instances, a MAChR antagonist/inverse agonist/partial agonist has selective activity towards the muscarinic M2 receptor relative to any one or more of the muscarinic M1, M3, M4, and/or M5 receptors.

In some instances, a MAChR antagonist/inverse agonist/partial agonist has selective activity towards the muscarinic M3 receptor relative to any one or more of the muscarinic M1, M2, M4, and/or M5 receptors.

In some instances, a MAChR antagonist/inverse agonist/partial agonist has selective activity towards the muscarinic M4 receptor relative to any one or more of the muscarinic M1, M2, M3, and/or M5 receptors.

In some instances, a MAChR antagonist/inverse agonist/partial agonist has selective activity towards the muscarinic M5 receptor relative to any one or more of the muscarinic M1, M2, M3, and/or M4 receptors.

In some instances, a MAChR antagonist/inverse agonist/partial agonist has selective activity towards the muscarinic M1 receptor the M3 receptor and optionally M5 receptor relative to any one or more of the muscarinic M2 and/or M4 receptors.

By selective activity towards the one muscarinic receptor relative to any one or more of the other muscarinic receptors means that the agent has at least 50%, 55%, 60%, 65%, 70%, 75%, 80%, 85%, 90%, 95%, 100% more activity towards a single muscarinic receptor relative to any one or more of the muscarinic receptors

In some instances, the MAChR antagonist/inverse agonist/partial agonist has activity as a muscarinic receptor antagonist. In some instances, the MAChR antagonist/inverse agonist/partial agonist has activity as a muscarinic inverse agonist. In particular instances, the MAChR antagonist/inverse agonist/partial agonist has activity as a muscarinic partial agonist, for example, a partial agonist that displays antagonist activity in the presence of a full agonist (e.g., endogenous muscarinic receptor ligand). In particular instances, the MAChR antagonist/inverse agonist/partial agonist has activity as a muscarinic competitive antagonist. In some instances, the MAChR antagonist/inverse agonist/partial agonist has activity as a muscarinic non-competitive antagonist. In some embodiments, a MAChR non-competitive antagonist ameliorates or otherwise reduces the undesired CNS effects of anti-muscarinics.

In some instances, the MAChR antagonist/inverse agonist/partial agonist has activity as a muscarinic M1 antagonist. In some instances, the MAChR antagonist/inverse agonist/partial agonist has activity as a muscarinic M1 inverse agonist. In particular instances, the MAChR antagonist/inverse agonist/partial agonist has activity as a muscarinic M1 partial agonist, for example, a partial agonist that displays antagonist activity in the presence of a full agonist (e.g., endogenous muscarinic receptor ligand). In particular instances, the MAChR antagonist/inverse agonist/partial agonist has activity as a muscarinic M1 competitive antagonist. In some instances, the MAChR antagonist/inverse agonist/partial agonist has activity as a muscarinic M1 non-competitive antagonist. In some embodiments, a MAChR non-competitive antagonist ameliorates or otherwise reduces the undesired CNS effects of anti-muscarinics.

In some instances, the MAChR antagonist/inverse agonist/partial agonist has activity as a muscarinic M2 antagonist. In some instances, the MAChR antagonist/inverse agonist/partial agonist has activity as a muscarinic M2 inverse agonist. In particular instances, the MAChR antagonist/inverse agonist/partial agonist has activity as a muscarinic M2 partial agonist, for example, a partial agonist that displays antagonist activity in the presence of a full agonist (e.g., endogenous muscarinic receptor ligand). In particular instances, the MAChR antagonist/inverse agonist/partial agonist has activity as a muscarinic M2 competitive antagonist. In some instances, the MAChR antagonist/inverse agonist/partial agonist has activity as a muscarinic M2 non-competitive antagonist. In some embodiments, a MAChR non-competitive antagonist ameliorates or otherwise reduces the undesired CNS effects of anti-muscarinics.

In some instances, the MAChR antagonist/inverse agonist/partial agonist has activity as a muscarinic M3 antagonist. In some instances, the MAChR antagonist/inverse agonist/partial agonist has activity as a muscarinic M3 inverse agonist. In particular instances, the MAChR antagonist/inverse agonist/partial agonist has activity as a muscarinic M3 partial agonist, for example, a partial agonist that displays antagonist activity in the presence of a full agonist (e.g., endogenous muscarinic receptor ligand). In particular instances, the MAChR antagonist/inverse agonist/partial agonist has activity as a muscarinic M3 competitive antagonist. In some instances, the MAChR antagonist/inverse agonist/partial agonist has activity as a muscarinic M3 non-competitive antagonist. In some embodiments, a MAChR non-competitive antagonist ameliorates or otherwise reduces the undesired CNS effects of anti-muscarinics.

In some instances, the MAChR antagonist/inverse agonist/partial agonist has activity as a muscarinic M4 antagonist. In some instances, the MAChR antagonist/inverse agonist/partial agonist has activity as a muscarinic M43 inverse agonist. In particular instances, the MAChR antagonist/inverse agonist/partial agonist has activity as a muscarinic M4 partial agonist, for example, a partial agonist that displays antagonist activity in the presence of a full agonist (e.g., endogenous muscarinic receptor ligand). In particular instances, the MAChR antagonist/inverse agonist/partial agonist has activity as a muscarinic M4 competitive antagonist. In some instances, the MAChR antagonist/inverse agonist/partial agonist has activity as a muscarinic M4 non-competitive antagonist. In some embodiments, a MAChR non-competitive antagonist ameliorates or otherwise reduces the undesired CNS effects of anti-muscarinics.

In some instances, the MAChR antagonist/inverse agonist/partial agonist has activity as a muscarinic M5 antagonist. In some instances, the MAChR antagonist/inverse agonist/partial agonist has activity as a muscarinic M5 inverse agonist. In particular instances, the MAChR antagonist/inverse agonist/partial agonist has activity as a muscarinic M5 partial agonist, for example, a partial agonist that displays antagonist activity in the presence of a full agonist (e.g., endogenous muscarinic receptor ligand). In particular instances, the MAChR antagonist/inverse agonist/partial agonist has activity as a muscarinic M5 competitive antagonist. In some instances, the MAChR antagonist/inverse agonist/partial agonist has activity as a muscarinic M5 non-competitive antagonist. In some embodiments, a MAChR non-competitive antagonist ameliorates or otherwise reduces the undesired CNS effects of anti-muscarinics.

Certain embodiments employ “dual active” agents, or single agents that have dual activity as both a “MAChR antagonist/inverse agonist/partial agonist” and a “histamine H3 receptor antagonist/inverse agonist/partial agonist”, each of which is described separately herein. For instance, certain dual active agents agent have activity as a MAChR antagonist/inverse agonist/partial agonist:histamine H3 antagonist/inverse agonist/partial agonist at an IC50:IC50 ratio selected from about 1000:1, 900:1, 800:1, 700:1; 600:1, 500:1, 400:1, 300:1, 300:1, 200:1, 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:60, 1:70, 1:80, 1:90, 1:100, 1:200, 1:300, 1:400, 1:500, 1:600, 1:700, 1:800, 1:900, and 1:1000. The IC50 for each activity is measured in an vitro binding and/or functional assay as described herein.

In some embodiments, an agent of having dual activity as a MAChR antagonist/inverse agonist/partial agonist and a histamine H3 receptor antagonist/inverse agonist/partial agonist is selected from Table 1, including pharmaceutically-acceptable salts thereof. In some embodiments, the agent having dual activity as a MAChR antagonist/inverse agonist/partial agonist and a histamine H3 receptor antagonist/inverse agonist/partial agonist is not clemastine.

In particular embodiments, an agent having activity as a MAChR antagonist/inverse agonist/partial agonist (and in some instances, having little to no activity as a histamine H3 receptor antagonist/inverse agonist/partial agonist) is selected from Table 1. In certain of these and related embodiments, the agent having activity as a MAChR antagonist/inverse agonist/partial agonist (and in some instances, having little to no activity as a histamine H3 receptor antagonist/inverse agonist/partial agonist) are Atropine, Benztropine, Chlorpromazine, Clemastine, Dicyclomine, Diphenylpyraline, Disopyramide, Hyoscyamin, Mepenzolate, Orphenadrine, Oxybutynin, Paroxetine, Pentoxyverine, Piperidolate, Propafenone, Propiverine, Quetiapine, Scopolamine, Solifenacin, Tolterodine, Trihexyphenidyl, or Tripelennamine. In other embodiments, having activity as a MAChR antagonist/inverse agonist/partial agonist (and in some instances, having little to no activity as a histamine H3 receptor antagonist/inverse agonist/partial agonist) are benztropine, clemastine, oxybutynin, pentoxyverine, or propiverine.

Exemplary in vitro muscarinic receptor assays are known in the art. Examples of binding assays include radioligand binding assays such as [35S]-GTPγS binding assays, and examples of functional assays include cell-based calcium flux assays, for example, where antagonists decrease calcium (Ca2+) mobilization relative to controls (see, for example, Bymaster et al., Progress in Neuro-Psychopharm. & Biol. Psych. 27:1125-1143, 2003; and Dorje et al., Eur. J. Pharm. 203:417-420, 1991). Particular examples include cell-based functional assays using mammalian cells that support recombinant M1 expression on the cell surface for functional detection via the calcium signaling pathway, for example, using Calcium Flux FLIPR Assays (see, for example, Thomas et al., Neuropharmacology. 58:1206-14, 2010; and Zhang et al., Fitoterapia. 108:9-12, 2016). In specific embodiments, the IC50 as a muscarinic antagonist is measured in an in vitro functional calcium flux assay.

TABLE 1

| | CAS | | | | |
|---|---|---|---|---|---|
| | Registry | In vitro M1 | | OPC Cell | |
| Compound | No. | (Conc) | | Activity | Human Dosage |
| Atropine | 51-55-8 | 0.16 | nM | 5.7 uM | Oral 0.4 mg to 2 mg/day |
| Benztropine | 86-13-5 | 6.6 | nM | 170-350 nM | Oral 1 mg to 8 mg/day |
| Chlorpromazine | 50-53-3 | 22 | nM | Active | Oral 10 mg to 1 g/day |
| Clemastine | 15686-51-8 | 16 | nM | 140-780 nM | Oral 1 to 8 mg/day |
| Dicyclomine | 77-19-0 | 5 | nM | Active | Oral 20 mg to 120 mg/ |
| | | | | | day |
| Diphenylpyraline | 147-20-6 | 20 | nM | Active | Oral 2 mg to max 10 mg/ |
| | | | | | day |
| Disopyramide | 3737095 | | | | Oral up to 100 mg to 1 g/ |
| | | | | | day |
| Hyoscyamine | 101-31-5 | 8.2 | nM | Active | Oral 1.5 mg/day |
| Mepenzolate | 25990-43-6 | 27 | nM | Active | Oral 25 mg to 200 mg/ |
| | | | | | day |
| Orphenadrine | 83-98-7 | 57 | nM | Active | Oral 100 mg to 200 mg/ |
| | | | | | day |
| Oxybutynin | 5633-20-5 | 25 | nM | 0.67 uM | Oral 5 mg to 30 mg/day |
| Paroxetine | 61869-08-7 | 72 | nM | | Oral 10 mg to 50 mg/day |
| Pentoxyverine, | 77-23-6 | 13-76 | nM | 260 nM | Oral up to 180 mg/day |
| carbetapentane | | | | | |
| Piperidolate | 82-98-4 | 63 | nM | Active | Oral 100 to 200 mg/day |
| Propafenone | 54063-53-5 | | | Active | Oral 150 mg to 600 mg/ |
| | | | | | day |
| Propiverine | 60569-19-9 | 619 | nM | 420 nM | Oral 5 mg to 45 mg/day |
| Quetiapine | 111974- | 858 | nM | Active | Oral 100 mg to 800 mg/ |
| | 69-7 | | | | day |
| Scopolamine | 51-34-3 | 1 | nM | 5.1 uM | Patch 1 mg over 3 days |
| Solifenacin | 242478- | 25 | nM | Active | Oral 5 mg to 10 mg/day |
| | 37-1 | | | | |
| Tolterodine | 124937- | 190 | nM | | Oral 1 mg to 4 mg/day |
| | 51-5 | | | | |
| Trihexyphenidyl | 144-11-6 | 1.6 | nM | Active | Oral 1 mg to 15 mg/day |
| Tripelennamine | 91-81-6 | 1,3000 | nM | Active | Oral up to 600 mg/day |
| Acotiamide | 185106- | | | | |
| | 16-5 | | | | |
| Alimemazine | 84-96-8 | 398 | nM | | |
| Amatadine | 665-66-7 | >10 | uM | | Oral 100 to 200 mg/day |
| Amiodarone | 1951-25-3 | <10 | uM | | |
| Amitriptyline | 50-48-6 | 15 | nM | | Oral 10 mg to 300 mg/day |
| Aminobenzotropine | 47089-74-3 | | | Active | |
| Azelastine | 58581-89-8 | 4,200 | nM | | |
| Bevonium | 33371-53- | | | Active | Quat salt |
| | 8/5205- | | | | |
| | 82-3 | | | | |
| Biperiden | 514-65-8 | 0.85 | nM | | Oral 2 mg 3 to 16 mg/day |
| Caramiphen Edisylate | 77-22-5 | | | | Oral up to 800 mg/day |
| Chlorprothixene | 113-59-7 | 11 | nM | | Oral 5 mg to 100 mg/day |
| Citalopram | 59729-32-7 | 1430 | nM | | |
| Clozapine | 5786-21-0 | 12 | nM | | Oral 12.5 mg to 900 mg/ |
| | | | | | day |
| Compound I | 171805- | 27 | nM | | |
| | 25-7 | | | | |
| Cyamemazine | 3546-03-0 | 11 | nM | | Oral 50 mg to 300 mg/day |
| Difluoropine | 156774- | | | | |
| | 35-5 | | | | |
| Dosulepin | 113-53-1 | 18 | nM | | Oral 25 mg to 225 mg/day |
| Doxepin | 1668-19-5 | 0.12 | nM | | Oral 25 mg to 300 mg/day |
| Dronedarone (Multaq) | 141626- | | | | Oral 400 mg to 800 mg/ |
| | 36-0/ | | | | day |
| | 141625- | | | | |
| | 93-6 | | | | |
| Escitalopram | 128196- | 1240 | nM | Active | |
| | 01-0 | | | | |
| Glycopyrronium bromide | 596-51-0 | 0.42 | nM | | Quat salt |
| Hexahydroadiphenine | 1679-76-1 | | | | |
| Homatropine | 87-00-3 | | | | Eye drops 1-2 drops 2% |
| | | | | | solution, or 1 drop 5% |
| | | | | | solution, may repeat 10-15 min |
| | | | | | not to exceed 5 |
| | | | | | doses |
| Homochlorcyclizine | 848-53-3 | 22 | nM | | |
| Hydroxyzine | 68-88-2 | 3,800 | nM | Active | Risk of QT, Mixed OPC |
| | | | | | activity |
| Imipramine | 50-49-7 | 42 | nM | | Oral 25 mg to 300 mg/day |
| Ipratropium | 60205-81-4 | 1.31 | nM | Active | Quat salt |
| Isothipendyl | 482-15-5 | 74 | nM | | |
| Lofepramine | 23047-25-8 | 67 | nM | | Oral 70 mg to 210 mg/day |
| Lorcainide | 59729-31-6 | <10 | uM | | |
| Mebeverine | 630203 | | | | |
| Mebhydrolin | 524-81-2 | 180 | nM | | |
| Mepyramine | 91-84-9 | 30,000 | nM | | |
| Mequitazine | 29216-28-2 | 5.0 | nM | | |
| Methantheline | 5818-17-7 | | | | Quat salt |
| Methylatropine | 31610-87-4 | | | Active | Quat salt |
| Methylphenidate | 113-45-1 | | | | |
| Modafinil | 68693-11-8 | | | | |
| Muscarinic toxin 7 | 1379586- | | | | |
| | 13-6 | | | | |
| Octatropine methylbromide | 80-50-2 | | | Active | Quat salt Oral 50 mg to |
| | | | | | 300 mg/day |
| Octylonium bromide | 26095-59-0 | 2 | nM | Active | Quat salt Oral 20 mg to 80 mg/ |
| | | | | | day |
| Olanzapine | 132539- | 2.5-73 | nM | | Oral 5 mg to 20 mg/day |
| | 06-1 | | | | |
| Penehyclidine | 87827-02-9 | | | | |
| Profenamine | 522-00-9 | | | | |
| Promethazine | 60-87-7 | 26 | nM | | |
| Quinacrine | 69-05-6 | 460 | nM | | Oral 100 to 300 mg/day |
| Sertraline | 79617-96-2 | 430 | nM | | Oral 20 mg to 200 mg/day |
| SID 4258852 | | | | | |
| SID 4264322 | | | | | |
| Telenzepine | 80880-90-6 | | | | |
| Timepidium | 35035-05-3 | | | | Quat Salt |
| Tiotropium bromide | 136310- | 0.13 | nM | Active | Quat salt |
| | 93-5 | | | | |
| Triprolidine | 486-12-4 | 15000 | nM | | |
| Tropicamide | 1508-75-4 | | | | Eye drops 1-2 drops 2% |
| | | | | | solution, or 1 drop 5% |
| | | | | | solution, may repeat 10-15 min |
| | | | | | not to exceed 5 |
| | | | | | doses |
| VU0409774 | 1135243- | 690 | nM | | |
| | 19-4 | | | | |
| VU0409775 | 1416359- | | | | |
| | 27-7 | | | | |
| VU0414910 | not found | | | | |
| VU0415248 | 1392441- | | | | |
| | 79-0 | | | | |
| VU0431263 | 1413940- | | | | |
| | 38-1 | | | | |
| VU0433670 | 1413940- | 18 | nM | | |
| | 06-3 | | | | |
| VU0452865 | | | | | |
| VU0455691 | | | | | |
| VU6009229 | | 540 | nM | | |
| VU6009833 | | 288 | nM | | |
| Zamifenacin fumarate | 127308- | | | | |
| | 98-9 | | | | |
| Zotepine | 26615-21-4 | 18 | nM | | Oral 75 mg to 300 mg/day |
| Aclidinium bromide | 320345- | 0.10 | nM | | |
| | 99-1 | | | | |
| Astemizole | 68844-77-9 | | | | |
| AZD8683 | 1194737- | | | | |
| | 07-9 | | | | |
| AZD9164 | 1034978- | | | | |
| | 04-5 | | | | |
| Bencycloquidium bromide | 860804- | | | | |
| | 18-8/ | | | | |
| | 861000- | | | | |
| | 97-7 | | | | |
| Carbinoxamine | 486-16-8 | 740 | nM | | |
| CHF 5407 | 1159397- | | | | |
| | 54-2 | | | | |
| Chlorpheniramine | 132-22-9 | 2.6 | nM | | |
| Cibenzoline | 53267-01-9 | | | | |
| Cyproheptadine | 129-03-3 | 8.8 | nM | | |
| Dicycloverine | 77-19-0 | | | | |
| Dimenhydrinate | 523-87-5 | 160 | nM | | |
| Dimetindene | 5636-83-9 | 320 | nM | | |
| Doxylamine | 469-21-6 | | | | |
| Femoxetine | 59859-58-4 | | | | |
| Fluperlapine | 67121-76-0 | | | | |
| Haloperidol | 52-86-8 | >2000 | nM | | |
| Ketotifen | 34580-13-7 | 260 | nM | | |
| Mesoridazine | 5588-33-0 | 10 | nM | | |
| Methapyrilene | 91-80-5 | 2.8 | nM | | |
| Nortriptyline | 72-69-5 | 40 | nM | | |
| Pirmenol | 68252-19-7 | | | | |
| Revatropate | 149926- | | | | |
| | 91-0 | | | | |
| TD-4208 | 864750- | | | | |
| | 70-9 | | | | |
| Terfenadine | 50679-08-8 | | | | |
| Thioridazine | 50-52-2 | | | | |
| V0162 | 869113- | | | | |
| | 09-7 | | | | |
| Vamicamide | too many | | | | |
| (+)-Himbacine | 6879-74-9 | | | | |
| 4-Diphenylacetoxy-N- | 1952-15-4 | | | | |
| methylpiperidinemethiodide | | | | | |
| (4-DAMP) | | | | | |
| AF-DX 116 | 102394- | 417 | nM | | |
| | 31-0 | | | | |
| AF-DX 384 | 118290- | | | | |
| | 26-9 | | | | |
| AWD 26-06 | 98374-54-0 | | | | |
| Cyclobenzaprine | 303-53-7 | | | | |
| Darifenacin | 133099- | | | Active | |
| | 04-4 | | | | |
| Diphemanil | 62-97-5 | | | | |
| Diphenhydramine | 34402-86-3 | 280 | nM | | |
| Fenpiverinium | 258329- | | | | |
| | 46-3 | | | | |
| Fesoterodine | 286930- | | | | |
| | 02-7 | | | | |
| Flavoxate | 15301-69-6 | | | | |
| Gallamine | 65-29-2 | | | | |
| Hexahydro-sila-difenidol | 98299-40-2 | | | | |
| Hexocyclium | 6004-98-4/ | 1.2 | nM | | |
| | 115-63-9 | | | | |
| Imidafenacin | 170105- | | | | |
| | 16-5 | | | | |
| Methoctramine | 104807- | | | | |
| | 46-7 | | | | |
| Oxyphenonium | 14214-84-7 | | | | |
| PCS1055 | 357173- | | | | |
| | 55-8 | | | | |
| p-Fluorohexahydro-sila- | 116679- | 17 | nM | | |
| difenidol | 83-5 | | | | |
| Pipenzolate | 13473-38-6 | | | | |
| Pirenzepine | 28797-61-7 | | | | |
| Procyclidine | 77-37-2 | | | | |
| Propantheline | 298-50-0 | 0.22 | nM | 1.25-2.95 uM | |
| Propiomazine | 362-29-8 | | | | |
| Temiverine | 173324- | | | | |
| | 94-2 | | | | |
| Tripitramine | 152429- | | | | |
| | 64-6 | | | | |
| Trospium chloride | 1508-75-4 | | | | |
| Umeclidinium bromide | 10405-02-4 | | | | |
| VU0255035 (ML012) | 132373- | | | | |
| | 81-0 | | | | |
| YM-46303 | 171723- | | | | |
| | 79-8 | | | | |

### Histamine H3 Receptor Antagonists/Inverse Agonists/Partial Agonists

Histamine receptors are proteins situated in various parts of the body that bind with histamine to produce a specific effect on the organism. There are four known receptors, designated H1, H2, H3 and H4. The receptor that the histamine reacts with is dependent upon where the histamine is released in the body.

H1 receptors are one of the most important receptors for modulating your internal clock, and are a main target for many clinical drugs. When histamine reacts with these receptors in your brain it alters your neurochemistry to make you more awake and alert.

H2 receptors are found on parietal cells located in the stomach lining, and are mainly responsible for regulating the levels of gastric acid. Histamine action at these receptors stimulates the release of gastric acid, excess of which can result in gastroenteritis. These receptors are also found on heart, uterus and vascular smooth muscle cells.

H4 receptors regulate the levels of white blood cell release from bone marrow. They have also been shown to direct mast cells. They are located in the thymus, small intestine, spleen, the colon, bone marrow and basophils. The H4 receptor shares about 40% homology with H3 receptor.

H3 receptors are present throughout the nervous system, though most notably in the central nervous system. They regulate histamine in the body, by inhibiting the further synthesis of histamine. The more of these receptors that are triggered by histamine, the less histamine is produced in the body.

Specifically, the H3 receptor (H3R; 326-445 amino-acids) is located on histaminergic neuron somata, dendrites and axon varicosities, as well as on the axon varicosities and somata of other neurons; it is coupled to Gi/o to inhibit adenylyl cyclase and the high voltage activated Ca2+ channels. The histamine H4 receptor is predominantly expressed in immune cells, including mast cells, eosinophils, and dendritic cells.

Histamine H3 receptors are expressed in the central nervous system and to a lesser extent the peripheral nervous system, where they act as autoreceptors in presynaptic histaminergic neurons, and also control histamine turnover by feedback inhibition of histamine synthesis and release. The H3 receptor also presynaptically inhibits the release of neurotransmitters including dopamine, GABA, acetylcholine, noradrenaline, histamine, and serotonin. The histamine H3 receptor is a G-protein coupled receptor, which is coupled to the Gi G-protein and thereby inhibits the formation of cAMP. The β and γ subunits of the histamine H3 receptor interact with N-type voltage gated calcium channels to reduce action potential-mediated influx of calcium and thereby reduce neurotransmitter release. H3 receptors function as presynaptic autoreceptors on histamine-containing neurons.

Thus, in some instances, a histamine H3 receptor antagonist/inverse agonist/partial agonist (also referred to herein as a H3 antagonist/inverse agonist/partial agonist) reduces or inhibits the activity of histamine H3 receptors in a cell, tissue, or subject, for example, by about or at least about 5, 10, 20, 30, 40, 50, 60, 70, 80, 90, or 100%, relative to a control, for example relative to a baseline level of activity.

In particular embodiments, an agent having activity as a histamine H3 receptor antagonist/inverse agonist/partial agonist has an IC50 in an in vitro H3 receptor-based binding and/or functional assay ranging from about 0.10-100000 nM, 0.10-10000 nM, 0.1-1000 nM, 0.1-500 nM, 0.1-400 nM, 0.1-300 nM, 0.1-200 nM, 0.1-100 nM, 0.1-90 nM, 0.1-80 nM, 0.1-70 nM, 0.1-60 nM, 0.1-50 nM, 0.1-40 nM, 0.1-30 nM, 0.1-20 nM, 0.1-10 nM, 0.1-5 nM, 0.1-1.0 nM, or 0.1-0.5 nM, or about or no more than about 100000 nM, 10000 nM, 1000 nM, 500 nM, 400 nM, 300 nM, 200 nM, 100 nM, 90 nM, 80 nM, 70 nM, 60 nM, 50 nM, 40 nM, 30 nM, 20 nM, 10 nM, 5 nM, 1 nM, 0.9 nM, 0.8 nM, 0.7 nM, 0.6 nM, 0.5 nM, 0.4 nM, 0.3 nM, 0.2 nM, 0.1 nM, or 0.05 nM.

In some instances, a histamine H3 antagonist/inverse agonist/partial agonist has selective activity towards the histamine H3 receptor relative to histamine H1, H2, and/or H4 receptors. Without being bound by any one theory, in some instances, selective activity towards the histamine H3 receptor improves the undesirable side effects cause by H1, H2, H4 inhibition, including, for example, sedation (H1), GI Acid reduction (H2), and Chemotaxis (H4).

In some instances, the histamine H3 antagonist/inverse agonist/partial agonist has activity as a histamine H3 receptor antagonist. In some instances, the histamine H3 antagonist/inverse agonist/partial agonist has activity as a histamine H3 inverse agonist. In particular instances, the histamine H3 antagonist/inverse agonist/partial agonist has activity as a histamine H3 partial agonist, for example, a partial agonist that displays antagonist activity in the presence of a full agonist (e.g., endogenous histamine H3 receptor ligand). In particular instances, the histamine H3 antagonist/inverse agonist/partial agonist has activity as a histamine H3 competitive antagonist. In some instances, the histamine H3 antagonist/inverse agonist/partial agonist has activity as a histamine H3 non-competitive antagonist.

Exemplary agents having activity as a histamine H3 receptor antagonist/inverse agonist/partial agonist are provided in Table 2 below, including pharmaceutically-acceptable salts thereof.

In some embodiments, an agent of having as a histamine H3 receptor antagonist/inverse agonist/partial agonist is ABT-288, Bavisant, GSK239512, Irdabisant, MK-0249 or pitolisant.

In some instances, an agent having activity as a histamine H3 antagonist/inverse agonist/partial agonist has selective activity towards one or more histamine H3 isoforms relative to one or more other histamine H3 isoforms (see, for example, Hancock et al., Life Sci. 73:3043-72, 2003). For example, certain agents have selective activity towards a long isoform of the histamine H3 receptor relative to other isoforms, for example, relative to one or more short isoforms. Some agents have selective activity towards one or more short isoforms of the histamine H3 receptor relative to other histamine H3 receptor isoforms, for example, relative to one or more long isoforms. In certain embodiments, the one or more short isoforms of the histamine H3 receptor comprise one or more deletions in the third intracellular loop.

Exemplary assays for measuring the activity of histamine H3 receptor antagonists/inverse agonists are known in the art. Examples of binding assays include in vitro radioligand binding assays such as [35S]-GTPγS binding assays and others (see, for example, Singh et al., Ann Neurosci. 19:71-5, 2012; Lim et al., J. of Pharm. & Exp. Therap. 314:1310-1321, 2005; and Laitinen and Jokinen, J. of Neurochem. 71:808-816, 1998), and in vivo assays using radiotracers to provide measurements of H3 receptor occupancy (see, for example, Miller et al., Br J Pharmacol. 157:139-49, 2009). Examples of in vitro functional assays include colorimetric cyclic AMP (cAMP) assays, for example, where antagonists or inverse agonists reduce cAMP modulation relative to controls (see, for example, Lim et al., J. of Pharm. & Exp. Therap. 314:1310-1321, 2005). In specific embodiments, the IC50 as a histamine H3 antagonist/inverse agonist is measured in an in vitro functional cAMP assay.

TABLE 2

| | | | Partial agonist | | |
|---|---|---|---|---|---|
| | | | vs Antagonist | | |
| | | pKi | vs Inverse | OPC | |
| | | or | agonist/Protean | Cell | Human |
| Compound | CAS | IC50 | Agonist | Activity | Dose |
| A-317920 | 360551- | 7 | Inverse agonist | | |
| | 59-3 | | | | |
| A-320436 | 378233- | 8.8 | Antagonist | | |
| | 39-7 | | | | |
| A-331440 | 392338- | 8.56 | Inverse agonist | | n/a |
| | 13-5 | | | | |
| ABT-249 | 460746- | 9.35 | Inverse agonist | | n/a |
| | 46-7 | | | | |
| ABT-288 | 948845- | 8.7 | Antagonist | | Oral, 1-25 mg/ |
| | 91-8 | | | | day |
| ABT-834 | | | | | |
| AZD-5213 | 1476077- | 9.3 | Inverse agonist | | Oral 0.5 mg/ |
| | 02-7 | | | | day |
| Betahistine | 5638-76-6 | 0.1 nM | Inverse agonist | | Oral 8-48 mg/ |
| | | | | | day |
| CEP-26401 | 1005402- | 9.3 | Inverse agonist | 250 nM | Oral |
| (Irdabisant) | 19-6 | | | | 0.02-5 mg/ |
| | | | | | day |
| CEP-32215 | 1174934- | 2.0 nM | Inverse agonist | | n/a |
| | 45-2 | | | | |
| Ciproxifan | 184025- | 7.1 | Inverse agonist | | n/a |
| | 18-1 | | | | |
| Contilisant | 2135615- | 10.8 | | | |
| | 68-6 | | | | |
| FUB-138 | 152029- | 7.9 | Antagonist | | n/a |
| | 32-8 | | | | |
| FUB-153 | 152029- | 7.8 | Antagonist | | n/a |
| | 04-4 | | | | |
| FUB-181 | 152029- | 8.1 | Antagonist | | n/a |
| | 80-6 | | | | |
| FUB-833 | 409127- | 9.5 | Antagonist | | n/a |
| | 71-5 | | | | |
| GSK- | 955359- | 10.6 | Antagonist | | Liquid |
| 1004723 | 72-5 | | | | 200-1000 mg/ |
| | | | | | day |
| GSK-189254 | 720690- | 9.9 | Inverse agonist | | Oral 50-100 |
| | 73-3 | | | | ug/day |
| GSK-239512 | 720691- | 10 | Antagonist | 120-160 nM | 10-80 |
| | 69-0 | | | | ug/day |
| GSK-247246 | 1448422- | ? | Inverse agonist | 25-100 nM | n/a |
| | 61-4 | | | | |
| GSK-334429 | 799557- | 9.5 | Antagonist | | |
| | 57-6 | | | | |
| GSK-835726 | | 7.8 | Antagonist | | Oral 10-100 mg/ |
| | | | | | day |
| GT2331 | 223420- | | | | |
| | 11-9 | | | | |
| JNJ- | 929622- | 8.4 | Antagonist | | Oral 0.5-30 mg/ |
| 31001074/ | 08-2 | | | | day |
| Bavisant | | | | | |
| JNJ- | 959740- | 8.8 | Antagonist | | Oral |
| 39220675 | 39-7 | | | | solution |
| | | | | | 10 mg/ |
| | | | | | day |
| JNJ-5207852 | 398473- | 9.24 | Inverse agonist | | |
| | 34-2 | | | | |
| JNJ-6379490 | 365565- | 2.0 nM | | | |
| | 36-2 | | | | |
| MK-0249 | 1167574- | 8.2 | Inverse agonist | | Oral 3- |
| | 41-5 | | | | 12 mg/ |
| | | | | | day |
| MK-3134 | 862310- | 3.6 nM | Inverse agonist | | Oral 25 mg/ |
| | 66-5 | | | | day |
| MK-7288 | 936626- | | Inverse agonist | | Oral 10-20 mg/ |
| | 07-2 | | | | day |
| NNC 38- | 757183- | 2.3 nM | Antagonist | | |
| 1049 | 18-9 | | | | |
| PF-03654746 | 935840- | 8.6 | Antagonist | | Oral |
| | 31-6 | | | | 0.25-2 mg/ |
| | | | | | day |
| PF-03654764 | 935840- | 8.84 | Antagonist | | Oral 5 mg/ |
| | 35-0 | | | | day |
| Pitolisant | 362665- | 8.06 | Inverse agonist | 14-100 nM | Oral 9-36 mg/ |
| (tiprolisant) | 56-3 | | | | day |
| S 38093 | 862896- | 1.2 μM | Inverse agonist | | |
| | 30-8 | | | | |
| SAR110068 | | 1 nM | Inverse agonist | | |
| SAR-110894 | 1167574- | 0.48 nM | Inverse agonist | | Oral 5-10 mg/ |
| | 45-9 | | | | day |
| SUVN | 1394808- | | | | |
| G3031 | 82-2 | | | | |
| UCL 1390 | 152030- | 12 nM | Antagonist | | |
| | 48-3 | | | | |
| UW-MD-71 | 1539279- | 76 nM | Inverse agonist | | |
| | 45-2 | | | | |
| | 1035626- | | | | Oral 50 mg/ |
| | 05-1 | | | | day |
| ADP916 | 1021169- | 0.7 nM | Inverse agonist | | |
| | 11-8 | | | | |
| ADS-003 | 1589534- | 8.2 | Inverse agonists | | |
| | 01-9 | | | | |
| Compound | 1146699- | | Inverse agonist | | |
| 2q | 85-5 | | | | |
| Compound | 2133824- | 7.6 nM | | | |
| 20 | 69-6 | | | | |
| Compound | 866265- | 0.5 nM | | | |
| 46 | 43-2 | | | | |
| Compound | 939030- | 11 nM | | | |
| 47 | 08-7 | | | | |
| Conessine | 546-06-5 | 8.27 | Inverse agonist | | |
| DL-77 | | 5.3 nM | | | |
| E177 | | | | | |
| SCH-79687 | 224585- | | | | |
| | 45-9 | | | | |
| ST-1283 | 1186310- | | | | |
| | 77-9 | | | | |
| UW-MD-72 | 1539279- | 2.54 μM | | | |
| | 30-5 | | | | |
| Amiodarone | 1951-25-3 | 7.5 | Inverse agonist | | Oral |
| | | | | | 200-1600 mg/ |
| | | | | | day |
| Aplysamine | 159026- | 0.83 uM | | | |
| | 30-9 | | | | |
| Clobenpropit | 145231- | 9 | Inverse agonist | 125 nM | |
| | 45-4 | | | | |
| Compound | 40964- | | | | |
| 10 | 08-7 | | | | |
| Compound | 1093179- | | | | |
| 12 | 73-7 | | | | |
| Compound | 73903- | 5.4 | | | |
| 12 | 17-0 | | | | |
| Dimaprit | 65119- | | | | |
| | 89-3 | | | | |
| GT-2331 | 223420- | 5.2 nM | Protean agonist | | |
| (Perceptin) | 11-9 | | | | |
| (Cipralisant) | | | | | |
| Imetit | 102203- | | Mostly agonist | | |
| | 18-9 | | | | |
| Iodoproxyfan | 152028- | 5 nM | Protean agonist | | |
| | 96-1 | | | | |
| Lorcainide | 59729- | 7 | Inverse agonist | | IV 200-300 mg/ |
| | 31-6 | | | | day |
| | | | | | (P2); |
| | | | | | Oral 300 mg/ |
| | | | | | day |
| | | | | | (P1) |
| OUP-186 | 1480830- | 8.1 | Antagonist | | |
| | 24-7 | | | | |
| Proxyfan | 177708- | | Protean agonist | | |
| | 09-7 | | | | |
| S 387618 | | 8.1 | | | |
| Thioperamide | 106243- | 7.6 | Inverse agonist | | |
| | 16-7 | | | | |
| UCL 1972 | 220728- | 39 | | | |
| | 17-6 | | | | |
| Clemizole | 442-52-4 | | | Active | |
| Doxylamine | | | | | |
| GSK-20 | | | | | |
| JNJ- | 1046447- | | | | |
| 39758979 | 90-8 | | | | |
| JNJ-7777120 | 459168- | | | | |
| | 41-3 | | | | |
| ZPL- | 943057- | | | | |
| 3893787 | 12-3 | | | | |

### Methods of Use

In certain embodiments, the present disclosure relates to inducing, promoting, or enhancing the differentiation and/or proliferation of Oligodendrocyte Progenitor Cells (OPCs) into mature oligodendrocytes to create new myelin sheaths on demyelinated axons in the central nervous system (CNS) and peripheral nervous system (PNS).

When OPCs are treated with an agent in accordance to the methods of the invention, whether the population is in vivo or in vitro, the treated OPCs have the capacity to proliferate and/or differentiate and, more specifically, differentiate into oligodendrocytes. In some instances, an agent induces and maintains the OPCs to produce daughter OPCs that can divide for many generations and maintain the ability to have a high proportion of the resulting cells differentiate into oligodendrocytes. In certain embodiments, the proliferating OPCs express progenitor cell marker(s) selected from one or more of NG2, PDGFR-alpha, A2B5, NKx2.2

In some embodiments, the methods may be used to maintain, or even transiently increase self-renewal of a pre-existing progenitor cell population prior to significant myelin sheath formation.

Morphological analyses with immunostaining (including cell counts) and lineage tracing across a Representative Microscopy Samples may be used to confirm expansion of the OPCs. Morphological analyses with immunostaining (including cell counts) and qPCR and RNA hybridization may be used to confirm upregulation of markers of mature oligodendrocytes, including CC1, MOG, MBP, PLP, CNPase amongst the cell population.

Advantageously, methods described herein can achieve these goals without the use of genetic manipulation. Germ-line manipulation used in many academic studies is not a therapeutically desirable approach to treating demyelination. In general, the therapy preferably involves the administration of a small molecule, peptide, antibody, or other non-nucleic acid molecule or nucleic acid delivery vector unaccompanied by gene therapy. In certain embodiments, the therapy involves the administration of a small organic molecule. In some instances, remyelination is achieved through the use of a (non-genetic) therapeutic that delivered for example orally.

Thus, in various aspects the invention provides method of increasing OPCs proliferation and/or differentiation; producing an expanded population of oligodendrocyte; increasing remyelination and treating demyelination disorder in a subject by contacting a OPCs or administering to the subject MAChR antagonist/inverse agonist/partial agonist together with a H3 antagonist/inverse agonist/partial agonist.

In some embodiments OPC differentiation is induced and or increased by contacting OPCs, with an agent(s) having MAChR antagonist/inverse agonist/partial agonist activity and a H3 antagonist/inverse agonist/partial agonist activity.

Also included are methods of inducing and/or increasing OPC differentiation by contacting neural tissue neural tissue containing OPCs, with an agent(s) having MAChR antagonist/inverse agonist/partial agonist activity and a H3 antagonist/inverse agonist/partial agonist activity.

In some embodiments OPC cell population is expanded by contacting OPCs, with an agent(s) having MAChR antagonist/inverse agonist/partial agonist activity and a H3 antagonist/inverse agonist/partial agonist activity.

Also included are methods for expanding a population of oligodendrocytes in a neural tissue neural tissue by contacting the neural tissue neural tissue containing OPCs with an agent(s) having MAChR antagonist/inverse agonist/partial agonist activity and a H3 antagonist/inverse agonist/partial agonist activity.

In some embodiments remyelination of a neuronal axon is increased by contacting the neuronal axon, with an agent(s) having MAChR antagonist/inverse agonist/partial agonist activity and a H3 antagonist/inverse agonist/partial agonist activity.

Also included are methods of inducing and/or increasing remyelination of a neuronal axon by contacting neural tissue axon with an agent(s) having MAChR antagonist/inverse agonist/partial agonist activity and a H3 antagonist/inverse agonist/partial agonist activity.

In the various methods OPC differentiation or expansion is increases compared to a vehicle control.

In some embodiments, the an agent(s) having MAChR antagonist/inverse agonist/partial agonist activity and a H3 antagonist/inverse agonist/partial agonist activity increases OPC differentiation by at least 10, 20, 30, 40, 50, 60, 70, 80, 90, 100, 150, 200, 250, 300, 350, 400, 450, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more), relative to a vehicle control.

In some embodiments, the an agent(s) having MAChR antagonist/inverse agonist/partial agonist activity and a H3 antagonist/inverse agonist/partial agonist activity increases OPC proliferation and therefor expanded a population of oligodendrocytes by at least 10, 20, 30, 40, 50, 60, 70, 80, 90, 100, 150, 200, 250, 300, 350, 400, 450, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more), relative to a vehicle control.

Remyelination is the process of propagating, proliferating, differentiating, and/or migration of oligodendrocyte precursor cells to form oligodendrocytes and thereby create new myelin sheaths on demyelinated axons in the peripheral or central nervous system. Typically, evidence that remyelination has taken place on an axon includes the creation of a relatively thin myelin sheath by oligodendrocyte(s), which can be quantified by the “g-ratio”, that is, the ratio between the diameter of the axon itself relative to the outer diameter of the myelinated fiber. Alternatively, that evidence of remyelination has taken place on an axon included determining the percentage of axons that are myelinated compared to control.

Exemplary models for evaluating myelination, demyelination, and remyelination are described, for example, in Osorio-Querejeta et al. (Neuromolecular Med. 19:181-192, 2017). Exemplary methods for evaluating remyelination in vivo include the use of magnetization transfer ratio (MTR) and myelin water fraction and diffusion tensor imaging (DTI) metrics, as described, for example, in Mallik et al., (J Neurol Neurosurg Psychiatry. 85:1396-404, 2014). Another approach, is to look at the latency of visually evoked potentials.

Thus, in some embodiments, the methods of the invention increases remyelination in the subject in need thereof by about or at least about 5, 10, 20, 30, 40, 50, 60, 70, 80, 90, 100, 110%, 120%, 130%, 140%, 150%, 160%. 170%, 180%, 190% 200%, 300%, 400%, 500% or more relative to a control (for example, a reference standard or earlier time point).

In other embodiments, the methods of the invention increases remyelination in the subject in need thereof to alleviate one or more signs or symptom of a demyelinating disorders. In some embodiments demyelinating disorders are treated by administering the subject an agent(s) having MAChR antagonist/inverse agonist/partial agonist activity and a H3 antagonist/inverse agonist/partial agonist activity.

Also included are methods treating demyelinating in a subject by contacting the subjects neural tissue with an agent(s) having MAChR antagonist/inverse agonist/partial agonist activity. In some embodiments, the agent having MAChR antagonist/inverse agonist/partial agonist activity and H3 antagonist/inverse agonist/partial agonist activity are the same agent. Put another way, the agent has “dual activity.

By a “dual activity” agent is meant that the agent has both MAChR antagonist/inverse agonist/partial agonist activity and H3 antagonist/inverse agonist/partial agonist activity. In some embodiments, the dual activity agent is selected from Table 1. In some embodiments. the dual activity agent is not clemastine.

In other embodiments, the agent having MAChR antagonist/inverse agonist/partial agonist activity and H3 antagonist/inverse agonist/partial agonist activity are different agents. In some embodiments, an agent having MAChR antagonist/inverse agonist/partial agonist activity is selected from Table 1. For example, the agent is Atropine, Benztropine, Chlorpromazine, Clemastine, Dicyclomine, Diphenylpyraline, Disopyramide, Hyoscyamin, Mepenzolate, Orphenadrine, Oxybutynin, Paroxetine, Pentoxyverine, Piperidolate, Propafenone, Propiverine, Quetiapine, Scopolamine, Solifenacin, Tolterodine, Trihexyphenidyl, or Tripelennamine. In other embodiments, having activity as a MAChR antagonist/inverse agonist/partial agonist (and in some instances, having little to no activity as a histamine H3 receptor antagonist/inverse agonist/partial agonist) is benztropine, clemastine, oxybutynin, pentoxyverine, or propiverine.

In some embodiments, agent having H3 antagonist/inverse agonist/partial agonist activity is selected from Table 2. For example, the agent is ABT-288, Bavisant, GSK239512, Irdabisant, MK-0249 or pitolisant.

In certain embodiments the agent having MAChR antagonist/inverse agonist/partial agonist activity and the agent H3 antagonist/inverse agonist/partial agonist activity are administered concurrently. For example, the agent having MAChR antagonist/inverse agonist/partial agonist activity and the agent H3 antagonist/inverse agonist/partial agonist activity are administered in the same pharmaceutical composition, optionally as described herein. Alternatively the agent having MAChR antagonist/inverse agonist/partial agonist activity and the agent H3 antagonist/inverse agonist/partial agonist activity are administered separately For example, the agent having MAChR antagonist/inverse agonist/partial agonist activity and the agent H3 antagonist/inverse agonist/partial agonist activity are administered in separate pharmaceutical compositions. In some embodiments, the subject has, or is at risk for having, a demyelinating disease or condition. A demyelinating disease is any disease of the nervous system in which the myelin sheath of neurons is damaged. This damage impairs the conduction of signals in the affected nerves. In turn, the reduction in conduction ability causes deficiency in sensation, movement, cognition, or other functions depending on which nerves are involved. In particular embodiments, the subject has, or is at risk for having, a demyelinating disease of the central nervous system (CNS). In some embodiments, the subject has, or is at risk for having, a demyelinating disease of the peripheral nervous system. In certain embodiments, the subject has a disease or condition that would benefit from increased remyelination.

Subjects having, or at risk for having, a demyelinating disease or a related disease of interest can be identified or diagnosed according to routine techniques in the art. For example, magnetic resonance imaging (MM) can be used to visualize internal structures of the body in detail, and assess changes in proton density; “spots” can occur as a result of changes in brain water content (see, e.g., Freedman, Mark S. (2005). Advances in Neurology Volume 98: Multiple Sclerosis and Demyelinating Diseases. Philadelphia: Lippincott Williams & Wilkins). Clinicians can employ evoked potential techniques, using an electrical potential recorded from the nervous system following the presentation of a stimulus as detected by electroencephalography (EEG), electromyography (EMG), or other electrophysiological recording method (see Freedman, 2005, supra). Cerebrospinal fluid analysis (CSF) can be useful in the diagnosis of central nervous system infections. A CSF culture examination may yield the microorganism that caused the infection. Quantitative proton magnetic resonance spectroscopy (MRS) is a non-invasive analytical technique that has been used to study metabolic changes in brain tumors, strokes, seizure disorders, Alzheimer's disease, depression and other diseases affecting the brain. Diagnostic criteria refers to a specific combination of signs, symptoms, and test results that the clinician can use to determine the correct diagnosis (see Freedman, 2005, supra). Also, fluid-attenuated inversion recovery (FLAIR) uses a pulse sequence to suppress cerebrospinal fluid and show lesions more clearly, and can be used, for example, in multiple sclerosis evaluations.

In specific embodiments, the disease or condition to be treated in accordance to the method of the disclosure is Acute disseminated encephalomyelitis (ADEM); Acute hemorrhagic leukoencephalitis; Acute optic neuritis; Acute transverse myelitis; Adrenoleukodystrophy; Adrenomyeloneuropathy; Alexander Disease; Alzheimer's Disease; aminoacidurias; Amyotrophic Lateral Sclerosis; Anti-MAG peripheral neuropathy; Anti-MOG associated spectrum; Balo concentric sclerosis; Brain injury; CAMFAK Syndrome; Canavan Disease; Carbon monoxide toxicity; Central pontine myelinolysis; Cerebral hypoxia; Cerebral ischemia; Charcot-Marie-Tooth disease; Chronic inflammatory demyelinating polyneuropathy; Chronic relapsing inflammatory optic neuritis (CRION); Chronic traumatic encephalopathy; clinically isolated syndrome (CIS); Congenital Cataract; Copper deficiency associated condition; Delayed Post-Hypoxic Leukoencephalopathy; diffuse cerebral sclerosis of Schilder; diffuse myelinoclastic sclerosis; extrapontine myelinolysis; Gaucher disease; Guillain-Barré syndrome; Hereditary neuropathy; hereditary neuropathy with liability to pressure palsy; HTLV-1-associated myelopathy; Hurler syndrome; Hypomyelination; hypoxic brain injury; Krabbe Disease; Leber hereditary optic atrophy and related mitochondrial disorders; leukodystrophic disorders; Marburg multiple sclerosis; Marchiafava-Bignami disease; Metachromatic leukodystrophy; multiple sclerosis; multiple system atrophy; myelinoclastic disorders; myelopathy; nerve injury; neuromyelitis optica; Neuromyelitis optica (NMO); Niemann-Pick disease; optic neuropathy; optic-spinal multiple sclerosis; Osmotic Demyelination Syndrome; Parkinsons; Pelizaeus-Merzbacher Disease; peripheral neuropathy; Phenylketonuria; primary progressive multiple sclerosis (PPMS); progressive inflammatory neuropathy; progressive multifocal leukoencephalopathy; Progressive subcortical ischemic demyelination; progressive-onset multiple sclerosis; relapsing-onset multiple sclerosis; relapsing-remitting multiple sclerosis (RRMS); reperfusion injury; Schilder disease; secondary progressive multiple sclerosis (SPMS); Solitary sclerosis; Spinal Cord Injury; Subacute sclerosing panencephalitis; Tabes dorsalis; Tay-Sachs disease; Traumatic Brain Injury; Tropical spastic paraparesis; Tumefactive multiple sclerosis; or Vitamin B12 deficiency.

Exemplary signs and symptoms of, a demyelinating disease or condition include, without limitation, blurred double vision (Diplopia), ataxia, clonus, dysarthria, fatigue, clumsiness, hand paralysis, hemiparesis, genital anaesthesia, cognitive impairment, incoordination, paresthesias, ocular paralysis (cranial nerve palsy), impaired muscle coordination, weakness (muscle), loss of sensation, impaired vision, neurological symptoms, unsteady gait, spastic paraparesis, incontinence, hearing loss, and speech dysfunction. In some embodiments, the methods of the invention improves one or more of the foregoing symptoms in the subject, among others.

In particular embodiments, the activity as the histamine H3 antagonist/inverse agonist/partial agonist reduces one or more side effects of the activity of the MAChR antagonist/inverse agonist/partial agonist. In some embodiments, the one or more side effects are selected from drowsiness, dry mouth, visual impairment or distortion, constipation, central nervous system (CNS) impairment, and cognitive impairment.

In some embodiments, adding an H3 antagonist to a muscarinic antagonist allows for a comparable activation of OPCs to be achieved in the OPC Differentiation Assay even at a reduced concentration of the MAChR antagonist/inverse agonist/partial agonist. In some embodiments, the concentration of the MAChR antagonist/inverse agonist/partial agonist. may be reduced by 50, 100, 200, or 500% when used in combination with an H3 antagonist/inverse agonist/partial agonist while maintaining comparable efficacy to the MAChR antagonist/inverse agonist/partial agonist alone in the OPC differentiation assay.

In various embodiments the agent(s) having MAChR antagonist/inverse agonist/partial agonist activity and a H3 antagonist/inverse agonist/partial agonist activity are administered to the subject systemically or locally. Systemic administration includes, but is not limited, to oral or parenteral administration. Parenteral routes include for example intramuscular (IM), subcutaneous (SC), dermal, and intravenous (IV). Local administration includes for example, intracerebroventricular (ICV), intrathecal, intraocular, or via eye drops. More specific methods of delivery are described herein.

In some embodiments, both the MAChR antagonist/inverse agonist/partial agonist and the H3 antagonist/inverse agonist/partial agonist are administered locally. In other embodiments, both the MAChR antagonist/inverse agonist/partial agonist and the H3 antagonist/inverse agonist/partial agonist are administered systemically. In some embodiments the MAChR antagonist/inverse agonist/partial agonist is administered locally and the H3 antagonist/inverse agonist/partial agonist is administered systemically. In other embodiments the MAChR antagonist/inverse agonist/partial agonist is administered systemically and the H3 antagonist/inverse agonist/partial agonist is administered locally.

In some embodiments, the MAChR antagonist/inverse agonist/partial agonist and a H3 antagonist/inverse agonist/partial agonist are administered at the same time. In other embodiments, the MAChR antagonist/inverse agonist/partial agonist and a H3 antagonist/inverse agonist/partial agonist are administered at different times. In some embodiments the MAChR antagonist/inverse agonist/partial agonist is administered a a period of time before the a H3 antagonist/inverse agonist/partial agonist. In other embodiments, the MAChR antagonist/inverse agonist/partial agonist is administered at a period of time after the H3 antagonist/inverse agonist/partial agonist. For example, the MAChR antagonist/inverse agonist/partial agonist is administered 1, 2, 3, 4, 5, 6, 7, 8, 9, 10, 12, 13, 14, 14, 15, 17, 18, 19, 20, 21, 22, 23, 24 hours or 1, 2, 3, 4, 5, 6, 7 or more days before the H3 antagonist/inverse agonist/partial agonist. Alternatively, the MAChR antagonist/inverse agonist/partial agonist is administered 1, 2, 3, 4, 5, 6, 7, 8, 9, 10, 12, 13, 14, 14, 15, 17, 18, 19, 20, 21, 22, 23 or 24 hours or 1, 2, 3, 4, 5, 6, 7 or more days before the H3 antagonist/inverse agonist/partial agonist or after the H3 antagonist/inverse agonist/partial agonist.

Demyelinating disease or disorders or reduced neuronal function is treated or prevented utilizing the various methods described herein to increase OPC proliferation and/or differentiation. The OPC is contacted with a MAChR antagonist/inverse agonist/partial agonist and H3 antagonist/inverse agonist/partial agonist at a “cell effective concentration” to form an expanded population of OPCs in the neural tissue.

A “cell effective concentration” is the minimum concentration of the compound that induces at least an 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more in gene expression and/or about a 1.5-fold increase in number of oligodendrocytes in a OPC Differentiation Assay compared to a vehicle control.

In some embodiments, the OPC is contacted in vitro with the compound(s) at the “cell effective concentration”, such as for example, in a cell culture (and then implanted into the subjects neural tissue, e.g. the centra; nervosu system (CNS)). In other embodiments, the OPC is contacted with the compound(s) at the “cell effective concentration”, in situ (i.e., within the neural tissue, e.g. CNS) In other embodiments, the OPC is contacted with the compound(s) at 2, 3, 4, 5, 10, 20, 50, 100, 200, 300, 400, 500, 600, 700, 800, 900, 1000-fold more than the “cell effective concentration”, in situ.

Alternatively, demyelinating disease or disorders or reduced neuronal function is treated by administering the compound(s) at the “formulation effective concentration”. A “formulation effective concentration” is a higher concentration than the “cell effective formulation”. For example, the “formulation effective concentration” is at least about 10 to 5000 fold higher than the “cell effective concentration”, or about 20, 100, 250, 500, 750, 1000, 1250, 1500, 1750, 2000 fold higher than the “cell effective concentration”, or about 100, 200, 300, 400, 500, 600, 700, 800, 900 1000, 2000. 3000, 4000, 5000 fold higher than the “cell effective concentration”.

Alternatively, demyelinating disease or disorders or reduced neuronal function is treated by administering the compound(s) at a set daily dose.

The compound(s) are formulated at the “cell effective concentration” and the “formulation effective concentration” as described supra.

In some embodiments, the “cell effective concentration” of MAChR antagonist/inverse agonist/partial agonist is at a concentration of about 1 nM to 100,000 nM, about 10 nM to 10,000 nM, about 100 nM to 1,000 nM, about 1 nM to 10 nM, about 10 nM to 100 nM, about 100 nM to 1,000 nM, about 1,000 nM to 10,000 nM, or about 10,000 nM to 100,000 nM.

In some embodiments, the “formulation effective concentration” of MAChR antagonist/inverse agonist/partial agonist is at a concentration of about 1 nM to 1,000,000 μM, about 10 nM to 100,000 μM, about 100 nM, to 10,000 μM, about 1 nM to 10 nM, about 10 nM to 100 nM, about 100 nM to 1,000 nM, about 1,000 nM to 10,000 nM, about 10,000 nM to 100,000 nM, about 100 μM to 1,000 μM, about 1,000 μM to 10,000 μM, about 10,000 μM to 100,000 μM, or about 100,000 μM to 1,000,000 μM.

In some embodiment the MAChR antagonist/inverse agonist/partial agonist is administered to the subject systemically at a daily dose of about 0.01 mg to 10,000 mg/day, about 0.1 mg to 1,000 mg/day, about 1 mg to 250 mg/day, about 0.01 mg to 0.1 mg/day, about 0.1 mg to 1 mg/day, about 1 mg to 10 mg/day, about 10 mg to 100 mg/day, about 100 mg to 1,000 mg/day, or about 1,000 mg to 10,000 mg/day.

In some embodiments, MAChR antagonist/inverse agonist/partial agonist is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration. In some embodiments, compound administered to the subject at about 0.01×. 0.1×, 2×, 3×, 5× or 10×, relative to an FDA approved concentration.

In some embodiments, the MAChR antagonist/inverse agonist/partial agonist is benztropine and is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1000 μM in the cerebrospinal fluid (CSF).

Preferably, the benztropine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF.

In some embodiments, the MAChR antagonist/inverse agonist/partial agonist is benztropine and is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM.

Preferably, the benztropine is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is benztropine and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 25 mg/day, about 25 mg to 50 mg/day, about 50 mg to 100 mg/day, or about 100 mg to 500 mg/day.

In some embodiments, the MAChR antagonist/inverse agonist/partial agonistis benztropine and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, MAChR antagonist/inverse agonist/partial agonist is benztropine and is administered to the subject at about 0.01×. 0.1×, 1×, 2×, 3×, 4×, 5× or 10×, relative to an FDA approved concentration. A benztropine FDA approved concentration is for example the concentration listed on Table 1, column titled “Human Dosage”.

In some embodiments, the MAChR antagonist/inverse agonist/partial agonist is clemastine and is administered for example to a neuronal in amount sufficient to achieve a concentration of about 0.1 nM to 1,000 uM, about 1 nM to 100 uM, about 10 nM to 10 uM, about about 0.1 nM to 1 nM, about 1 nM to 10 nM, about 10 nM to 100 nM, about 100 nM to 1,000 nM, 1 μM to 10 μM, about 10 μM to 100 μM or about 100 μM to 1,000 μM in CSF.

Preferably, the clemastine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the clemastine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is clemastine and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 500 mg/day.

In some embodiments, the MAChR antagonist/inverse agonist/partial agonist is clemastine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, MAChR antagonist/inverse agonist/partial agonist is clemastine and is administered to the subject at about 0.01×. 0.1×, 2×, 3×, 4×, 5× or 10×, relative to an FDA approved concentration. A clemastine approved concentration is for example the concentration listed on Table 1, column titled “Human Dosage”.

In some embodiments, the MAChR antagonist/inverse agonist/partial agonist is oxybutynin and is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM the CSF.

Preferably, the oxybutynin is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, 30 μM or about 50 μM in the neural tissue

In some embodiments, the MAChR antagonist/inverse agonist/partial agonist is oxybutynin and is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

Preferably, the oxybutynin is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is oxybutynin and is administered to a subject systemically at a daily dose of about 0.01 mg to 1,000 mg/day about 0.1 mg to 100 mg/day, about 1 mg to 100 mg/day, about 5 mg to 30 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 20 mg/day, about 20 mg to 30 mg/day, about 30 mg to 50 mg/day, about 50 mg to 100 mg/day, about 100 mg to 250 mg/day, or about 250 mg to 500 mg/day.

In some embodiments, the MAChR antagonist/inverse agonist/partial agonistis oxybutynin and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, MAChR antagonist/inverse agonist/partial agonist is oxybutynin and is administered to the subject at about 0.01×. 0.1×, 1×, 2×, 3×, 4×, 5× or 10×, relative to an FDA approved concentration. An oxybutynin FDA approved concentration is for example the concentration listed on Table 1, column titled “Human Dosage”.

In some embodiments, the MAChR antagonist/inverse agonist/partial agonist is pentoxyverine and is administered for example to a neuronal in amount sufficient to achieve a concentration of about 0.1 nM to 1,000 uM, about 1 nM to 100 uM, about 10 nM to 100 uM, about 100 nM to 10 uM, about 0.1 nM to 1 nM, about 1 nM to 10 nM, about 10 nM to 100 nM, about 100 nM to 1,000 nM, 1 μM to 10 μM, about 10 μM to 100 μM, or about 100 μM to 1,000 μM in CSF.

Preferably, the pentoxyverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, 50 μM, or 100 μM in CSF.

In some embodiments, the MAChR antagonist/inverse agonist/partial agonist is pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pentoxyverine is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is pentoxyverine and is administered to a subject systemically at a daily dose of about 0.1 mg to 10,000 mg/day, about 1 mg to 1,000 mg/day, about 5 mg to 500 mg/day, about 10 mg to 250 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 250 mg/day, about 250 mg to 500 mg/day, about 500 mg to 1,000 mg/day or about 1,000 mg to 10,000 mg/day.

In some embodiments, the MAChR antagonist/inverse agonist/partial agonist is pentoxyverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, MAChR antagonist/inverse agonist/partial agonist is pentoxyverine and is administered to the subject at about 0.01×. 0.1×, 2×, 3×, 4×, 5× or 10×, relative to an FDA approved concentration. A pentoxyverine approved concentration is for example the concentration listed on Table 1, column titled “Human Dosage”.

In some embodiments, the MAChR antagonist/inverse agonist/partial agonist is propiverine and is administered for example to a neuronal in amount sufficient to achieve a concentration of about 0.1 nM to 1000 uM, about 1 nM to 100 uM, about 10 nM to 10 uM, about 0.01 nM to 0.1 nM, about 0.1 nM to 1 nM, about 1 nM to 10 nM, about 10 nM to 100 nM, about 100 nM to 1,000 nM, about 1 μM to 10 μM, 10 μM to 100 μM, or about 100 μM to 1000 μM in CSF.

Preferably, the propiverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the propiverine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM.

In some embodiments, the MAChR antagonist/inverse agonist/partial agonist is propiverine and is administered to a subject systemically at a daily dose of about 0.01 mg to 1000 mg/day, about 0.1 mg to 100 mg/day, about 1 mg to 50 mg/day, about 1 mg to 25 mg/day, about 1 mg to 10 mg/day, about 1 mg to 5 mg/day, about 5 mg to 45 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 to 10 mg/day, about 10 mg to 25 mg/day, about 25 mg to 50 mg/day, about 50 mg to 100 mg/day, about 100 mg to 250 mg/day or about 250 mg to 500 mg/day.

In some embodiments, the MAChR antagonist/inverse agonist/partial agonist is propiverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, MAChR antagonist/inverse agonist/partial agonist is propiverine and is administered to the subject at about 0.01×. 0.1×, 2×, 3×, 4×, 5× or 10×, relative to an FDA approved concentration. A propiverine approved concentration is for example the concentration listed on Table 1, column titled “Human Dosage”.

In some embodiments, the “cell effective concentration” of H3 antagonist/inverse agonist/partial agonist is at a concentration of about 0.1 nM to 100,000 nM, about 1 nM to 10,000 nM, about 10 nM to 1,000 nM, about 0.1 nM to 1 nM, about 1 nM to 10 nM, about 10 nM to 100 nM, about 100 nM to 1,000 nM, about 1,000 nM to 10,000 nM, or about 10,000 nM to 100,000 nM.

In some embodiments, the “formulation effective concentration” of H3 antagonist/inverse agonist/partial agonist is at a concentration of about 1 nM to 100,000 μM, about 10 nM to 10,000 μM, about 100 nM, to 1,000 μM, about 1 nM to 10 nM, about 10 nM to 100 nM, about 100 nM to 1,000 nM, about 1,000 nM to 10,000 nM, about 10,000 nM to 100,000 nM, about 100 μM to 1,000 μM, about 1,000 μM to 10,000 μM, or about 10,000 μM to 100,000 μM.

In some embodiment the H3 antagonist/inverse agonist/partial agonist is administered to the subject systemically at a daily dose of about 0.001 mg to 10,000 mg/day, about 0.01 mg to 1,000 mg/day, about 0.1 mg to 100 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, about 100 mg to 1,000 mg/day, or about 1,000 mg to 10,000 mg/day.

In some embodiments, H3 antagonist/inverse agonist/partial agonist is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration. In some embodiments, compound administered to the subject at about 0.01×. 0.1×, 2×, 3×, 5× or 10×, relative to an FDA approved concentration.

In some embodiments, the H3 antagonist/inverse agonist/partial agonist is ABT-288 and is administered for example to a neural cell in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the ABT-288 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, about 50 μM, or about 100 μM in CSF.

In some embodiments, the H3 antagonist/inverse agonist/partial agonist is ABT-288 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the ABT-288 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the H3 antagonist/inverse agonist/partial agonist is ABT-288 and is administered to a subject systemically at a daily dose of about 0.01 mg to 1,000 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1,000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 100 mg/day, about 100 mg to 500 mg/day, or about 500 mg to 1000 mg/day.

In some embodiments, the H3 antagonist/inverse agonist/partial agonist is ABT-288 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the H3 antagonist/inverse agonist/partial agonist is ABT-288 and is administered to the subject at about 0.01×. 0.1×, 1×, 2×, 3×, 4×, 5× or 10×, relative to an FDA approved concentration. An ABT-288 FDA approved concentration is for example the concentration listed on Table 12, column titled “Human Dosage”.

In some embodiments, the H3 antagonist/inverse agonist/partial agonist is Bavisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the Bavisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the H3 antagonist/inverse agonist/partial agonist is Bavisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the Bavisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the H3 antagonist/inverse agonist/partial agonist is Bavisant and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 500 mg/day, or about 500 mg to 1000 mg/day.

In some embodiments, the H3 antagonist/inverse agonist/partial agonist is Bavisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the H3 antagonist/inverse agonist/partial agonist is Bavisant and is administered to the subject at about 0.01×. 0.1×, 2×, 3×, 4×, 5× or 10×, relative to an FDA approved concentration. A Bavisant approved concentration is for example the concentration listed on Table 2, column titled “Human Dosage”.

In some embodiments, the H3 antagonist/inverse agonist/partial agonist is GSK239512 and is administered for example to a neural cell in amount sufficient to achieve a concentration of about 0.001 nM to 1 mM, about 0.01 nM to 100 μM, about 0.1 nM to 10 μM, about 1 nM to 1 μM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1,000 μM in the CSF.

Preferably, the GSK239512 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the neural tissue

In some embodiments, the H3 antagonist/inverse agonist/partial agonist is GSK239512 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the GSK239512 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the H3 antagonist/inverse agonist/partial agonist is GSK239512 and is administered to a subject systemically at a daily dose of about 0.001 mg to 1,000 mg/day about 0.01 mg to 100 mg/day, about 0.1 mg to 10 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, about 100 mg to 1,000 mg/day, or about 0.01 mg to 0.08 mg/day.

In some embodiments, the H3 antagonist/inverse agonist/partial agonist is GSK239512 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the H3 antagonist/inverse agonist/partial agonist is GSK239512 and is administered to the subject at about 0.01×. 0.1×, 1×, 2×, 3×, 4×, 5× or 10×, relative to an FDA approved concentration. A GSK239512 FDA approved concentration is for example the concentration listed on Table 2, column titled “Human Dosage”.

In some embodiments, the H3 antagonist/inverse agonist/partial agonist is Irdabisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM.

Preferably, the Irdabisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the H3 antagonist/inverse agonist/partial agonist is Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the Irdabisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the H3 antagonist/inverse agonist/partial agonist is Irdabisant and is administered to a subject systemically at a daily dose of about 0.001 mg to 1,000 mg/day, about 0.01 mg to 100 mg/day, 0.1 mg to 10 mg/day, about 0.02 mg to 5 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, or about 100 mg to 1,000 mg/day.

In some embodiments, the H3 antagonist/inverse agonist/partial agonist is Irdabisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the H3 antagonist/inverse agonist/partial agonist is Irdabisant and is administered to the subject at about 0.01×. 0.1×, 2×, 3×, 4×, 5× or 10×, relative to an FDA approved concentration. An Irdabisant approved concentration is for example the concentration listed on Table 2, column titled “Human Dosage”.

In some embodiments, the H3 antagonist/inverse agonist/partial agonist is MK-0249 and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the MK-0249 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the H3 antagonist/inverse agonist/partial agonist is MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the MK-0249 is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the H3 antagonist/inverse agonist/partial agonist is MK-0249 and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, or about 50 mg to 100 mg/day, about 100 mg to 500 mg/day, or about 500 mg to 1000 mg/day.

In some embodiments, the H3 antagonist/inverse agonist/partial agonist is MK-0249 and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the H3 antagonist/inverse agonist/partial agonist is MK-0249 and is administered to the subject at about 0.01×. 0.1×, 2×, 3×, 4×, 5× or 10×, relative to an FDA approved concentration. A MK-0249 approved concentration is for example the concentration listed on Table 2, column titled “Human Dosage”.

In some embodiments, the H3 antagonist/inverse agonist/partial agonist is pitolisant and is administered for example to a neuronal in amount sufficient to achieve a concentration of about 0.001 nM to 10 uM, about 0.01 nM to 1 uM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1,000 nM, 1 μM to 10 μM or about 10 μM to 100 μM in CSF.

Preferably, the pitolisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the H3 antagonist/inverse agonist/partial agonist is pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pitolisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the H3 antagonist/inverse agonist/partial agonist is pitolisant and is administered to a subject systemically at a daily dose of about 0.01 mg to 1,000 mg/day, about 0.1 mg to 100 mg/day, about 1 mg to 50 mg/day, about 5 mg to 50 mg/day, about 5 mg to 10 mg/day, about 9 mg to 36 mg/day about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 1,000 mg/day.

In some embodiments, the H3 antagonist/inverse agonist/partial agonist is pitolisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the H3 antagonist/inverse agonist/partial agonist is pitolisant and is administered to the subject at about 0.01×. 0.1×, 2×, 3×, 4×, 5× or 10×, relative to an FDA approved concentration. A pitolisant approved concentration is for example the concentration listed on Table 2, column titled “Human Dosage”.

In some embodiments, the MAChR antagonist/inverse agonist/partial agonist is benztropine and the H3 antagonist/inverse agonist/partial agonist is ABT-288 and is administered for example to a neuronal cell. In some embodiments, benztropine is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1000 μM in the CSF and ABT-288 is administered for example to a neural cell in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the benztropine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF and the ABT-288 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, about 50 μM, or about 100 μM in CSF.

In some embodiments, the MAChR antagonist/inverse agonist/partial agonist is benztropine and the H3 antagonist/inverse agonist/partial agonist is ABT-288 and is administered to a subject. In some embodiments, the MAChR antagonist/inverse agonist/partial agonist is benztropine and is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and the H3 antagonist/inverse agonist/partial agonist is ABT-288 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the benztropine is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the ABT-288 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is benztropine and the the H3 antagonist/inverse agonist/partial agonist is ABT-288 and is administered to a subject systemically. In some embodiments, the MAChR antagonist/inverse agonist/partial agonist is benztropine and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is ABT-288 and is administered to a subject systemically at a daily dose of about 0.01 mg to 1,000 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1,000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 100 mg/day, about 100 mg to 500 mg/day, or about 500 mg to 1000 mg/day.

In some embodiments, the MAChR antagonist/inverse agonist/partial agonist is benztropine and the H3 antagonist/inverse agonist/partial agonist is ABT-288. In some embodiments, the MAChR antagonist/inverse agonist/partial agonist is benztropine and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the H3 antagonist/inverse agonist/partial agonist is ABT-288 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the MAChR antagonist/inverse agonist/partial agonist is benztropine and the H3 antagonist/inverse agonist/partial agonist is ABT-288. In some embodiments, MAChR antagonist/inverse agonist/partial agonist is benztropine and is administered to the subject at about 0.01×. 0.1×, 1×, 2×, 3×, 4×, 5× or 10×, relative to an FDA approved concentration and the H3 antagonist/inverse agonist/partial agonist is ABT-288 and is administered to the subject at about 0.01×. 0.1×, 1×, 2×, 3×, 4×, 5× or 10×, relative to an FDA approved concentration. A benztropine FDA approved concentration is for example the concentration listed on Table 1, column titled “Human Dosage”. An ABT-288 FDA approved concentration is for example the concentration listed on Table 1, column titled “Human Dosage”.

In some embodiments, the MAChR antagonist/inverse agonist/partial agonist is benztropine and the H3 antagonist/inverse agonist/partial agonist is Bavisant and is administered for example to a neuronal cell. In some embodiments, benztropine is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1000 μM in the CSF and Bavisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the benztropine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF and the Bavisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is benztropine and the H3 antagonist/inverse agonist/partial agonist is Bavisant and is administered to a subject. In some embodiments, the MAChR antagonist/inverse agonist/partial agonist is benztropine and is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and the H3 antagonist/inverse agonist/partial agonist is Bavisant and is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the benztropine is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the Bavisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is benztropine and the the H3 antagonist/inverse agonist/partial agonist is Bavisant and is administered to a subject systemically. In some embodiments, the MAChR antagonist/inverse agonist/partial agonist is benztropine and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is Bavisant and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 500 mg/day, or about 500 mg to 1000 mg/day.

In some embodiments, the MAChR antagonist/inverse agonist/partial agonist is benztropine and the H3 antagonist/inverse agonist/partial agonist is Bavisant. In some embodiments, the MAChR antagonist/inverse agonist/partial agonist is benztropine and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the H3 antagonist/inverse agonist/partial agonist is Bavisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is benztropine and the H3 antagonist/inverse agonist/partial agonist is Bavisant. In some embodiments, MAChR antagonist/inverse agonist/partial agonist is benztropine and is administered to the subject at about 0.01×. 0.1×, 1×, 2×, 3×, 4×, 5× or 10×, relative to an FDA approved concentration and the H3 antagonist/inverse agonist/partial agonist is Bavisant and is administered to the subject at about 0.01×. 0.1×, 2×, 3×, 4×, 5× or 10×, relative to an FDA approved concentration. A benztropine FDA approved concentration is for example the concentration listed on Table 1, column titled “Human Dosage”. A Bavisant FDA approved concentration is for example the concentration listed on Table 1, column titled “Human Dosage”.

In some embodiments, the MAChR antagonist/inverse agonist/partial agonist is benztropine and the H3 antagonist/inverse agonist/partial agonist is GSK239512 and is administered for example to a neuronal cell. In some embodiments, benztropine is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1000 μM in the CSF and GSK239512 and is administered for example to a neural cell in amount sufficient to achieve a concentration of about 0.001 nM to 1 mM, about 0.01 nM to 100 μM, about 0.1 nM to 10 μM, about 1 nM to 1 μM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1,000 μM in the CSF.

Preferably, the benztropine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF and the GSK239512 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF.

In some embodiments, the MAChR antagonist/inverse agonist/partial agonist is benztropine and the H3 antagonist/inverse agonist/partial agonist is GSK239512 and is administered to a subject. In some embodiments, the MAChR antagonist/inverse agonist/partial agonist is benztropine and is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and the H3 antagonist/inverse agonist/partial agonist is GSK239512 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the benztropine is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the GSK239512 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is benztropine and the the H3 antagonist/inverse agonist/partial agonist is GSK239512 and is administered to a subject systemically. In some embodiments, the MAChR antagonist/inverse agonist/partial agonist is benztropine and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is GSK239512 and is administered to a subject systemically at a daily dose of about 0.001 mg to 1,000 mg/day about 0.01 mg to 100 mg/day, about 0.1 mg to 10 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, about 100 mg to 1,000 mg/day, or about 0.01 mg to 0.08 mg/day.

In some embodiments, the MAChR antagonist/inverse agonist/partial agonist is benztropine and the H3 antagonist/inverse agonist/partial agonist is GSK239512. In some embodiments, the MAChR antagonist/inverse agonist/partial agonist is benztropine and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the GSK239512 is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the MAChR antagonist/inverse agonist/partial agonist is benztropine and the H3 antagonist/inverse agonist/partial agonist is GSK239512. In some embodiments, MAChR antagonist/inverse agonist/partial agonist is benztropine and is administered to the subject at about 0.01×. 0.1×, 1×, 2×, 3×, 4×, 5× or 10×, relative to an FDA approved concentration and the H3 antagonist/inverse agonist/partial agonist is GSK239512 and is administered to the subject at about 0.01×. 0.1×, 1×, 2×, 3×, 4×, 5× or 10×, relative to an FDA approved concentration. A benztropine FDA approved concentration is for example the concentration listed on Table 1, column titled “Human Dosage”. A GSK239512 FDA approved concentration is for example the concentration listed on Table 1, column titled “Human Dosage”.

In some embodiments, the MAChR antagonist/inverse agonist/partial agonist is benztropine and the H3 antagonist/inverse agonist/partial agonist is Irdabisant and is administered for example to a neuronal cell. In some embodiments, benztropine is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1000 μM in the CSF and Irdabisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in the CSF.

Preferably, the benztropine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF and the Irdabisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is benztropine and the H3 antagonist/inverse agonist/partial agonist is Irdabisant and is administered to a subject. In some embodiments, the MAChR antagonist/inverse agonist/partial agonist is benztropine and is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and the H3 antagonist/inverse agonist/partial agonist is Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the benztropine is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the Irdabisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is benztropine and the the H3 antagonist/inverse agonist/partial agonist is Irdabisant and is administered to a subject systemically. In some embodiments, the MAChR antagonist/inverse agonist/partial agonist is benztropine and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is Irdabisant and is administered to a subject systemically at a daily dose of about 0.001 mg to 1,000 mg/day, about 0.01 mg to 100 mg/day, 0.1 mg to 10 mg/day, about 0.02 mg to 5 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, or about 100 mg to 1,000 mg/day.

In some embodiments, the MAChR antagonist/inverse agonist/partial agonist is benztropine and the H3 antagonist/inverse agonist/partial agonist is Irdabisant. In some embodiments, the MAChR antagonist/inverse agonist/partial agonist is benztropine and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the H3 antagonist/inverse agonist/partial agonist is Irdabisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is benztropine and the H3 antagonist/inverse agonist/partial agonist is Irdabisant. In some embodiments, MAChR antagonist/inverse agonist/partial agonist is benztropine and is administered to the subject at about 0.01×. 0.1×, 1×, 2×, 3×, 4×, 5× or 10×, relative to an FDA approved concentration and the H3 antagonist/inverse agonist/partial agonist is Irdabisant and is administered to the subject at about 0.01×. 0.1×, 2×, 3×, 4×, 5× or 10×, relative to an FDA approved concentration. A benztropine FDA approved concentration is for example the concentration listed on Table 1, column titled “Human Dosage”. An Irdabisant FDA approved concentration is for example the concentration listed on Table 2, column titled “Human Dosage”.

In some embodiments, the MAChR antagonist/inverse agonist/partial agonist is benztropine and the H3 antagonist/inverse agonist/partial agonist is MK-0249 and is administered for example to a neuronal cell. In some embodiments, benztropine is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1000 μM in the CSF and MK-0249 and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in the CSF.

Preferably, the benztropine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF and the MK-0249 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is benztropine and the H3 antagonist/inverse agonist/partial agonist is MK-0249 and is administered to a subject. In some embodiments, the MAChR antagonist/inverse agonist/partial agonist is benztropine and is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and the H3 antagonist/inverse agonist/partial agonist is MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the benztropine is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the MK-0249 is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is benztropine and the the H3 antagonist/inverse agonist/partial agonist is MK-0249 and is administered to a subject systemically. In some embodiments, the MAChR antagonist/inverse agonist/partial agonist is benztropine and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is MK-0249 and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, or about 50 mg to 100 mg/day, about 100 mg to 500 mg/day, or about 500 mg to 1000 mg/day.

In some embodiments, the MAChR antagonist/inverse agonist/partial agonist is benztropine and the H3 antagonist/inverse agonist/partial agonist is MK-0249. In some embodiments, the MAChR antagonist/inverse agonist/partial agonist is benztropine and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the H3 antagonist/inverse agonist/partial agonist is MK-0249 and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is benztropine and the H3 antagonist/inverse agonist/partial agonist is MK-0249. In some embodiments, MAChR antagonist/inverse agonist/partial agonist is benztropine and is administered to the subject at about 0.01×. 0.1×, 1×, 2×, 3×, 4×, 5× or 10×, relative to an FDA approved concentration and the H3 antagonist/inverse agonist/partial agonist is MK-0249 and is administered to the subject at about 0.01×. 0.1×, 2×, 3×, 4×, 5× or 10×, relative to an FDA approved concentration. A benztropine FDA approved concentration is for example the concentration listed on Table 1, column titled “Human Dosage”. A MK-0249 FDA approved concentration is for example the concentration listed on Table 2, column titled “Human Dosage”.

In some embodiments, the MAChR antagonist/inverse agonist/partial agonist is benztropine and the H3 antagonist/inverse agonist/partial agonist is pitolisant and is administered for example to a neuronal cell. In some embodiments, benztropine is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1000 μM in the CSF and pitolisant and is administered for example to a neuronal in amount sufficient to achieve a concentration of about 0.001 nM to 10 uM, about 0.01 nM to 1 uM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1,000 nM, 1 μM to 10 μM or about 10 μM to 100 μM in CSF.

Preferably, the benztropine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF and the pitolisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is benztropine and the H3 antagonist/inverse agonist/partial agonist is pitolisant and is administered to a subject. In some embodiments, the MAChR antagonist/inverse agonist/partial agonist is benztropine and is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and the H3 antagonist/inverse agonist/partial agonist is pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the benztropine is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the pitolisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the MAChR antagonist/inverse agonist/partial agonist is benztropine and the the H3 antagonist/inverse agonist/partial agonist is pitolisant and is administered to a subject systemically. In some embodiments, the MAChR antagonist/inverse agonist/partial agonist is benztropine and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is pitolisant and is administered to a subject systemically at a daily dose of about 0.01 mg to 1,000 mg/day, about 0.1 mg to 100 mg/day, about 1 mg to 50 mg/day, about 5 mg to 50 mg/day, about 5 mg to 10 mg/day, about 9 mg to 36 mg/day about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 1,000 mg/day.

In some embodiments, the MAChR antagonist/inverse agonist/partial agonist is benztropine and the H3 antagonist/inverse agonist/partial agonist is pitolisant. In some embodiments, the MAChR antagonist/inverse agonist/partial agonist is benztropine and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the H3 antagonist/inverse agonist/partial agonist is pitolisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is benztropine and the H3 antagonist/inverse agonist/partial agonist is pitolisant. In some embodiments, MAChR antagonist/inverse agonist/partial agonist is benztropine and is administered to the subject at about 0.01×. 0.1×, 1×, 2×, 3×, 4×, 5× or 10×, relative to an FDA approved concentration and the H3 antagonist/inverse agonist/partial agonist is pitolisant and is administered to the subject at about 0.01×. 0.1×, 2×, 3×, 4×, 5× or 10×, relative to an FDA approved concentration. A benztropine FDA approved concentration is for example the concentration listed on Table 1, column titled “Human Dosage”. A pitolisant FDA approved concentration is for example the concentration listed on Table 2, column titled “Human Dosage”.

In some embodiments, the MAChR antagonist/inverse agonist/partial agonist is clemastine and the H3 antagonist/inverse agonist/partial agonist is ABT-288 and is administered for example to a neuronal cell. In some embodiments, clemastine and is administered for example to a neuronal in amount sufficient to achieve a concentration of about 0.1 nM to 1,000 uM, about 1 nM to 100 uM, about 10 nM to 10 uM, about about 0.1 nM to 1 nM, about 1 nM to 10 nM, about 10 nM to 100 nM, about 100 nM to 1,000 nM, 1 μM to 10 μM, about 10 μM to 100 μM or about 100 μM to 1,000 μM in CSF and ABT-288 is administered for example to a neural cell in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the clemastine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the ABT-288 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, about 50 μM, or about 100 μM in CSF.

In some embodiments, the MAChR antagonist/inverse agonist/partial agonist is clemastine and the H3 antagonist/inverse agonist/partial agonist is ABT-288 and is administered to a subject. In some embodiments, the MAChR antagonist/inverse agonist/partial agonist is clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is ABT-288 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the clemastine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the ABT-288 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is clemastine and the the H3 antagonist/inverse agonist/partial agonist is ABT-288 and is administered to a subject systemically. In some embodiments, the MAChR antagonist/inverse agonist/partial agonist is MAChR antagonist/inverse agonist/partial agonist is clemastine and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is ABT-288 and is administered to a subject systemically at a daily dose of about 0.01 mg to 1,000 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1,000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 100 mg/day, about 100 mg to 500 mg/day, or about 500 mg to 1000 mg/day.

In some embodiments, the MAChR antagonist/inverse agonist/partial agonist is clemastine and the H3 antagonist/inverse agonist/partial agonist is ABT-288. In some embodiments, the MAChR antagonist/inverse agonist/partial agonist is clemastine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is ABT-288 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the MAChR antagonist/inverse agonist/partial agonist is clemastine and the H3 antagonist/inverse agonist/partial agonist is ABT-288. In some embodiments, MAChR antagonist/inverse agonist/partial agonist is clemastine and is administered to the subject at about 0.01×. 0.1×, 2×, 3×, 4×, 5× or 10×, relative to an FDA approved concentration and the H3 antagonist/inverse agonist/partial agonist is ABT-288 and is administered to the subject at about 0.01×. 0.1×, 1×, 2×, 3×, 4×, 5× or 10×, relative to an FDA approved concentration. A clemastine FDA approved concentration is for example the concentration listed on Table 1, column titled “Human Dosage”. An ABT-288 FDA approved concentration is for example the concentration listed on Table 2, column titled “Human Dosage”.

In some embodiments, the MAChR antagonist/inverse agonist/partial agonist is clemastine and the H3 antagonist/inverse agonist/partial agonist is Bavisant and is administered for example to a neuronal cell. In some embodiments, clemastine and is administered for example to a neuronal in amount sufficient to achieve a concentration of about 0.1 nM to 1,000 uM, about 1 nM to 100 uM, about 10 nM to 10 uM, about about 0.1 nM to 1 nM, about 1 nM to 10 nM, about 10 nM to 100 nM, about 100 nM to 1,000 nM, 1 μM to 10 μM, about 10 μM to 100 μM or about 100 μM to 1,000 μM in CSF and Bavisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the clemastine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the Bavisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is clemastine and the H3 antagonist/inverse agonist/partial agonist is Bavisant and is administered to a subject. In some embodiments, the MAChR antagonist/inverse agonist/partial agonist is clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is Bavisant and is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the clemastine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the Bavisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is clemastine and the the H3 antagonist/inverse agonist/partial agonist is Bavisant and is administered to a subject systemically. In some embodiments, the MAChR antagonist/inverse agonist/partial agonist is MAChR antagonist/inverse agonist/partial agonist is clemastine and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is Bavisant and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 500 mg/day, or about 500 mg to 1000 mg/day.

In some embodiments, the MAChR antagonist/inverse agonist/partial agonist is clemastine and the H3 antagonist/inverse agonist/partial agonist is Bavisant. In some embodiments, the MAChR antagonist/inverse agonist/partial agonist is clemastine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is Bavisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is clemastine and the H3 antagonist/inverse agonist/partial agonist is Bavisant. In some embodiments, MAChR antagonist/inverse agonist/partial agonist is clemastine and is administered to the subject at about 0.01×. 0.1×, 2×, 3×, 4×, 5× or 10×, relative to an FDA approved concentration and the H3 antagonist/inverse agonist/partial agonist is Bavisant and is administered to the subject at about 0.01×. 0.1×, 2×, 3×, 4×, 5× or 10×, relative to an FDA approved concentration. A clemastine FDA approved concentration is for example the concentration listed on Table 1, column titled “Human Dosage”. A Bavisant FDA approved concentration is for example the concentration listed on Table 2, column titled “Human Dosage”.

In some embodiments, the MAChR antagonist/inverse agonist/partial agonist is clemastine and the H3 antagonist/inverse agonist/partial agonist is GSK239512 and is administered for example to a neuronal cell. In some embodiments, clemastine and is administered for example to a neuronal in amount sufficient to achieve a concentration of about 0.1 nM to 1,000 uM, about 1 nM to 100 uM, about 10 nM to 10 uM, about about 0.1 nM to 1 nM, about 1 nM to 10 nM, about 10 nM to 100 nM, about 100 nM to 1,000 nM, 1 μM to 10 μM, about 10 μM to 100 μM or about 100 μM to 1,000 μM in CSF and GSK239512 and is administered for example to a neural cell in amount sufficient to achieve a concentration of about 0.001 nM to 1 mM, about 0.01 nM to 100 μM, about 0.1 nM to 10 μM, about 1 nM to 1 μM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1,000 μM in the CSF.

Preferably, the clemastine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the GSK239512 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF.

In some embodiments, the MAChR antagonist/inverse agonist/partial agonist is clemastine and the H3 antagonist/inverse agonist/partial agonist is GSK239512 and is administered to a subject. In some embodiments, the MAChR antagonist/inverse agonist/partial agonist is clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is GSK239512 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the clemastine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the GSK239512 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is clemastine and the the H3 antagonist/inverse agonist/partial agonist is GSK239512 and is administered to a subject systemically. In some embodiments, the MAChR antagonist/inverse agonist/partial agonist is MAChR antagonist/inverse agonist/partial agonist is clemastine and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is GSK239512 and is administered to a subject systemically at a daily dose of about 0.001 mg to 1,000 mg/day about 0.01 mg to 100 mg/day, about 0.1 mg to 10 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, about 100 mg to 1,000 mg/day, or about 0.01 mg to 0.08 mg/day.

In some embodiments, the MAChR antagonist/inverse agonist/partial agonist is clemastine and the H3 antagonist/inverse agonist/partial agonist is GSK239512. In some embodiments, the MAChR antagonist/inverse agonist/partial agonist is clemastine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the GSK239512 is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the MAChR antagonist/inverse agonist/partial agonist is clemastine and the H3 antagonist/inverse agonist/partial agonist is GSK239512. In some embodiments, MAChR antagonist/inverse agonist/partial agonist is clemastine and is administered to the subject at about 0.01×. 0.1×, 2×, 3×, 4×, 5× or 10×, relative to an FDA approved concentration and the H3 antagonist/inverse agonist/partial agonist is GSK239512 and is administered to the subject at about 0.01×. 0.1×, 1×, 2×, 3×, 4×, 5× or 10×, relative to an FDA approved concentration. A clemastine FDA approved concentration is for example the concentration listed on Table 1, column titled “Human Dosage”. A GSK239512 FDA approved concentration is for example the concentration listed on Table 2, column titled “Human Dosage”.

In some embodiments, the MAChR antagonist/inverse agonist/partial agonist is clemastine and the H3 antagonist/inverse agonist/partial agonist is Irdabisant and is administered for example to a neuronal cell. In some embodiments, clemastine and is administered for example to a neuronal in amount sufficient to achieve a concentration of about 0.1 nM to 1,000 uM, about 1 nM to 100 uM, about 10 nM to 10 uM, about about 0.1 nM to 1 nM, about 1 nM to 10 nM, about 10 nM to 100 nM, about 100 nM to 1,000 nM, 1 μM to 10 μM, about 10 μM to 100 μM or about 100 μM to 1,000 μM in CSF and Irdabisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in the CSF.

Preferably, the clemastine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the Irdabisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is clemastine and the H3 antagonist/inverse agonist/partial agonist is Irdabisant and is administered to a subject. In some embodiments, the MAChR antagonist/inverse agonist/partial agonist is clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the clemastine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the Irdabisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is clemastine and the the H3 antagonist/inverse agonist/partial agonist is Irdabisant and is administered to a subject systemically. In some embodiments, the MAChR antagonist/inverse agonist/partial agonist is MAChR antagonist/inverse agonist/partial agonist is clemastine and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is Irdabisant and is administered to a subject systemically at a daily dose of about 0.001 mg to 1,000 mg/day, about 0.01 mg to 100 mg/day, 0.1 mg to 10 mg/day, about 0.02 mg to 5 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, or about 100 mg to 1,000 mg/day.

In some embodiments, the MAChR antagonist/inverse agonist/partial agonist is clemastine and the H3 antagonist/inverse agonist/partial agonist is Irdabisant. In some embodiments, the MAChR antagonist/inverse agonist/partial agonist is clemastine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is Irdabisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is clemastine and the H3 antagonist/inverse agonist/partial agonist is Irdabisant. In some embodiments, MAChR antagonist/inverse agonist/partial agonist is clemastine and is administered to the subject at about 0.01×. 0.1×, 2×, 3×, 4×, 5× or 10×, relative to an FDA approved concentration and the H3 antagonist/inverse agonist/partial agonist is Irdabisant and is administered to the subject at about 0.01×. 0.1×, 2×, 3×, 4×, 5× or 10×, relative to an FDA approved concentration. A clemastine FDA approved concentration is for example the concentration listed on Table 1, column titled “Human Dosage”. A Irdabisant FDA approved concentration is for example the concentration listed on Table 2, column titled “Human Dosage”.

In some embodiments, the MAChR antagonist/inverse agonist/partial agonist is clemastine and the H3 antagonist/inverse agonist/partial agonist is MK-0249 and is administered for example to a neuronal cell. In some embodiments, clemastine and is administered for example to a neuronal in amount sufficient to achieve a concentration of about 0.1 nM to 1,000 uM, about 1 nM to 100 uM, about 10 nM to 10 uM, about about 0.1 nM to 1 nM, about 1 nM to 10 nM, about 10 nM to 100 nM, about 100 nM to 1,000 nM, 1 μM to 10 μM, about 10 μM to 100 μM or about 100 μM to 1,000 μM in CSF and MK-0249 and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in the CSF.

Preferably, the clemastine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the MK-0249 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is clemastine and the H3 antagonist/inverse agonist/partial agonist is MK-0249 and is administered to a subject. In some embodiments, the MAChR antagonist/inverse agonist/partial agonist is clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the clemastine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the MK-0249 is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is clemastine and the the H3 antagonist/inverse agonist/partial agonist is MK-0249 and is administered to a subject systemically. In some embodiments, the MAChR antagonist/inverse agonist/partial agonist is MAChR antagonist/inverse agonist/partial agonist is clemastine and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is MK-0249 and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, or about 50 mg to 100 mg/day, about 100 mg to 500 mg/day, or about 500 mg to 1000 mg/day.

In some embodiments, the MAChR antagonist/inverse agonist/partial agonist is clemastine and the H3 antagonist/inverse agonist/partial agonist is MK-0249. In some embodiments, the MAChR antagonist/inverse agonist/partial agonist is clemastine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is MK-0249 and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is clemastine and the H3 antagonist/inverse agonist/partial agonist is MK-0249. In some embodiments, MAChR antagonist/inverse agonist/partial agonist is clemastine and is administered to the subject at about 0.01×. 0.1×, 2×, 3×, 4×, 5× or 10×, relative to an FDA approved concentration and the H3 antagonist/inverse agonist/partial agonist is MK-0249 and is administered to the subject at about 0.01×. 0.1×, 2×, 3×, 4×, 5× or 10×, relative to an FDA approved concentration. A clemastine FDA approved concentration is for example the concentration listed on Table 1, column titled “Human Dosage”. A MK-0249 FDA approved concentration is for example the concentration listed on Table 2, column titled “Human Dosage”.

In some embodiments, the MAChR antagonist/inverse agonist/partial agonist is clemastine and the H3 antagonist/inverse agonist/partial agonist is pitolisant and is administered for example to a neuronal cell. In some embodiments, clemastine and is administered for example to a neuronal in amount sufficient to achieve a concentration of about 0.1 nM to 1,000 uM, about 1 nM to 100 uM, about 10 nM to 10 uM, about about 0.1 nM to 1 nM, about 1 nM to 10 nM, about 10 nM to 100 nM, about 100 nM to 1,000 nM, 1 μM to 10 μM, about 10 μM to 100 μM or about 100 μM to 1,000 μM in CSF and pitolisant and is administered for example to a neuronal in amount sufficient to achieve a concentration of about 0.001 nM to 10 uM, about 0.01 nM to 1 uM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1,000 nM, 1 μM to 10 μM or about 10 μM to 100 μM in CSF.

Preferably, the clemastine is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the pitolisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is clemastine and the H3 antagonist/inverse agonist/partial agonist is pitolisant and is administered to a subject. In some embodiments, the MAChR antagonist/inverse agonist/partial agonist is clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the clemastine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the pitolisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the MAChR antagonist/inverse agonist/partial agonist is clemastine and the the H3 antagonist/inverse agonist/partial agonist is pitolisant and is administered to a subject systemically. In some embodiments, the MAChR antagonist/inverse agonist/partial agonist is MAChR antagonist/inverse agonist/partial agonist is clemastine and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 500 mg/day and the H3 antagonist/inverse agonist/partial agonist is pitolisant and is administered to a subject systemically at a daily dose of about 0.01 mg to 1,000 mg/day, about 0.1 mg to 100 mg/day, about 1 mg to 50 mg/day, about 5 mg to 50 mg/day, about 5 mg to 10 mg/day, about 9 mg to 36 mg/day about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 1,000 mg/day.

In some embodiments, the MAChR antagonist/inverse agonist/partial agonist is clemastine and the H3 antagonist/inverse agonist/partial agonist is pitolisant. In some embodiments, the MAChR antagonist/inverse agonist/partial agonist is clemastine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is pitolisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is clemastine and the H3 antagonist/inverse agonist/partial agonist is pitolisant. In some embodiments, MAChR antagonist/inverse agonist/partial agonist is clemastine and is administered to the subject at about 0.01×. 0.1×, 2×, 3×, 4×, 5× or 10×, relative to an FDA approved concentration and the H3 antagonist/inverse agonist/partial agonist is pitolisant and is administered to the subject at about 0.01×. 0.1×, 2×, 3×, 4×, 5× or 10×, relative to an FDA approved concentration. A clemastine FDA approved concentration is for example the concentration listed on Table 1, column titled “Human Dosage”. A pitolisant FDA approved concentration is for example the concentration listed on Table 2, column titled “Human Dosage”.

In some embodiments, the MAChR antagonist/inverse agonist/partial agonist is oxybutynin and the H3 antagonist/inverse agonist/partial agonist is ABT-288 and is administered for example to a neuronal cell. In some embodiments, oxybutynin and is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM the CSF and ABT-288 is administered for example to a neural cell in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the oxybutynin is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, 30 μM or about 50 μM in the CSF and the ABT-288 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, about 50 μM, or about 100 μM in CSF.

In some embodiments, the MAChR antagonist/inverse agonist/partial agonist is oxybutynin and the H3 antagonist/inverse agonist/partial agonist is ABT-288 and is administered to a subject. In some embodiments, the MAChR antagonist/inverse agonist/partial agonist is oxybutynin and is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the H3 antagonist/inverse agonist/partial agonist is ABT-288 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the oxybutynin is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the ABT-288 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is oxybutynin and the the H3 antagonist/inverse agonist/partial agonist is ABT-288 and is administered to a subject systemically. In some embodiments, the MAChR antagonist/inverse agonist/partial agonist is oxybutynin and is administered to a subject systemically at a daily dose of about 0.01 mg to 1,000 mg/day about 0.1 mg to 100 mg/day, about 1 mg to 100 mg/day, about 5 mg to 30 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 20 mg/day, about 20 mg to 30 mg/day, about 30 mg to 50 mg/day, about 50 mg to 100 mg/day, about 100 mg to 250 mg/day, or about 250 mg to 500 mg/day and the H3 antagonist/inverse agonist/partial agonist is ABT-288 and is administered to a subject systemically at a daily dose of about 0.01 mg to 1,000 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1,000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 100 mg/day, about 100 mg to 500 mg/day, or about 500 mg to 1000 mg/day.

In some embodiments, the MAChR antagonist/inverse agonist/partial agonist is oxybutynin and the H3 antagonist/inverse agonist/partial agonist is ABT-288. In some embodiments, the MAChR antagonist/inverse agonist/partial agonist is oxybutynin and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the H3 antagonist/inverse agonist/partial agonist is ABT-288 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the MAChR antagonist/inverse agonist/partial agonist is oxybutynin and the H3 antagonist/inverse agonist/partial agonist is ABT-288. In some embodiments, MAChR antagonist/inverse agonist/partial agonist is oxybutynin and is administered to the subject at about 0.01×. 0.1×, 1×, 2×, 3×, 4×, 5× or 10×, relative to an FDA approved concentration and the H3 antagonist/inverse agonist/partial agonist is ABT-288 and is administered to the subject at about 0.01×. 0.1×, 1×, 2×, 3×, 4×, 5× or 10×, relative to an FDA approved concentration. A oxybutynin FDA approved concentration is for example the concentration listed on Table 1, column titled “Human Dosage”. An ABT-288 FDA approved concentration is for example the concentration listed on Table 2, column titled “Human Dosage”.

In some embodiments, the MAChR antagonist/inverse agonist/partial agonist is oxybutynin and the H3 antagonist/inverse agonist/partial agonist is Bavisant and is administered for example to a neuronal cell. In some embodiments, oxybutynin and is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM the CSF and Bavisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the oxybutynin is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, 30 μM or about 50 μM in the CSF and the Bavisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is oxybutynin and the H3 antagonist/inverse agonist/partial agonist is Bavisant and is administered to a subject. In some embodiments, the MAChR antagonist/inverse agonist/partial agonist is oxybutynin and is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the H3 antagonist/inverse agonist/partial agonist is Bavisant and is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the oxybutynin is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the Bavisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is oxybutynin and the the H3 antagonist/inverse agonist/partial agonist is Bavisant and is administered to a subject systemically. In some embodiments, the MAChR antagonist/inverse agonist/partial agonist is oxybutynin and is administered to a subject systemically at a daily dose of about 0.01 mg to 1,000 mg/day about 0.1 mg to 100 mg/day, about 1 mg to 100 mg/day, about 5 mg to 30 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 20 mg/day, about 20 mg to 30 mg/day, about 30 mg to 50 mg/day, about 50 mg to 100 mg/day, about 100 mg to 250 mg/day, or about 250 mg to 500 mg/day and the H3 antagonist/inverse agonist/partial agonist is Bavisant and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 500 mg/day, or about 500 mg to 1000 mg/day.

In some embodiments, the MAChR antagonist/inverse agonist/partial agonist is oxybutynin and the H3 antagonist/inverse agonist/partial agonist is Bavisant. In some embodiments, the MAChR antagonist/inverse agonist/partial agonist is oxybutynin and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the H3 antagonist/inverse agonist/partial agonist is Bavisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is oxybutynin and the H3 antagonist/inverse agonist/partial agonist is Bavisant. In some embodiments, MAChR antagonist/inverse agonist/partial agonist is oxybutynin and is administered to the subject at about 0.01×. 0.1×, 1×, 2×, 3×, 4×, 5× or 10×, relative to an FDA approved concentration and the H3 antagonist/inverse agonist/partial agonist is Bavisant and is administered to the subject at about 0.01×. 0.1×, 2×, 3×, 4×, 5× or 10×, relative to an FDA approved concentration. A oxybutynin FDA approved concentration is for example the concentration listed on Table 1, column titled “Human Dosage”. A Bavisant FDA approved concentration is for example the concentration listed on Table 2, column titled “Human Dosage”.

In some embodiments, the MAChR antagonist/inverse agonist/partial agonist is oxybutynin and the H3 antagonist/inverse agonist/partial agonist is GSK239512 and is administered for example to a neuronal cell. In some embodiments, oxybutynin and is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM the CSF and GSK239512 and is administered for example to a neural cell in amount sufficient to achieve a concentration of about 0.001 nM to 1 mM, about 0.01 nM to 100 μM, about 0.1 nM to 10 μM, about 1 nM to 1 μM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1,000 μM in the CSF.

Preferably, the oxybutynin is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, 30 μM or about 50 μM in the CSF and the GSK239512 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF.

In some embodiments, the MAChR antagonist/inverse agonist/partial agonist is oxybutynin and the H3 antagonist/inverse agonist/partial agonist is GSK239512 and is administered to a subject. In some embodiments, the MAChR antagonist/inverse agonist/partial agonist is oxybutynin and is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the H3 antagonist/inverse agonist/partial agonist is GSK239512 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the oxybutynin is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the GSK239512 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is oxybutynin and the the H3 antagonist/inverse agonist/partial agonist is GSK239512 and is administered to a subject systemically. In some embodiments, the MAChR antagonist/inverse agonist/partial agonist is oxybutynin and is administered to a subject systemically at a daily dose of about 0.01 mg to 1,000 mg/day about 0.1 mg to 100 mg/day, about 1 mg to 100 mg/day, about 5 mg to 30 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 20 mg/day, about 20 mg to 30 mg/day, about 30 mg to 50 mg/day, about 50 mg to 100 mg/day, about 100 mg to 250 mg/day, or about 250 mg to 500 mg/day and the H3 antagonist/inverse agonist/partial agonist is GSK239512 and is administered to a subject systemically at a daily dose of about 0.001 mg to 1,000 mg/day about 0.01 mg to 100 mg/day, about 0.1 mg to 10 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, about 100 mg to 1,000 mg/day, or about 0.01 mg to 0.08 mg/day.

In some embodiments, the MAChR antagonist/inverse agonist/partial agonist is oxybutynin and the H3 antagonist/inverse agonist/partial agonist is GSK239512. In some embodiments, the MAChR antagonist/inverse agonist/partial agonist is oxybutynin and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the GSK239512 is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the MAChR antagonist/inverse agonist/partial agonist is oxybutynin and the H3 antagonist/inverse agonist/partial agonist is GSK239512. In some embodiments, MAChR antagonist/inverse agonist/partial agonist is oxybutynin and is administered to the subject at about 0.01×. 0.1×, 1×, 2×, 3×, 4×, 5× or 10×, relative to an FDA approved concentration and the H3 antagonist/inverse agonist/partial agonist is GSK239512 and is administered to the subject at about 0.01×. 0.1×, 1×, 2×, 3×, 4×, 5× or 10×, relative to an FDA approved concentration. A oxybutynin FDA approved concentration is for example the concentration listed on Table 1, column titled “Human Dosage”. A GSK239512 FDA approved concentration is for example the concentration listed on Table 2, column titled “Human Dosage”.

In some embodiments, the MAChR antagonist/inverse agonist/partial agonist is oxybutynin and the H3 antagonist/inverse agonist/partial agonist is Irdabisant and is administered for example to a neuronal cell. In some embodiments, oxybutynin and is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM the CSF and Irdabisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in the CSF.

Preferably, the oxybutynin is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, 30 μM or about 50 μM in the CSF and the Irdabisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is oxybutynin and the H3 antagonist/inverse agonist/partial agonist is Irdabisant and is administered to a subject. In some embodiments, the MAChR antagonist/inverse agonist/partial agonist is oxybutynin and is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the H3 antagonist/inverse agonist/partial agonist is Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the oxybutynin is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the Irdabisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is oxybutynin and the the H3 antagonist/inverse agonist/partial agonist is Irdabisant and is administered to a subject systemically. In some embodiments, the MAChR antagonist/inverse agonist/partial agonist is oxybutynin and is administered to a subject systemically at a daily dose of about 0.01 mg to 1,000 mg/day about 0.1 mg to 100 mg/day, about 1 mg to 100 mg/day, about 5 mg to 30 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 20 mg/day, about 20 mg to 30 mg/day, about 30 mg to 50 mg/day, about 50 mg to 100 mg/day, about 100 mg to 250 mg/day, or about 250 mg to 500 mg/day and the H3 antagonist/inverse agonist/partial agonist is Irdabisant and is administered to a subject systemically at a daily dose of about 0.001 mg to 1,000 mg/day, about 0.01 mg to 100 mg/day, 0.1 mg to 10 mg/day, about 0.02 mg to 5 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, or about 100 mg to 1,000 mg/day.

In some embodiments, the MAChR antagonist/inverse agonist/partial agonist is oxybutynin and the H3 antagonist/inverse agonist/partial agonist is Irdabisant. In some embodiments, the MAChR antagonist/inverse agonist/partial agonist is oxybutynin and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the H3 antagonist/inverse agonist/partial agonist is Irdabisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is oxybutynin and the H3 antagonist/inverse agonist/partial agonist is Irdabisant. In some embodiments, MAChR antagonist/inverse agonist/partial agonist is oxybutynin and is administered to the subject at about 0.01×. 0.1×, 1×, 2×, 3×, 4×, 5× or 10×, relative to an FDA approved concentration and the H3 antagonist/inverse agonist/partial agonist is Irdabisant and is administered to the subject at about 0.01×. 0.1×, 2×, 3×, 4×, 5× or 10×, relative to an FDA approved concentration. A oxybutynin FDA approved concentration is for example the concentration listed on Table 1, column titled “Human Dosage”. A Irdabisant FDA approved concentration is for example the concentration listed on Table 2, column titled “Human Dosage”.

In some embodiments, the MAChR antagonist/inverse agonist/partial agonist is oxybutynin and the H3 antagonist/inverse agonist/partial agonist is MK-0249 and is administered for example to a neuronal cell. In some embodiments, oxybutynin and is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM the CSF and MK-0249 and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in the CSF.

Preferably, the oxybutynin is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, 30 μM or about 50 μM in the CSF and the MK-0249 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is oxybutynin and the H3 antagonist/inverse agonist/partial agonist is MK-0249 and is administered to a subject. In some embodiments, the MAChR antagonist/inverse agonist/partial agonist is oxybutynin and is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the H3 antagonist/inverse agonist/partial agonist is MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the oxybutynin is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the MK-0249 is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is oxybutynin and the the H3 antagonist/inverse agonist/partial agonist is MK-0249 and is administered to a subject systemically. In some embodiments, the MAChR antagonist/inverse agonist/partial agonist is oxybutynin and is administered to a subject systemically at a daily dose of about 0.01 mg to 1,000 mg/day about 0.1 mg to 100 mg/day, about 1 mg to 100 mg/day, about 5 mg to 30 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 20 mg/day, about 20 mg to 30 mg/day, about 30 mg to 50 mg/day, about 50 mg to 100 mg/day, about 100 mg to 250 mg/day, or about 250 mg to 500 mg/day and the H3 antagonist/inverse agonist/partial agonist is MK-0249 and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, or about 50 mg to 100 mg/day, about 100 mg to 500 mg/day, or about 500 mg to 1000 mg/day.

In some embodiments, the MAChR antagonist/inverse agonist/partial agonist is oxybutynin and the H3 antagonist/inverse agonist/partial agonist is MK-0249. In some embodiments, the MAChR antagonist/inverse agonist/partial agonist is oxybutynin and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the H3 antagonist/inverse agonist/partial agonist is MK-0249 and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is oxybutynin and the H3 antagonist/inverse agonist/partial agonist is MK-0249. In some embodiments, MAChR antagonist/inverse agonist/partial agonist is oxybutynin and is administered to the subject at about 0.01×. 0.1×, 1×, 2×, 3×, 4×, 5× or 10×, relative to an FDA approved concentration and the H3 antagonist/inverse agonist/partial agonist is MK-0249 and is administered to the subject at about 0.01×. 0.1×, 2×, 3×, 4×, 5× or 10×, relative to an FDA approved concentration. A oxybutynin FDA approved concentration is for example the concentration listed on Table 1, column titled “Human Dosage”. A MK-0249 FDA approved concentration is for example the concentration listed on Table 2, column titled “Human Dosage”.

In some embodiments, the MAChR antagonist/inverse agonist/partial agonist is oxybutynin and the H3 antagonist/inverse agonist/partial agonist is pitolisant and is administered for example to a neuronal cell. In some embodiments, oxybutynin and is administered for example to a neural cell in amount sufficient to achieve a concentration of about 0.1 nM to 1 mM, about 1 nM to 100 μM, about 10 nM to 10 μM, about 100 nM to 1 μM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM the CSF and pitolisant and is administered for example to a neuronal in amount sufficient to achieve a concentration of about 0.001 nM to 10 uM, about 0.01 nM to 1 uM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1,000 nM, 1 μM to 10 μM or about 10 μM to 100 μM in CSF.

Preferably, the oxybutynin is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, 30 μM or about 50 μM in the CSF and the pitolisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is oxybutynin and the H3 antagonist/inverse agonist/partial agonist is pitolisant and is administered to a subject. In some embodiments, the MAChR antagonist/inverse agonist/partial agonist is oxybutynin and is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the H3 antagonist/inverse agonist/partial agonist is pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the oxybutynin is administered to a subject, at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the pitolisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the MAChR antagonist/inverse agonist/partial agonist is oxybutynin and the the H3 antagonist/inverse agonist/partial agonist is pitolisant and is administered to a subject systemically. In some embodiments, the MAChR antagonist/inverse agonist/partial agonist is oxybutynin and is administered to a subject systemically at a daily dose of about 0.01 mg to 1,000 mg/day about 0.1 mg to 100 mg/day, about 1 mg to 100 mg/day, about 5 mg to 30 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, 5 mg to 10 mg/day, about 10 mg to 20 mg/day, about 20 mg to 30 mg/day, about 30 mg to 50 mg/day, about 50 mg to 100 mg/day, about 100 mg to 250 mg/day, or about 250 mg to 500 mg/day and the H3 antagonist/inverse agonist/partial agonist is pitolisant and is administered to a subject systemically at a daily dose of about 0.01 mg to 1,000 mg/day, about 0.1 mg to 100 mg/day, about 1 mg to 50 mg/day, about 5 mg to 50 mg/day, about 5 mg to 10 mg/day, about 9 mg to 36 mg/day about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 1,000 mg/day.

In some embodiments, the MAChR antagonist/inverse agonist/partial agonist is oxybutynin and the H3 antagonist/inverse agonist/partial agonist is pitolisant. In some embodiments, the MAChR antagonist/inverse agonist/partial agonist is oxybutynin and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration and the H3 antagonist/inverse agonist/partial agonist is pitolisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is oxybutynin and the H3 antagonist/inverse agonist/partial agonist is pitolisant. In some embodiments, MAChR antagonist/inverse agonist/partial agonist is oxybutynin and is administered to the subject at about 0.01×. 0.1×, 1×, 2×, 3×, 4×, 5× or 10×, relative to an FDA approved concentration and the H3 antagonist/inverse agonist/partial agonist is pitolisant and is administered to the subject at about 0.01×. 0.1×, 2×, 3×, 4×, 5× or 10×, relative to an FDA approved concentration. A oxybutynin FDA approved concentration is for example the concentration listed on Table 1, column titled “Human Dosage”. A pitolisant FDA approved concentration is for example the concentration listed on Table 2, column titled “Human Dosage”.

In some embodiments, the MAChR antagonist/inverse agonist/partial agonist is pentoxyverine and the H3 antagonist/inverse agonist/partial agonist is ABT-288 and is administered for example to a neuronal cell. In some embodiments, pentoxyverine and is administered for example to a neuronal in amount sufficient to achieve a concentration of about 0.1 nM to 1,000 uM, about 1 nM to 100 uM, about 10 nM to 100 uM, about 100 nM to 10 uM, about 0.1 nM to 1 nM, about 1 nM to 10 nM, about 10 nM to 100 nM, about 100 nM to 1,000 nM, 1 μM to 10 μM, about 10 μM to 100 μM, or about 100 μM to 1,000 μM in CSF and ABT-288 is administered for example to a neural cell in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the pentoxyverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, 50 μM, or 100 μM in CSF and the ABT-288 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, about 50 μM, or about 100 μM in CSF.

In some embodiments, the MAChR antagonist/inverse agonist/partial agonist is pentoxyverine and the H3 antagonist/inverse agonist/partial agonist is ABT-288 and is administered to a subject. In some embodiments, the MAChR antagonist/inverse agonist/partial agonist is pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is ABT-288 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pentoxyverine is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the ABT-288 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is pentoxyverine and the the H3 antagonist/inverse agonist/partial agonist is ABT-288 and is administered to a subject systemically. In some embodiments, the MAChR antagonist/inverse agonist/partial agonist is pentoxyverine and is administered to a subject systemically at a daily dose of about 0.1 mg to 10,000 mg/day, about 1 mg to 1,000 mg/day, about 5 mg to 500 mg/day, about 10 mg to 250 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 250 mg/day, about 250 mg to 500 mg/day, about 500 mg to 1,000 mg/day or about 1,000 mg to 10,000 mg/day and the H3 antagonist/inverse agonist/partial agonist is ABT-288 and is administered to a subject systemically at a daily dose of about 0.01 mg to 1,000 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1,000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 100 mg/day, about 100 mg to 500 mg/day, or about 500 mg to 1000 mg/day.

In some embodiments, the MAChR antagonist/inverse agonist/partial agonist is pentoxyverine and the H3 antagonist/inverse agonist/partial agonist is ABT-288. In some embodiments, the MAChR antagonist/inverse agonist/partial agonist is pentoxyverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is ABT-288 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the MAChR antagonist/inverse agonist/partial agonist is pentoxyverine and the H3 antagonist/inverse agonist/partial agonist is ABT-288. In some embodiments, MAChR antagonist/inverse agonist/partial agonist is pentoxyverine and is administered to the subject at about 0.01×. 0.1×, 2×, 3×, 4×, 5× or 10×, relative to an FDA approved concentration and the H3 antagonist/inverse agonist/partial agonist is ABT-288 and is administered to the subject at about 0.01×. 0.1×, 1×, 2×, 3×, 4×, 5× or 10×, relative to an FDA approved concentration. A pentoxyverine FDA approved concentration is for example the concentration listed on Table 1, column titled “Human Dosage”. An ABT-288 FDA approved concentration is for example the concentration listed on Table 2, column titled “Human Dosage”.

In some embodiments, the MAChR antagonist/inverse agonist/partial agonist is pentoxyverine and the H3 antagonist/inverse agonist/partial agonist is Bavisant and is administered for example to a neuronal cell. In some embodiments, pentoxyverine and is administered for example to a neuronal in amount sufficient to achieve a concentration of about 0.1 nM to 1,000 uM, about 1 nM to 100 uM, about 10 nM to 100 uM, about 100 nM to 10 uM, about 0.1 nM to 1 nM, about 1 nM to 10 nM, about 10 nM to 100 nM, about 100 nM to 1,000 nM, 1 μM to 10 μM, about 10 μM to 100 μM, or about 100 μM to 1,000 μM in CSF and Bavisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the pentoxyverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, 50 μM, or 100 μM in CSF and the Bavisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is pentoxyverine and the H3 antagonist/inverse agonist/partial agonist is Bavisant and is administered to a subject. In some embodiments, the MAChR antagonist/inverse agonist/partial agonist is pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is Bavisant and is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pentoxyverine is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the Bavisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is pentoxyverine and the the H3 antagonist/inverse agonist/partial agonist is Bavisant and is administered to a subject systemically. In some embodiments, the MAChR antagonist/inverse agonist/partial agonist is pentoxyverine and is administered to a subject systemically at a daily dose of about 0.1 mg to 10,000 mg/day, about 1 mg to 1,000 mg/day, about 5 mg to 500 mg/day, about 10 mg to 250 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 250 mg/day, about 250 mg to 500 mg/day, about 500 mg to 1,000 mg/day or about 1,000 mg to 10,000 mg/day and the H3 antagonist/inverse agonist/partial agonist is Bavisant and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 500 mg/day, or about 500 mg to 1000 mg/day.

In some embodiments, the MAChR antagonist/inverse agonist/partial agonist is pentoxyverine and the H3 antagonist/inverse agonist/partial agonist is Bavisant. In some embodiments, the MAChR antagonist/inverse agonist/partial agonist is pentoxyverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is Bavisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is pentoxyverine and the H3 antagonist/inverse agonist/partial agonist is Bavisant. In some embodiments, MAChR antagonist/inverse agonist/partial agonist is pentoxyverine and is administered to the subject at about 0.01×. 0.1×, 2×, 3×, 4×, 5× or 10×, relative to an FDA approved concentration and the H3 antagonist/inverse agonist/partial agonist is Bavisant and is administered to the subject at about 0.01×. 0.1×, 2×, 3×, 4×, 5× or 10×, relative to an FDA approved concentration. A pentoxyverine FDA approved concentration is for example the concentration listed on Table 1, column titled “Human Dosage”. A Bavisant FDA approved concentration is for example the concentration listed on Table 2, column titled “Human Dosage”.

In some embodiments, the MAChR antagonist/inverse agonist/partial agonist is pentoxyverine and the H3 antagonist/inverse agonist/partial agonist is GSK239512 and is administered for example to a neuronal cell. In some embodiments, pentoxyverine and is administered for example to a neuronal in amount sufficient to achieve a concentration of about 0.1 nM to 1,000 uM, about 1 nM to 100 uM, about 10 nM to 100 uM, about 100 nM to 10 uM, about 0.1 nM to 1 nM, about 1 nM to 10 nM, about 10 nM to 100 nM, about 100 nM to 1,000 nM, 1 μM to 10 μM, about 10 μM to 100 μM, or about 100 μM to 1,000 μM in CSF and GSK239512 and is administered for example to a neural cell in amount sufficient to achieve a concentration of about 0.001 nM to 1 mM, about 0.01 nM to 100 μM, about 0.1 nM to 10 μM, about 1 nM to 1 μM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1,000 μM in the CSF.

Preferably, the pentoxyverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, 50 μM, or 100 μM in CSF and the GSK239512 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF.

In some embodiments, the MAChR antagonist/inverse agonist/partial agonist is pentoxyverine and the H3 antagonist/inverse agonist/partial agonist is GSK239512 and is administered to a subject. In some embodiments, the MAChR antagonist/inverse agonist/partial agonist is pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is GSK239512 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pentoxyverine is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the GSK239512 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is pentoxyverine and the the H3 antagonist/inverse agonist/partial agonist is GSK239512 and is administered to a subject systemically. In some embodiments, the MAChR antagonist/inverse agonist/partial agonist is pentoxyverine and is administered to a subject systemically at a daily dose of about 0.1 mg to 10,000 mg/day, about 1 mg to 1,000 mg/day, about 5 mg to 500 mg/day, about 10 mg to 250 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 250 mg/day, about 250 mg to 500 mg/day, about 500 mg to 1,000 mg/day or about 1,000 mg to 10,000 mg/day and the H3 antagonist/inverse agonist/partial agonist is GSK239512 and is administered to a subject systemically at a daily dose of about 0.001 mg to 1,000 mg/day about 0.01 mg to 100 mg/day, about 0.1 mg to 10 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, about 100 mg to 1,000 mg/day, or about 0.01 mg to 0.08 mg/day.

In some embodiments, the MAChR antagonist/inverse agonist/partial agonist is pentoxyverine and the H3 antagonist/inverse agonist/partial agonist is GSK239512. In some embodiments, the MAChR antagonist/inverse agonist/partial agonist is pentoxyverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the GSK239512 is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the MAChR antagonist/inverse agonist/partial agonist is pentoxyverine and the H3 antagonist/inverse agonist/partial agonist is GSK239512. In some embodiments, MAChR antagonist/inverse agonist/partial agonist is pentoxyverine and is administered to the subject at about 0.01×. 0.1×, 2×, 3×, 4×, 5× or 10×, relative to an FDA approved concentration and the H3 antagonist/inverse agonist/partial agonist is GSK239512 and is administered to the subject at about 0.01×. 0.1×, 1×, 2×, 3×, 4×, 5× or 10×, relative to an FDA approved concentration. A pentoxyverine FDA approved concentration is for example the concentration listed on Table 1, column titled “Human Dosage”. A GSK239512 FDA approved concentration is for example the concentration listed on Table 2, column titled “Human Dosage”.

In some embodiments, the MAChR antagonist/inverse agonist/partial agonist is pentoxyverine and the H3 antagonist/inverse agonist/partial agonist is Irdabisant and is administered for example to a neuronal cell. In some embodiments, pentoxyverine and is administered for example to a neuronal in amount sufficient to achieve a concentration of about 0.1 nM to 1,000 uM, about 1 nM to 100 uM, about 10 nM to 100 uM, about 100 nM to 10 uM, about 0.1 nM to 1 nM, about 1 nM to 10 nM, about 10 nM to 100 nM, about 100 nM to 1,000 nM, 1 μM to 10 μM, about 10 μM to 100 μM, or about 100 μM to 1,000 μM in CSF and Irdabisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in the CSF.

Preferably, the pentoxyverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, 50 μM, or 100 μM in CSF and the Irdabisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is pentoxyverine and the H3 antagonist/inverse agonist/partial agonist is Irdabisant and is administered to a subject. In some embodiments, the MAChR antagonist/inverse agonist/partial agonist is pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pentoxyverine is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the Irdabisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is pentoxyverine and the the H3 antagonist/inverse agonist/partial agonist is Irdabisant and is administered to a subject systemically. In some embodiments, the MAChR antagonist/inverse agonist/partial agonist is pentoxyverine and is administered to a subject systemically at a daily dose of about 0.1 mg to 10,000 mg/day, about 1 mg to 1,000 mg/day, about 5 mg to 500 mg/day, about 10 mg to 250 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 250 mg/day, about 250 mg to 500 mg/day, about 500 mg to 1,000 mg/day or about 1,000 mg to 10,000 mg/day and the H3 antagonist/inverse agonist/partial agonist is Irdabisant and is administered to a subject systemically at a daily dose of about 0.001 mg to 1,000 mg/day, about 0.01 mg to 100 mg/day, 0.1 mg to 10 mg/day, about 0.02 mg to 5 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, or about 100 mg to 1,000 mg/day.

In some embodiments, the MAChR antagonist/inverse agonist/partial agonist is pentoxyverine and the H3 antagonist/inverse agonist/partial agonist is Irdabisant. In some embodiments, the MAChR antagonist/inverse agonist/partial agonist is pentoxyverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is Irdabisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is pentoxyverine and the H3 antagonist/inverse agonist/partial agonist is Irdabisant. In some embodiments, MAChR antagonist/inverse agonist/partial agonist is pentoxyverine and is administered to the subject at about 0.01×. 0.1×, 2×, 3×, 4×, 5× or 10×, relative to an FDA approved concentration and the H3 antagonist/inverse agonist/partial agonist is Irdabisant and is administered to the subject at about 0.01×. 0.1×, 2×, 3×, 4×, 5× or 10×, relative to an FDA approved concentration. A pentoxyverine FDA approved concentration is for example the concentration listed on Table 1, column titled “Human Dosage”. An Irdabisant FDA approved concentration is for example the concentration listed on Table 2, column titled “Human Dosage”.

In some embodiments, the MAChR antagonist/inverse agonist/partial agonist is pentoxyverine and the H3 antagonist/inverse agonist/partial agonist is MK-0249 and is administered for example to a neuronal cell. In some embodiments, pentoxyverine and is administered for example to a neuronal in amount sufficient to achieve a concentration of about 0.1 nM to 1,000 uM, about 1 nM to 100 uM, about 10 nM to 100 uM, about 100 nM to 10 uM, about 0.1 nM to 1 nM, about 1 nM to 10 nM, about 10 nM to 100 nM, about 100 nM to 1,000 nM, 1 μM to 10 μM, about 10 μM to 100 μM, or about 100 μM to 1,000 μM in CSF and MK-0249 and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in the CSF.

Preferably, the pentoxyverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, 50 μM, or 100 μM in CSF and the MK-0249 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is pentoxyverine and the H3 antagonist/inverse agonist/partial agonist is MK-0249 and is administered to a subject. In some embodiments, the MAChR antagonist/inverse agonist/partial agonist is pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pentoxyverine is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the MK-0249 is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is pentoxyverine and the the H3 antagonist/inverse agonist/partial agonist is MK-0249 and is administered to a subject systemically. In some embodiments, the MAChR antagonist/inverse agonist/partial agonist is pentoxyverine and is administered to a subject systemically at a daily dose of about 0.1 mg to 10,000 mg/day, about 1 mg to 1,000 mg/day, about 5 mg to 500 mg/day, about 10 mg to 250 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 250 mg/day, about 250 mg to 500 mg/day, about 500 mg to 1,000 mg/day or about 1,000 mg to 10,000 mg/day and the H3 antagonist/inverse agonist/partial agonist is MK-0249 and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, or about 50 mg to 100 mg/day, about 100 mg to 500 mg/day, or about 500 mg to 1000 mg/day.

In some embodiments, the MAChR antagonist/inverse agonist/partial agonist is pentoxyverine and the H3 antagonist/inverse agonist/partial agonist is MK-0249. In some embodiments, the MAChR antagonist/inverse agonist/partial agonist is pentoxyverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is MK-0249 and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is pentoxyverine and the H3 antagonist/inverse agonist/partial agonist is MK-0249. In some embodiments, MAChR antagonist/inverse agonist/partial agonist is pentoxyverine and is administered to the subject at about 0.01×. 0.1×, 2×, 3×, 4×, 5× or 10×, relative to an FDA approved concentration and the H3 antagonist/inverse agonist/partial agonist is MK-0249 and is administered to the subject at about 0.01×. 0.1×, 2×, 3×, 4×, 5× or 10×, relative to an FDA approved concentration. A pentoxyverine FDA approved concentration is for example the concentration listed on Table 1, column titled “Human Dosage”. A MK-0249 FDA approved concentration is for example the concentration listed on Table 2, column titled “Human Dosage”.

In some embodiments, the MAChR antagonist/inverse agonist/partial agonist is pentoxyverine and the H3 antagonist/inverse agonist/partial agonist is pitolisant and is administered for example to a neuronal cell. In some embodiments, pentoxyverine and is administered for example to a neuronal in amount sufficient to achieve a concentration of about 0.1 nM to 1,000 uM, about 1 nM to 100 uM, about 10 nM to 100 uM, about 100 nM to 10 uM, about 0.1 nM to 1 nM, about 1 nM to 10 nM, about 10 nM to 100 nM, about 100 nM to 1,000 nM, 1 μM to 10 μM, about 10 μM to 100 μM, or about 100 μM to 1,000 μM in CSF and pitolisant and is administered for example to a neuronal in amount sufficient to achieve a concentration of about 0.001 nM to 10 uM, about 0.01 nM to 1 uM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1,000 nM, 1 μM to 10 μM or about 10 μM to 100 μM in CSF.

Preferably, the pentoxyverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, 50 μM, or 100 μM in CSF and the pitolisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is pentoxyverine and the H3 antagonist/inverse agonist/partial agonist is pitolisant and is administered to a subject. In some embodiments, the MAChR antagonist/inverse agonist/partial agonist is pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pentoxyverine is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the pitolisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the MAChR antagonist/inverse agonist/partial agonist is pentoxyverine and the the H3 antagonist/inverse agonist/partial agonist is pitolisant and is administered to a subject systemically. In some embodiments, the MAChR antagonist/inverse agonist/partial agonist is pentoxyverine and is administered to a subject systemically at a daily dose of about 0.1 mg to 10,000 mg/day, about 1 mg to 1,000 mg/day, about 5 mg to 500 mg/day, about 10 mg to 250 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 250 mg/day, about 250 mg to 500 mg/day, about 500 mg to 1,000 mg/day or about 1,000 mg to 10,000 mg/day and the H3 antagonist/inverse agonist/partial agonist is pitolisant and is administered to a subject systemically at a daily dose of about 0.01 mg to 1,000 mg/day, about 0.1 mg to 100 mg/day, about 1 mg to 50 mg/day, about 5 mg to 50 mg/day, about 5 mg to 10 mg/day, about 9 mg to 36 mg/day about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 1,000 mg/day.

In some embodiments, the MAChR antagonist/inverse agonist/partial agonist is pentoxyverine and the H3 antagonist/inverse agonist/partial agonist is pitolisant. In some embodiments, the MAChR antagonist/inverse agonist/partial agonist is pentoxyverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is pitolisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is pentoxyverine and the H3 antagonist/inverse agonist/partial agonist is pitolisant. In some embodiments, MAChR antagonist/inverse agonist/partial agonist is pentoxyverine and is administered to the subject at about 0.01×. 0.1×, 2×, 3×, 4×, 5× or 10×, relative to an FDA approved concentration and the H3 antagonist/inverse agonist/partial agonist is pitolisant and is administered to the subject at about 0.01×. 0.1×, 2×, 3×, 4×, 5× or 10×, relative to an FDA approved concentration. A pentoxyverine FDA approved concentration is for example the concentration listed on Table 1, column titled “Human Dosage”. A pitolisant FDA approved concentration is for example the concentration listed on Table 2, column titled “Human Dosage”.

In some embodiments, the MAChR antagonist/inverse agonist/partial agonist is propiverine and the H3 antagonist/inverse agonist/partial agonist is ABT-288 and is administered for example to a neuronal cell. In some embodiments, propiverine and is administered for example to a neuronal in amount sufficient to achieve a concentration of about 0.1 nM to 1000 uM, about 1 nM to 100 uM, about 10 nM to 10 uM, about 0.01 nM to 0.1 nM, about 0.1 nM to 1 nM, about 1 nM to 10 nM, about 10 nM to 100 nM, about 100 nM to 1,000 nM, about 1 μM to 10 μM, 10 μM to 100 μM, or about 100 μM to 1000 μM in CSF and ABT-288 is administered for example to a neural cell in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the propiverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the ABT-288 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, about 50 μM, or about 100 μM in CSF.

In some embodiments, the MAChR antagonist/inverse agonist/partial agonist is propiverine and the H3 antagonist/inverse agonist/partial agonist is ABT-288 and is administered to a subject. In some embodiments, the MAChR antagonist/inverse agonist/partial agonist is propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is ABT-288 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the propiverine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and the ABT-288 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is propiverine and the the H3 antagonist/inverse agonist/partial agonist is ABT-288 and is administered to a subject systemically. In some embodiments, the MAChR antagonist/inverse agonist/partial agonist is propiverine and is administered to a subject systemically at a daily dose of about 0.01 mg to 1000 mg/day, about 0.1 mg to 100 mg/day, about 1 mg to 50 mg/day, about 1 mg to 25 mg/day, about 1 mg to 10 mg/day, about 1 mg to 5 mg/day, about 5 mg to 45 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 to 10 mg/day, about 10 mg to 25 mg/day, about 25 mg to 50 mg/day, about 50 mg to 100 mg/day, about 100 mg to 250 mg/day or about 250 mg to 500 mg/day and the H3 antagonist/inverse agonist/partial agonist is ABT-288 and is administered to a subject systemically at a daily dose of about 0.01 mg to 1,000 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 1,000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 100 mg/day, about 100 mg to 500 mg/day, or about 500 mg to 1000 mg/day.

In some embodiments, the MAChR antagonist/inverse agonist/partial agonist is propiverine and the H3 antagonist/inverse agonist/partial agonist is ABT-288. In some embodiments, the MAChR antagonist/inverse agonist/partial agonist is propiverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is ABT-288 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the MAChR antagonist/inverse agonist/partial agonist is propiverine and the H3 antagonist/inverse agonist/partial agonist is ABT-288. In some embodiments, MAChR antagonist/inverse agonist/partial agonist is propiverine and is administered to the subject at about 0.01×. 0.1×, 2×, 3×, 4×, 5× or 10×, relative to an FDA approved concentration and the H3 antagonist/inverse agonist/partial agonist is ABT-288 and is administered to the subject at about 0.01×. 0.1×, 1×, 2×, 3×, 4×, 5× or 10×, relative to an FDA approved concentration. A propiverine FDA approved concentration is for example the concentration listed on Table 1, column titled “Human Dosage”. An ABT-288 FDA approved concentration is for example the concentration listed on Table 2, column titled “Human Dosage”.

In some embodiments, the MAChR antagonist/inverse agonist/partial agonist is propiverine and the H3 antagonist/inverse agonist/partial agonist is Bavisant and is administered for example to a neuronal cell. In some embodiments, propiverine and is administered for example to a neuronal in amount sufficient to achieve a concentration of about 0.1 nM to 1000 uM, about 1 nM to 100 uM, about 10 nM to 10 uM, about 0.01 nM to 0.1 nM, about 0.1 nM to 1 nM, about 1 nM to 10 nM, about 10 nM to 100 nM, about 100 nM to 1,000 nM, about 1 μM to 10 μM, 10 μM to 100 μM, or about 100 μM to 1000 μM in CSF and Bavisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in CSF.

Preferably, the propiverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the Bavisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is propiverine and the H3 antagonist/inverse agonist/partial agonist is Bavisant and is administered to a subject. In some embodiments, the MAChR antagonist/inverse agonist/partial agonist is propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is Bavisant and is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the propiverine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and the Bavisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is propiverine and the the H3 antagonist/inverse agonist/partial agonist is Bavisant and is administered to a subject systemically. In some embodiments, the MAChR antagonist/inverse agonist/partial agonist is propiverine and is administered to a subject systemically at a daily dose of about 0.01 mg to 1000 mg/day, about 0.1 mg to 100 mg/day, about 1 mg to 50 mg/day, about 1 mg to 25 mg/day, about 1 mg to 10 mg/day, about 1 mg to 5 mg/day, about 5 mg to 45 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 to 10 mg/day, about 10 mg to 25 mg/day, about 25 mg to 50 mg/day, about 50 mg to 100 mg/day, about 100 mg to 250 mg/day or about 250 mg to 500 mg/day and the H3 antagonist/inverse agonist/partial agonist is Bavisant and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, about 100 mg to 500 mg/day, or about 500 mg to 1000 mg/day.

In some embodiments, the MAChR antagonist/inverse agonist/partial agonist is propiverine and the H3 antagonist/inverse agonist/partial agonist is Bavisant. In some embodiments, the MAChR antagonist/inverse agonist/partial agonist is propiverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is Bavisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is propiverine and the H3 antagonist/inverse agonist/partial agonist is Bavisant. In some embodiments, MAChR antagonist/inverse agonist/partial agonist is propiverine and is administered to the subject at about 0.01×. 0.1×, 2×, 3×, 4×, 5× or 10×, relative to an FDA approved concentration and the H3 antagonist/inverse agonist/partial agonist is Bavisant and is administered to the subject at about 0.01×. 0.1×, 2×, 3×, 4×, 5× or 10×, relative to an FDA approved concentration. A propiverine FDA approved concentration is for example the concentration listed on Table 1, column titled “Human Dosage”. A Bavisant FDA approved concentration is for example the concentration listed on Table 2, column titled “Human Dosage”.

In some embodiments, the MAChR antagonist/inverse agonist/partial agonist is propiverine and the H3 antagonist/inverse agonist/partial agonist is GSK239512 and is administered for example to a neuronal cell. In some embodiments, propiverine and is administered for example to a neuronal in amount sufficient to achieve a concentration of about 0.1 nM to 1000 uM, about 1 nM to 100 uM, about 10 nM to 10 uM, about 0.01 nM to 0.1 nM, about 0.1 nM to 1 nM, about 1 nM to 10 nM, about 10 nM to 100 nM, about 100 nM to 1,000 nM, about 1 μM to 10 μM, 10 μM to 100 μM, or about 100 μM to 1000 μM in CSF and GSK239512 and is administered for example to a neural cell in amount sufficient to achieve a concentration of about 0.001 nM to 1 mM, about 0.01 nM to 100 μM, about 0.1 nM to 10 μM, about 1 nM to 1 μM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1,000 μM in the CSF.

Preferably, the propiverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the GSK239512 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the CSF.

In some embodiments, the MAChR antagonist/inverse agonist/partial agonist is propiverine and the H3 antagonist/inverse agonist/partial agonist is GSK239512 and is administered to a subject. In some embodiments, the MAChR antagonist/inverse agonist/partial agonist is propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is GSK239512 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the propiverine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and the GSK239512 is administered to a subject, at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the MAChR antagonist/inverse agonist/partial agonist is propiverine and the the H3 antagonist/inverse agonist/partial agonist is GSK239512 and is administered to a subject systemically. In some embodiments, the MAChR antagonist/inverse agonist/partial agonist is propiverine and is administered to a subject systemically at a daily dose of about 0.01 mg to 1000 mg/day, about 0.1 mg to 100 mg/day, about 1 mg to 50 mg/day, about 1 mg to 25 mg/day, about 1 mg to 10 mg/day, about 1 mg to 5 mg/day, about 5 mg to 45 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 to 10 mg/day, about 10 mg to 25 mg/day, about 25 mg to 50 mg/day, about 50 mg to 100 mg/day, about 100 mg to 250 mg/day or about 250 mg to 500 mg/day and the H3 antagonist/inverse agonist/partial agonist is GSK239512 and is administered to a subject systemically at a daily dose of about 0.001 mg to 1,000 mg/day about 0.01 mg to 100 mg/day, about 0.1 mg to 10 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, about 100 mg to 1,000 mg/day, or about 0.01 mg to 0.08 mg/day.

In some embodiments, the MAChR antagonist/inverse agonist/partial agonist is propiverine and the H3 antagonist/inverse agonist/partial agonist is GSK239512. In some embodiments, the MAChR antagonist/inverse agonist/partial agonist is propiverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the GSK239512 is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the MAChR antagonist/inverse agonist/partial agonist is propiverine and the H3 antagonist/inverse agonist/partial agonist is GSK239512. In some embodiments, MAChR antagonist/inverse agonist/partial agonist is propiverine and is administered to the subject at about 0.01×. 0.1×, 2×, 3×, 4×, 5× or 10×, relative to an FDA approved concentration and the H3 antagonist/inverse agonist/partial agonist is GSK239512 and is administered to the subject at about 0.01×. 0.1×, 1×, 2×, 3×, 4×, 5× or 10×, relative to an FDA approved concentration. A propiverine FDA approved concentration is for example the concentration listed on Table 1, column titled “Human Dosage”. A GSK239512 FDA approved concentration is for example the concentration listed on Table 2, column titled “Human Dosage”.

In some embodiments, the MAChR antagonist/inverse agonist/partial agonist is propiverine and the H3 antagonist/inverse agonist/partial agonist is Irdabisant and is administered for example to a neuronal cell. In some embodiments, propiverine and is administered for example to a neuronal in amount sufficient to achieve a concentration of about 0.1 nM to 1000 uM, about 1 nM to 100 uM, about 10 nM to 10 uM, about 0.01 nM to 0.1 nM, about 0.1 nM to 1 nM, about 1 nM to 10 nM, about 10 nM to 100 nM, about 100 nM to 1,000 nM, about 1 μM to 10 μM, 10 μM to 100 μM, or about 100 μM to 1000 μM in CSF and Irdabisant and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in the CSF.

Preferably, the propiverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the Irdabisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is propiverine and the H3 antagonist/inverse agonist/partial agonist is Irdabisant and is administered to a subject. In some embodiments, the MAChR antagonist/inverse agonist/partial agonist is propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the propiverine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and the Irdabisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is propiverine and the the H3 antagonist/inverse agonist/partial agonist is Irdabisant and is administered to a subject systemically. In some embodiments, the MAChR antagonist/inverse agonist/partial agonist is propiverine and is administered to a subject systemically at a daily dose of about 0.01 mg to 1000 mg/day, about 0.1 mg to 100 mg/day, about 1 mg to 50 mg/day, about 1 mg to 25 mg/day, about 1 mg to 10 mg/day, about 1 mg to 5 mg/day, about 5 mg to 45 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 to 10 mg/day, about 10 mg to 25 mg/day, about 25 mg to 50 mg/day, about 50 mg to 100 mg/day, about 100 mg to 250 mg/day or about 250 mg to 500 mg/day and the H3 antagonist/inverse agonist/partial agonist is Irdabisant and is administered to a subject systemically at a daily dose of about 0.001 mg to 1,000 mg/day, about 0.01 mg to 100 mg/day, 0.1 mg to 10 mg/day, about 0.02 mg to 5 mg/day, about 0.001 mg to 0.01 mg/day, about 0.01 mg to 0.1 mg/day, about 0.1 mg to 1 mg/day, about 1 mg to 10 mg/day, about 10 mg to 100 mg/day, or about 100 mg to 1,000 mg/day.

In some embodiments, the MAChR antagonist/inverse agonist/partial agonist is propiverine and the H3 antagonist/inverse agonist/partial agonist is Irdabisant. In some embodiments, the MAChR antagonist/inverse agonist/partial agonist is propiverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is Irdabisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is propiverine and the H3 antagonist/inverse agonist/partial agonist is Irdabisant. In some embodiments, MAChR antagonist/inverse agonist/partial agonist is propiverine and is administered to the subject at about 0.01×. 0.1×, 2×, 3×, 4×, 5× or 10×, relative to an FDA approved concentration and the H3 antagonist/inverse agonist/partial agonist is Irdabisant and is administered to the subject at about 0.01×. 0.1×, 2×, 3×, 4×, 5× or 10×, relative to an FDA approved concentration. A propiverine FDA approved concentration is for example the concentration listed on Table 1, column titled “Human Dosage”. A Irdabisant FDA approved concentration is for example the concentration listed on Table 2 column titled “Human Dosage”.

M some embodiments, the MAChR antagonist/inverse agonist/partial agonist is propiverine and the H3 antagonist/inverse agonist/partial agonist is MK-0249 and is administered for example to a neuronal cell. In some embodiments, propiverine and is administered for example to a neuronal in amount sufficient to achieve a concentration of about 0.1 nM to 1000 uM, about 1 nM to 100 uM, about 10 nM to 10 uM, about 0.01 nM to 0.1 nM, about 0.1 nM to 1 nM, about 1 nM to 10 nM, about 10 nM to 100 nM, about 100 nM to 1,000 nM, about 1 μM to 10 μM, 10 μM to 100 μM, or about 100 μM to 1000 μM in CSF and MK-0249 and is administered for example to a neuronal in amount sufficient to achieve a concentration of about 0.001 nM to 1 mM, about 0.01 nM to 100 μM, about 0.1 nM to 10 μM, about 1 nM to 1 μM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, about 1 μM to 10 μM, about 10 μM to 100 μM or about 100 μM to 1,000 μM in the CSF.

Preferably, the propiverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the MK-0249 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

M some embodiments, the MAChR antagonist/inverse agonist/partial agonist is propiverine and the H3 antagonist/inverse agonist/partial agonist is MK-0249 and is administered to a subject. In some embodiments, the MAChR antagonist/inverse agonist/partial agonist is propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the propiverine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and the MK-0249 is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the MAChR antagonist/inverse agonist/partial agonist is propiverine and the the H3 antagonist/inverse agonist/partial agonist is MK-0249 and is administered to a subject systemically. In some embodiments, the MAChR antagonist/inverse agonist/partial agonist is propiverine and is administered to a subject systemically at a daily dose of about 0.01 mg to 1000 mg/day, about 0.1 mg to 100 mg/day, about 1 mg to 50 mg/day, about 1 mg to 25 mg/day, about 1 mg to 10 mg/day, about 1 mg to 5 mg/day, about 5 mg to 45 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 to 10 mg/day, about 10 mg to 25 mg/day, about 25 mg to 50 mg/day, about 50 mg to 100 mg/day, about 100 mg to 250 mg/day or about 250 mg to 500 mg/day and the H3 antagonist/inverse agonist/partial agonist is MK-0249 and is administered to a subject systemically at a daily dose of about 0.01 mg to 1,000 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, or about 50 mg to 100 mg/day, about 100 mg to 500 mg/day, or about 500 mg to 1000 mg/day.

In some embodiments, the MAChR antagonist/inverse agonist/partial agonist is propiverine and the H3 antagonist/inverse agonist/partial agonist is MK-0249. In some embodiments, the MAChR antagonist/inverse agonist/partial agonist is propiverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is MK-0249 and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is propiverine and the H3 antagonist/inverse agonist/partial agonist is MK-0249. In some embodiments, MAChR antagonist/inverse agonist/partial agonist is propiverine and is administered to the subject at about 0.01×. 0.1×, 2×, 3×, 4×, 5× or 10×, relative to an FDA approved concentration and the H3 antagonist/inverse agonist/partial agonist is MK-0249 and is administered to the subject at about 0.01×. 0.1×, 2×, 3×, 4×, 5× or 10×, relative to an FDA approved concentration. A propiverine FDA approved concentration is for example the concentration listed on Table 1, column titled “Human Dosage”. A MK-0249 FDA approved concentration is for example the concentration listed on Table 2, column titled “Human Dosage”.

In some embodiments, the MAChR antagonist/inverse agonist/partial agonist is propiverine and the H3 antagonist/inverse agonist/partial agonist is pitolisant and is administered for example to a neuronal cell. In some embodiments, propiverine and is administered for example to a neuronal in amount sufficient to achieve a concentration of about 0.1 nM to 1000 uM, about 1 nM to 100 uM, about 10 nM to 10 uM, about 0.01 nM to 0.1 nM, about 0.1 nM to 1 nM, about 1 nM to 10 nM, about 10 nM to 100 nM, about 100 nM to 1,000 nM, about 1 μM to 10 μM, 10 μM to 100 μM, or about 100 μM to 1000 μM in CSF and pitolisant and is administered for example to a neuronal in amount sufficient to achieve a concentration of about 0.001 nM to 10 uM, about 0.01 nM to 1 uM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1,000 nM, 1 μM to 10 μM or about 10 μM to 100 μM in CSF.

Preferably, the propiverine is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF and the pitolisant is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in CSF.

In some embodiments, the MAChR antagonist/inverse agonist/partial agonist is propiverine and the H3 antagonist/inverse agonist/partial agonist is pitolisant and is administered to a subject. In some embodiments, the MAChR antagonist/inverse agonist/partial agonist is propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the H3 antagonist/inverse agonist/partial agonist is pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the propiverine is administered to a subject at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and the pitolisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the MAChR antagonist/inverse agonist/partial agonist is propiverine and the the H3 antagonist/inverse agonist/partial agonist is pitolisant and is administered to a subject systemically. In some embodiments, the MAChR antagonist/inverse agonist/partial agonist is propiverine and is administered to a subject systemically at a daily dose of about 0.01 mg to 1000 mg/day, about 0.1 mg to 100 mg/day, about 1 mg to 50 mg/day, about 1 mg to 25 mg/day, about 1 mg to 10 mg/day, about 1 mg to 5 mg/day, about 5 mg to 45 mg/day, about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 to 10 mg/day, about 10 mg to 25 mg/day, about 25 mg to 50 mg/day, about 50 mg to 100 mg/day, about 100 mg to 250 mg/day or about 250 mg to 500 mg/day and the H3 antagonist/inverse agonist/partial agonist is pitolisant and is administered to a subject systemically at a daily dose of about 0.01 mg to 1,000 mg/day, about 0.1 mg to 100 mg/day, about 1 mg to 50 mg/day, about 5 mg to 50 mg/day, about 5 mg to 10 mg/day, about 9 mg to 36 mg/day about 0.01 mg to 0.1 mg/day, about 0.1 mg to 1 mg/day, about 1 mg to 10 mg/day, about 10 mg to 100 mg/day, about 100 mg to 1000 mg/day, about 0.5 mg to 1 mg/day, about 1 mg to 2 mg/day, about 2 mg to 3 mg/day, about 3 mg to 4 mg/day, about 4 mg to 5 mg/day, about 5 mg to 10 mg/day, about 10 mg to 25 mg/day, about 25 mg to 50 mg/day, about 50 mg to 100 mg/day, or about 100 mg to 1,000 mg/day.

In some embodiments, the MAChR antagonist/inverse agonist/partial agonist is propiverine and the H3 antagonist/inverse agonist/partial agonist is pitolisant. In some embodiments, the MAChR antagonist/inverse agonist/partial agonist is propiverine and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration and the H3 antagonist/inverse agonist/partial agonist is pitolisant and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, the MAChR antagonist/inverse agonist/partial agonist is propiverine and the H3 antagonist/inverse agonist/partial agonist is pitolisant. In some embodiments, MAChR antagonist/inverse agonist/partial agonist is propiverine and is administered to the subject at about 0.01×. 0.1×, 2×, 3×, 4×, 5× or 10×, relative to an FDA approved concentration and the H3 antagonist/inverse agonist/partial agonist is pitolisant and is administered to the subject at about 0.01×. 0.1×, 2×, 3×, 4×, 5× or 10×, relative to an FDA approved concentration. A propiverine FDA approved concentration is for example the concentration listed on Table 1, column titled “Human Dosage”. A pitolisant FDA approved concentration is for example the concentration listed on Table 2, column titled “Human Dosage”.

Some embodiments comprise administering the (i) MAChR antagonist/inverse agonist/partial agonist and (ii) H3 antagonist/inverse agonist/partial agonist together in the same pharmaceutical composition, as described herein. Some embodiments comprise administering the (i) MAChR antagonist/inverse agonist/partial agonist and (ii) H3 antagonist/inverse agonist/partial agonist separately in separate pharmaceutical compositions.

### Pharmaceutical Compositions and Administration

In another aspect, the present disclosure provides pharmaceutical compositions comprising: (i) a pharmaceutically-acceptable carrier and an agent having dual activity as a MAChR antagonist/inverse agonist/partial agonist and a histamine H3 receptor antagonist/inverse agonist/partial agonist or a pharmaceutically-acceptable salt thereof; or (ii) a pharmaceutically-acceptable carrier, a first agent having activity as a MAChR antagonist/inverse agonist/partial agonist and a second agent having activity as a histamine H3 receptor antagonist/inverse agonist/partial agonist or pharmaceutically-acceptable salts thereof.

Exemplary agents for inclusion in the pharmaceutical composition of the invention are described above in Table 1 and 2 above

In some embodiments, the pharmaceutical compositions of the inventions includes an agent of having dual activity as a MAChR antagonist/inverse agonist/partial agonist and a histamine H3 receptor antagonist/inverse agonist/partial agonist is selected from Table 1, including pharmaceutically-acceptable salts thereof. In some embodiments, the agent is not clemastine. In particular embodiments, the pharmaceutical compositions of the inventions includes an agent having activity as a MAChR antagonist/inverse agonist/partial agonist (and in some instances, having little to no activity as a histamine H3 receptor antagonist/inverse agonist/partial agonist) is selected from Table 2. In certain of these and related embodiments, the agent having activity as a MAChR antagonist/inverse agonist/partial agonist (and in some instances, having little to no activity as a histamine H3 receptor antagonist/inverse agonist/partial agonist) are Atropine, Benztropine, Chlorpromazine, Clemastine. Dicyclomine, Diphenylpyraline, Disopyramide, Hyoscyamin, Mepenzolate, Orphenadrine, Oxybutynin, Paroxetine, Pentoxyverine, Piperidolate, Propafenone, Propiverine, Quetiapine, Scopolamine, Solifenacin, Tolterodine, Trihexyphenidyl, or Tripelennamine. In other embodiments, agents having activity as a MAChR antagonist/inverse agonist/partial agonist (and in some instances, having little to no activity as a histamine H3 receptor antagonist/inverse agonist/partial agonist) are benztropine, clemastine, oxybutynin, pentoxyverine, propiverine.

In particular embodiments, the pharmaceutical compositions of the inventions includes an agent having activity as a histamine H3 receptor antagonist/inverse agonist/partial agonist is selected from Table 2 including pharmaceutically-acceptable salts thereof. In some embodiments, the pharmaceutical compositions of the inventions includes ABT-288, Bavisant, GSK239512, Irdabisant, MK-0249 or pitolisant.

In some embodiments, the pharmaceutical composition comprises an MAChR antagonist/inverse agonist/partial agonist that is benztropine at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM.

Preferably, the pharmaceutical composition comprises benztropine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises benztropine at a daily dose of about 0.01 mg to 500 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 25 mg, about 25 mg to 50 mg, about 50 mg to 100 mg, or about 100 mg to 500 mg.

In some embodiments, the pharmaceutical composition comprises an MAChR antagonist/inverse agonist/partial agonist that is clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pharmaceutical composition comprises clemastine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises clemastine at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 500 mg.

In some embodiments, the pharmaceutical composition comprises an MAChR antagonist/inverse agonist/partial agonist is that oxybutynin at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

Preferably, the pharmaceutical composition comprises oxybutynin at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises oxybutynin at a daily dose of about 0.01 mg to 1,000 mg about 0.1 mg to 100 mg, about 1 mg to 100 mg, about 5 mg to 30 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 20 mg, about 20 mg to 30 mg, about 30 mg to 50 mg, about 50 mg to 100 mg, about 100 mg to 250 mg, or about 250 mg to 500 mg.

In some embodiments, the pharmaceutical composition comprises an MAChR antagonist/inverse agonist/partial agonist that is pentoxyverine at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pharmaceutical composition comprises pentoxyverine at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises pentoxyverine at a daily dose of about 0.1 mg to 10,000 mg, about 1 mg to 1,000 mg, about 5 mg to 500 mg, about 10 mg to 250 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 250 mg, about 250 mg to 500 mg, about 500 mg to 1,000 mg or about 1,000 mg to 10,000 mg.

In some embodiments, the pharmaceutical composition comprises an MAChR antagonist/inverse agonist/partial agonist that is propiverine at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pharmaceutical composition comprises propiverine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM.

In some embodiments, the pharmaceutical composition comprises propiverine at a daily dose of about 0.01 mg to 1000 mg, about 0.1 mg to 100 mg, about 1 mg to 50 mg, about 1 mg to 25 mg, about 1 mg to 10 mg, about 1 mg to 5 mg, about 5 mg to 45 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 to 10 mg, about 10 mg to 25 mg, about 25 mg to 50 mg, about 50 mg to 100 mg, about 100 mg to 250 mg or about 250 mg to 500 mg.

In some embodiments, the pharmaceutical composition comprises an H3 antagonist/inverse agonist/partial agonist that is ABT-288 at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises ABT-288 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises ABT-288 at a daily dose of about 0.01 mg to 1,000 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1,000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 100 mg, about 100 mg to 500 mg, or about 500 mg to 1000 mg.

In some embodiments, the pharmaceutical composition comprises an H3 antagonist/inverse agonist/partial agonist that is Bavisant at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises Bavisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises Bavisant at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 500 mg, or about 500 mg to 1000 mg.

In some embodiments, the pharmaceutical composition comprises an H3 antagonist/inverse agonist/partial agonist that is GSK239512 at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises GSK239512 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises GSK239512 at a daily dose of about 0.001 mg to 1,000 mg about 0.01 mg to 100 mg, about 0.1 mg to 10 mg, about 0.001 mg to 0.01 mg, about 0.01 mg to 0.1 mg, about 0.1 mg to 1 mg, about 1 mg to 10 mg, about 10 mg to 100 mg, about 100 mg to 1,000 mg, or about 0.01 mg to 0.08 mg.

In some embodiments, the pharmaceutical composition comprises an H3 antagonist/inverse agonist/partial agonist that is Irdabisant at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises Irdabisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises Irdabisant at a daily dose of about 0.001 mg to 1,000 mg, about 0.01 mg to 100 mg, 0.1 mg to 10 mg, about 0.02 mg to 5 mg, about 0.001 mg to 0.01 mg, about 0.01 mg to 0.1 mg, about 0.1 mg to 1 mg, about 1 mg to 10 mg, about 10 mg to 100 mg, or about 100 mg to 1,000 mg.

In some embodiments, the pharmaceutical composition comprises an H3 antagonist/inverse agonist/partial agonist that is MK-0249 i at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises MK-0249 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises H3 antagonist/inverse agonist/partial agonist is MK-0249 at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, or about 50 mg to 100 mg, about 100 mg to 500 mg, or about 500 mg to 1000 mg.

In some embodiments, the pharmaceutical composition comprises an H3 antagonist/inverse agonist/partial agonist that is pitolisant at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pharmaceutical composition comprises pitolisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the pharmaceutical composition comprises pitolisant at a daily dose of about 0.01 mg to 1,000 mg, about 0.1 mg to 100 mg, about 1 mg to 50 mg, about 5 mg to 50 mg, about 5 mg to 10 mg, about 9 mg to 36 mg about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 1,000 mg.

In some embodiments, the pharmaceutical composition comprises an MAChR antagonist/inverse agonist/partial agonist that is benztropine and an H3 antagonist/inverse agonist/partial agonist that is ABT-288 and is administered to a subject. In some embodiments, benztropine is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and ABT-288 is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises benztropine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and ABT-288 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises benztropine and ABT-288. In some embodiments, the pharmaceutical composition comprises benztropine at a daily dose of about 0.01 mg to 500 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 25 mg, about 25 mg to 50 mg, about 50 mg to 100 mg, or about 100 mg to 500 mg and ABT-288 at a daily dose of about 0.01 mg to 1,000 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1,000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 100 mg, about 100 mg to 500 mg, or about 500 mg to 1000 mg.

In some embodiments, the pharmaceutical composition comprises an MAChR antagonist/inverse agonist/partial agonist that is benztropine and an H3 antagonist/inverse agonist/partial agonist that is Bavisant and is administered to a subject. In some embodiments, benztropine is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and Bavisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises benztropine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and Bavisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

M some embodiments, the pharmaceutical composition comprises benztropine and Bavisant. In some embodiments, the pharmaceutical composition comprises benztropine at a daily dose of about 0.01 mg to 500 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 25 mg, about 25 mg to 50 mg, about 50 mg to 100 mg, or about 100 mg to 500 mg and Bavisant at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 500 mg, or about 500 mg to 1000 mg.

In some embodiments, the pharmaceutical composition comprises an MAChR antagonist/inverse agonist/partial agonist that is benztropine and an H3 antagonist/inverse agonist/partial agonist that is GSK239512 and is administered to a subject. In some embodiments, benztropine is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and GSK239512 is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises benztropine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and GSK239512 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises benztropine and GSK239512. In some embodiments, the pharmaceutical composition comprises benztropine at a daily dose of about 0.01 mg to 500 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 25 mg, about 25 mg to 50 mg, about 50 mg to 100 mg, or about 100 mg to 500 mg and GSK239512 at a daily dose of about 0.001 mg to 1,000 mg about 0.01 mg to 100 mg, about 0.1 mg to 10 mg, about 0.001 mg to 0.01 mg, about 0.01 mg to 0.1 mg, about 0.1 mg to 1 mg, about 1 mg to 10 mg, about 10 mg to 100 mg, about 100 mg to 1,000 mg, or about 0.01 mg to 0.08 mg.

In some embodiments, the pharmaceutical composition comprises an MAChR antagonist/inverse agonist/partial agonist that is benztropine and an H3 antagonist/inverse agonist/partial agonist that is Irdabisant and is administered to a subject. In some embodiments, benztropine is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises benztropine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and Irdabisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises benztropine and Irdabisant. In some embodiments, the pharmaceutical composition comprises benztropine at a daily dose of about 0.01 mg to 500 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 25 mg, about 25 mg to 50 mg, about 50 mg to 100 mg, or about 100 mg to 500 mg and Irdabisant at a daily dose of about 0.001 mg to 1,000 mg, about 0.01 mg to 100 mg, 0.1 mg to 10 mg, about 0.02 mg to 5 mg, about 0.001 mg to 0.01 mg, about 0.01 mg to 0.1 mg, about 0.1 mg to 1 mg, about 1 mg to 10 mg, about 10 mg to 100 mg, or about 100 mg to 1,000 mg.

In some embodiments, the pharmaceutical composition comprises an MAChR antagonist/inverse agonist/partial agonist that is benztropine and an H3 antagonist/inverse agonist/partial agonist that is MK-0249 and is administered to a subject. In some embodiments, benztropine is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises benztropine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and MK- at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises benztropine and MK-0249. In some embodiments, the pharmaceutical composition comprises benztropine at a daily dose of about 0.01 mg to 500 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 25 mg, about 25 mg to 50 mg, about 50 mg to 100 mg, or about 100 mg to 500 mg and MK-0249 a at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, or about 50 mg to 100 mg, about 100 mg to 500 mg, or about 500 mg to 1000 mg.

In some embodiments, the pharmaceutical composition comprises an MAChR antagonist/inverse agonist/partial agonist that is benztropine and an H3 antagonist/inverse agonist/partial agonist that is pitolisant and is administered to a subject. In some embodiments, benztropine is administered to a subject, at a concentration of 0.001 μM to 1,000 mM, about 0.01 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, 1 mM to 10 mM, about 10 mM to 100 mM, or about 100 mM to 1,000 mM and pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pharmaceutical composition comprises benztropine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and pitolisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

M some embodiments, the pharmaceutical composition comprises benztropine and pitolisant. In some embodiments, benztropine at a daily dose of about 0.01 mg to 500 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 25 mg, about 25 mg to 50 mg, about 50 mg to 100 mg, or about 100 mg to 500 mg and pitolisant at a daily dose of about 0.01 mg to 1,000 mg, about 0.1 mg to 100 mg, about 1 mg to 50 mg, about 5 mg to 50 mg, about 5 mg to 10 mg, about 9 mg to 36 mg about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 1,000 mg.

In some embodiments, the pharmaceutical composition comprises an MAChR antagonist/inverse agonist/partial agonist that is clemastine and an H3 antagonist/inverse agonist/partial agonist that is ABT-288 and is administered to a subject. In some embodiments, clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and ABT-288 is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises clemastine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and ABT-288 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises clemastine and ABT-288. In some embodiments, the pharmaceutical composition comprises clemastine and is administered to a subject systemically at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 500 mg and ABT-288 at a daily dose of about 0.01 mg to 1,000 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1,000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 100 mg, about 100 mg to 500 mg, or about 500 mg to 1000 mg.

In some embodiments, the pharmaceutical composition comprises an MAChR antagonist/inverse agonist/partial agonist that is clemastine and an H3 antagonist/inverse agonist/partial agonist that is Bavisant and is administered to a subject. In some embodiments, clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and Bavisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises clemastine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and Bavisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises clemastine and Bavisant. In some embodiments, the pharmaceutical composition comprises clemastine at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 500 mg and Bavisant at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 500 mg, or about 500 mg to 1000 mg.

In some embodiments, the pharmaceutical composition comprises an MAChR antagonist/inverse agonist/partial agonist that is clemastine and an H3 antagonist/inverse agonist/partial agonist that is GSK239512 and is administered to a subject. In some embodiments, clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and GSK239512 is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises clemastine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and GSK239512 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises clemastine and GSK239512. In some embodiments, the pharmaceutical composition comprises clemastine at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 500 mg and GSK239512 at a daily dose of about 0.001 mg to 1,000 mg about 0.01 mg to 100 mg, about 0.1 mg to 10 mg, about 0.001 mg to 0.01 mg, about 0.01 mg to 0.1 mg, about 0.1 mg to 1 mg, about 1 mg to 10 mg, about 10 mg to 100 mg, about 100 mg to 1,000 mg, or about 0.01 mg to 0.08 mg.

In some embodiments, the pharmaceutical composition comprises an MAChR antagonist/inverse agonist/partial agonist that is clemastine and an H3 antagonist/inverse agonist/partial agonist that is Irdabisant and is administered to a subject. In some embodiments, clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises clemastine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and Irdabisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises clemastine and Irdabisant. In some embodiments, the pharmaceutical composition comprises clemastine at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 500 mg and Irdabisant at a daily dose of about 0.001 mg to 1,000 mg, about 0.01 mg to 100 mg, 0.1 mg to 10 mg, about 0.02 mg to 5 mg, about 0.001 mg to 0.01 mg, about 0.01 mg to 0.1 mg, about 0.1 mg to 1 mg, about 1 mg to 10 mg, about 10 mg to 100 mg, or about 100 mg to 1,000 mg.

In some embodiments, the pharmaceutical composition comprises an MAChR antagonist/inverse agonist/partial agonist that is clemastine and an H3 antagonist/inverse agonist/partial agonist that is MK-0249 and is administered to a subject. In some embodiments, clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises clemastine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and MK-0249 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises clemastine and MK-0249. In some embodiments, the pharmaceutical composition comprises clemastine at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 500 mg and MK-0249 at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, or about 50 mg to 100 mg, about 100 mg to 500 mg, or about 500 mg to 1000 mg.

In some embodiments, the pharmaceutical composition comprises an MAChR antagonist/inverse agonist/partial agonist that is clemastine and an H3 antagonist/inverse agonist/partial agonist that is pitolisant and is administered to a subject. In some embodiments, clemastine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and the pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pharmaceutical composition comprises clemastine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and pitolisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the pharmaceutical composition comprises clemastine and pitolisant. In some embodiments, the pharmaceutical composition comprises clemastine at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 500 mg and pitolisant at a daily dose of about 0.01 mg to 1,000 mg, about 0.1 mg to 100 mg, about 1 mg to 50 mg, about 5 mg to 50 mg, about 5 mg to 10 mg, about 9 mg to 36 mg about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 1,000 mg.

In some embodiments, the pharmaceutical composition comprises an MAChR antagonist/inverse agonist/partial agonist that is oxybutynin and an H3 antagonist/inverse agonist/partial agonist that is ABT-288 and is administered to a subject. In some embodiments, oxybutynin and is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and ABT-288 and is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises oxybutynin at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and ABT-288 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises oxybutynin ABT-288. In some embodiments, the pharmaceutical composition comprises oxybutynin at a daily dose of about 0.01 mg to 1,000 mg about 0.1 mg to 100 mg, about 1 mg to 100 mg, about 5 mg to 30 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 20 mg, about 20 mg to 30 mg, about 30 mg to 50 mg, about 50 mg to 100 mg, about 100 mg to 250 mg, or about 250 mg to 500 mg and ABT-288 at a daily dose of about 0.01 mg to 1,000 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1,000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 100 mg, about 100 mg to 500 mg, or about 500 mg to 1000 mg.

In some embodiments, the pharmaceutical composition comprises an MAChR antagonist/inverse agonist/partial agonist that is oxybutynin and an H3 antagonist/inverse agonist/partial agonist that is Bavisant and is administered to a subject. In some embodiments, oxybutynin is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and Bavisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises oxybutynin at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and Bavisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises oxybutynin and Bavisant. In some embodiments, the pharmaceutical composition comprises oxybutynin at a daily dose of about 0.01 mg to 1,000 mg about 0.1 mg to 100 mg, about 1 mg to 100 mg, about 5 mg to 30 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 20 mg, about 20 mg to 30 mg, about 30 mg to 50 mg, about 50 mg to 100 mg, about 100 mg to 250 mg, or about 250 mg to 500 mg and Bavisant at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 500 mg, or about 500 mg to 1000 mg.

In some embodiments, the pharmaceutical composition comprises an MAChR antagonist/inverse agonist/partial agonist that is oxybutynin and an H3 antagonist/inverse agonist/partial agonist that is GSK239512 and is administered to a subject. In some embodiments, oxybutynin is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and GSK239512 is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises oxybutynin at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and GSK239512 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises oxybutynin and GSK239512. In some embodiments, the pharmaceutical composition comprises oxybutynin at a daily dose of about 0.01 mg to 1,000 mg about 0.1 mg to 100 mg, about 1 mg to 100 mg, about 5 mg to 30 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 20 mg, about 20 mg to 30 mg, about 30 mg to 50 mg, about 50 mg to 100 mg, about 100 mg to 250 mg, or about 250 mg to 500 mg and GSK239512 at a daily dose of about 0.001 mg to 1,000 mg about 0.01 mg to 100 mg, about 0.1 mg to 10 mg, about 0.001 mg to 0.01 mg, about 0.01 mg to 0.1 mg, about 0.1 mg to 1 mg, about 1 mg to 10 mg, about 10 mg to 100 mg, about 100 mg to 1,000 mg, or about 0.01 mg to 0.08 mg.

In some embodiments, the pharmaceutical composition comprises an MAChR antagonist/inverse agonist/partial agonist that is oxybutynin and an H3 antagonist/inverse agonist/partial agonist that is Irdabisant and is administered to a subject. In some embodiments, oxybutynin is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises oxybutynin at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and Irdabisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises oxybutynin and the pharmaceutical composition comprises Irdabisant. In some embodiments, the pharmaceutical composition comprises oxybutynin at a daily dose of about 0.01 mg to 1,000 mg about 0.1 mg to 100 mg, about 1 mg to 100 mg, about 5 mg to 30 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 20 mg, about 20 mg to 30 mg, about 30 mg to 50 mg, about 50 mg to 100 mg, about 100 mg to 250 mg, or about 250 mg to 500 mg and Irdabisant at a daily dose of about 0.001 mg to 1,000 mg, about 0.01 mg to 100 mg, 0.1 mg to 10 mg, about 0.02 mg to 5 mg, about 0.001 mg to 0.01 mg, about 0.01 mg to 0.1 mg, about 0.1 mg to 1 mg, about 1 mg to 10 mg, about 10 mg to 100 mg, or about 100 mg to 1,000 mg.

In some embodiments, the pharmaceutical composition comprises a MAChR antagonist/inverse agonist/partial agonist that is oxybutynin and an H3 antagonist/inverse agonist/partial agonist that is MK-0249 and is administered to a subject. In some embodiments, oxybutynin is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises oxybutynin at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and MK-0249 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises oxybutynin and MK-0249. In some embodiments, the pharmaceutical composition comprises oxybutynin at a daily dose of about 0.01 mg to 1,000 mg about 0.1 mg to 100 mg, about 1 mg to 100 mg, about 5 mg to 30 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 20 mg, about 20 mg to 30 mg, about 30 mg to 50 mg, about 50 mg to 100 mg, about 100 mg to 250 mg, or about 250 mg to 500 mg and MK-0249 at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, or about 50 mg to 100 mg, about 100 mg to 500 mg, or about 500 mg to 1000 mg.

In some embodiments, the pharmaceutical composition comprises an MAChR antagonist/inverse agonist/partial agonist that is oxybutynin and an H3 antagonist/inverse agonist/partial agonist that is pitolisant and is administered to a subject. In some embodiments, oxybutynin is administered to a subject, at a concentration of 0.01 μM to 1,000 mM, about 0.1 μM to 100,000 μM, about 0.1 μM to 10,000 μM, about 1 μM to 1,000 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pharmaceutical composition comprises oxybutynin at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and pitolisant is administered to a subject at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the pharmaceutical composition comprises oxybutynin and pitolisant. In some embodiments, the pharmaceutical composition comprises oxybutynin at a daily dose of about 0.01 mg to 1,000 mg about 0.1 mg to 100 mg, about 1 mg to 100 mg, about 5 mg to 30 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, 5 mg to 10 mg, about 10 mg to 20 mg, about 20 mg to 30 mg, about 30 mg to 50 mg, about 50 mg to 100 mg, about 100 mg to 250 mg, or about 250 mg to 500 mg and pitolisant at a daily dose of about 0.01 mg to 1,000 mg, about 0.1 mg to 100 mg, about 1 mg to 50 mg, about 5 mg to 50 mg, about 5 mg to 10 mg, about 9 mg to 36 mg about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 1,000 mg.

In some embodiments, the pharmaceutical composition comprises an MAChR antagonist/inverse agonist/partial agonist that is pentoxyverine and an H3 antagonist/inverse agonist/partial agonist that is ABT-288 and is administered to a subject. In some embodiments, pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and ABT-288 is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises pentoxyverine at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and ABT-288 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises pentoxyverine and ABT-288. In some embodiments, the pharmaceutical composition comprises pentoxyverine at a daily dose of about 0.1 mg to 10,000 mg, about 1 mg to 1,000 mg, about 5 mg to 500 mg, about 10 mg to 250 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 250 mg, about 250 mg to 500 mg, about 500 mg to 1,000 mg or about 1,000 mg to 10,000 mg and ABT-288 at a daily dose of about 0.01 mg to 1,000 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1,000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 100 mg, about 100 mg to 500 mg, or about 500 mg to 1000 mg.

In some embodiments, the pharmaceutical composition comprises an MAChR antagonist/inverse agonist/partial agonist that is pentoxyverine and an H3 antagonist/inverse agonist/partial agonist that is Bavisant and is administered to a subject. In some embodiments, pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and Bavisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises pentoxyverine at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and Bavisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises pentoxyverine and Bavisant. In some embodiments, the pharmaceutical composition comprises pentoxyverine at a daily dose of about 0.1 mg to 10,000 mg, about 1 mg to 1,000 mg, about 5 mg to 500 mg, about 10 mg to 250 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 250 mg, about 250 mg to 500 mg, about 500 mg to 1,000 mg or about 1,000 mg to 10,000 mg and Bavisant at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 500 mg, or about 500 mg to 1000 mg.

In some embodiments, the pharmaceutical composition comprises an MAChR antagonist/inverse agonist/partial agonist that is pentoxyverine and an H3 antagonist/inverse agonist/partial agonist that is GSK239512 and is administered to a subject. In some embodiments, pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and GSK239512 is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises pentoxyverine at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and GSK239512 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises pentoxyverine and GSK239512. In some embodiments, the pharmaceutical composition comprises pentoxyverine at a daily dose of about 0.1 mg to 10,000 mg, about 1 mg to 1,000 mg, about 5 mg to 500 mg, about 10 mg to 250 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 250 mg, about 250 mg to 500 mg, about 500 mg to 1,000 mg or about 1,000 mg to 10,000 mg and GSK239512 at a daily dose of about 0.001 mg to 1,000 mg about 0.01 mg to 100 mg, about 0.1 mg to 10 mg, about 0.001 mg to 0.01 mg, about 0.01 mg to 0.1 mg, about 0.1 mg to 1 mg, about 1 mg to 10 mg, about 10 mg to 100 mg, about 100 mg to 1,000 mg, or about 0.01 mg to 0.08 mg

In some embodiments, the pharmaceutical composition comprises an MAChR antagonist/inverse agonist/partial agonist that is pentoxyverine and an H3 antagonist/inverse agonist/partial agonist that is Irdabisant and is administered to a subject. In some embodiments, pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises pentoxyverine at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and Irdabisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises pentoxyverine and Irdabisant. In some embodiments, the pharmaceutical composition comprises pentoxyverine at a daily dose of about 0.1 mg to 10,000 mg, about 1 mg to 1,000 mg, about 5 mg to 500 mg, about 10 mg to 250 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 250 mg, about 250 mg to 500 mg, about 500 mg to 1,000 mg or about 1,000 mg to 10,000 mg and Irdabisant at a daily dose of about 0.001 mg to 1,000 mg, about 0.01 mg to 100 mg, 0.1 mg to 10 mg, about 0.02 mg to 5 mg, about 0.001 mg to 0.01 mg, about 0.01 mg to 0.1 mg, about 0.1 mg to 1 mg, about 1 mg to 10 mg, about 10 mg to 100 mg, or about 100 mg to 1,000 mg

In some embodiments, the pharmaceutical composition comprises an MAChR antagonist/inverse agonist/partial agonist that is pentoxyverine and an H3 antagonist/inverse agonist/partial agonist that is MK-0249 and is administered to a subject. In some embodiments, pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises pentoxyverine at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and MK-0249 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises pentoxyverine and MK-0249. In some embodiments, the pharmaceutical composition comprises pentoxyverine at a daily dose of about 0.1 mg to 10,000 mg, about 1 mg to 1,000 mg, about 5 mg to 500 mg, about 10 mg to 250 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 250 mg, about 250 mg to 500 mg, about 500 mg to 1,000 mg or about 1,000 mg to 10,000 mg and MK-0249 at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, or about 50 mg to 100 mg, about 100 mg to 500 mg, or about 500 mg to 1000 mg.

In some embodiments, the pharmaceutical composition comprises an MAChR antagonist/inverse agonist/partial agonist that is pentoxyverine and an H3 antagonist/inverse agonist/partial agonist that is pitolisant and is administered to a subject. In some embodiments, pentoxyverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pharmaceutical composition comprises pentoxyverine at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and pitolisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the pharmaceutical composition comprises pentoxyverine and pitolisant. In some embodiments, the pharmaceutical composition comprises pentoxyverine at a daily dose of about 0.1 mg to 10,000 mg, about 1 mg to 1,000 mg, about 5 mg to 500 mg, about 10 mg to 250 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 250 mg, about 250 mg to 500 mg, about 500 mg to 1,000 mg or about 1,000 mg to 10,000 mg and pitolisant at a daily dose of about 0.01 mg to 1,000 mg, about 0.1 mg to 100 mg, about 1 mg to 50 mg, about 5 mg to 50 mg, about 5 mg to 10 mg, about 9 mg to 36 mg about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 1,000 mg.

In some embodiments, the pharmaceutical composition comprises an MAChR antagonist/inverse agonist/partial agonist that is propiverine and an H3 antagonist/inverse agonist/partial agonist that is ABT-288 and is administered to a subject. In some embodiments, propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and ABT-288 is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises propiverine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and ABT-288 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the pharmaceutical composition comprises propiverine ABT-288. In some embodiments, the pharmaceutical composition comprises propiverine at a daily dose of about 0.01 mg to 1000 mg, about 0.1 mg to 100 mg, about 1 mg to 50 mg, about 1 mg to 25 mg, about 1 mg to 10 mg, about 1 mg to 5 mg, about 5 mg to 45 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 to 10 mg, about 10 mg to 25 mg, about 25 mg to 50 mg, about 50 mg to 100 mg, about 100 mg to 250 mg or about 250 mg to 500 mg and ABT-288 at a daily dose of about 0.01 mg to 1,000 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 1,000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 100 mg, about 100 mg to 500 mg, or about 500 mg to 1000 mg.

In some embodiments, the pharmaceutical composition comprises an MAChR antagonist/inverse agonist/partial agonist that is propiverine and an H3 antagonist/inverse agonist/partial agonist that is Bavisant and is administered to a subject. In some embodiments, propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and Bavisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises propiverine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and Bavisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

M some embodiments, the pharmaceutical composition comprises propiverine and Bavisant. In some embodiments, the pharmaceutical composition comprises propiverine at a daily dose of about 0.01 mg to 1000 mg, about 0.1 mg to 100 mg, about 1 mg to 50 mg, about 1 mg to 25 mg, about 1 mg to 10 mg, about 1 mg to 5 mg, about 5 mg to 45 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 to 10 mg, about 10 mg to 25 mg, about 25 mg to 50 mg, about 50 mg to 100 mg, about 100 mg to 250 mg or about 250 mg to 500 mg and Bavisant at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, about 100 mg to 500 mg, or about 500 mg to 1000 mg.

In some embodiments, the pharmaceutical composition comprises an MAChR antagonist/inverse agonist/partial agonist that is propiverine and an H3 antagonist/inverse agonist/partial agonist that is GSK239512 and is administered to a subject. In some embodiments, propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and GSK239512 is administered to a subject, at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises propiverine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and GSK239512 at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

M some embodiments, the pharmaceutical composition comprises propiverine and GSK239512. In some embodiments, the pharmaceutical composition comprises propiverine at a daily dose of about 0.01 mg to 1000 mg, about 0.1 mg to 100 mg, about 1 mg to 50 mg, about 1 mg to 25 mg, about 1 mg to 10 mg, about 1 mg to 5 mg, about 5 mg to 45 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 to 10 mg, about 10 mg to 25 mg, about 25 mg to 50 mg, about 50 mg to 100 mg, about 100 mg to 250 mg or about 250 mg to 500 mg and GSK239512 at a daily dose of about 0.001 mg to 1,000 mg about 0.01 mg to 100 mg, about 0.1 mg to 10 mg, about 0.001 mg to 0.01 mg, about 0.01 mg to 0.1 mg, about 0.1 mg to 1 mg, about 1 mg to 10 mg, about 10 mg to 100 mg, about 100 mg to 1,000 mg, or about 0.01 mg to 0.08 mg.

In some embodiments, the pharmaceutical composition comprises an MAChR antagonist/inverse agonist/partial agonist that is propiverine and an H3 antagonist/inverse agonist/partial agonist that is Irdabisant and is administered to a subject. In some embodiments, propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and Irdabisant is administered to a subject at a concentration of 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably, the pharmaceutical composition comprises propiverine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and Irdabisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises propiverine and Irdabisant. In some embodiments, the pharmaceutical composition comprises propiverine at a daily dose of about 0.01 mg to 1000 mg, about 0.1 mg to 100 mg, about 1 mg to 50 mg, about 1 mg to 25 mg, about 1 mg to 10 mg, about 1 mg to 5 mg, about 5 mg to 45 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 to 10 mg, about 10 mg to 25 mg, about 25 mg to 50 mg, about 50 mg to 100 mg, about 100 mg to 250 mg or about 250 mg to 500 mg and Irdabisant at a daily dose of about 0.001 mg to 1,000 mg, about 0.01 mg to 100 mg, 0.1 mg to 10 mg, about 0.02 mg to 5 mg, about 0.001 mg to 0.01 mg, about 0.01 mg to 0.1 mg, about 0.1 mg to 1 mg, about 1 mg to 10 mg, about 10 mg to 100 mg, or about 100 mg to 1,000 mg.

In some embodiments, the pharmaceutical composition comprises an MAChR antagonist/inverse agonist/partial agonist that is propiverine and an H3 antagonist/inverse agonist/partial agonist that is MK-0249 and is administered to a subject. In some embodiments, propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and MK-0249 is administered to a subject at a concentration of about 0.001 μM to 1,000 mM, about 0.01 μM to 100,000 μM, about 0.1 μM to 10,000 μM, about 1 μM to 1,000 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1,000 mM.

Preferably the pharmaceutical composition comprises propiverine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and MK-0249 t at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises propiverine and MK-0249. In some embodiments, the pharmaceutical composition comprises propiverine at a daily dose of about 0.01 mg to 1000 mg, about 0.1 mg to 100 mg, about 1 mg to 50 mg, about 1 mg to 25 mg, about 1 mg to 10 mg, about 1 mg to 5 mg, about 5 mg to 45 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 to 10 mg, about 10 mg to 25 mg, about 25 mg to 50 mg, about 50 mg to 100 mg, about 100 mg to 250 mg or about 250 mg to 500 mg and MK-0249 at a daily dose of about 0.01 mg to 1,000 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, or about 50 mg to 100 mg, about 100 mg to 500 mg, or about 500 mg to 1000 mg.

In some embodiments, the pharmaceutical composition comprises an MAChR antagonist/inverse agonist/partial agonist that is propiverine and an H3 antagonist/inverse agonist/partial agonist that is pitolisant and is administered to a subject. In some embodiments, propiverine is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM and pitolisant is administered to a subject at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM.

Preferably, the pharmaceutical composition comprises propiverine at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, to 10 mM and pitolisant at a concentration of about 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, or 10 mM.

In some embodiments, the pharmaceutical composition comprises propiverine pitolisant. In some embodiments, the pharmaceutical composition comprises propiverine at a daily dose of about 0.01 mg to 1000 mg, about 0.1 mg to 100 mg, about 1 mg to 50 mg, about 1 mg to 25 mg, about 1 mg to 10 mg, about 1 mg to 5 mg, about 5 mg to 45 mg, about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 to 10 mg, about 10 mg to 25 mg, about 25 mg to 50 mg, about 50 mg to 100 mg, about 100 mg to 250 mg or about 250 mg to 500 mg and pitolisant at a daily dose of about 0.01 mg to 1,000 mg, about 0.1 mg to 100 mg, about 1 mg to 50 mg, about 5 mg to 50 mg, about 5 mg to 10 mg, about 9 mg to 36 mg about 0.01 mg to 0.1 mg, about 0.1 mg to 1 mg, about 1 mg to 10 mg, about 10 mg to 100 mg, about 100 mg to 1000 mg, about 0.5 mg to 1 mg, about 1 mg to 2 mg, about 2 mg to 3 mg, about 3 mg to 4 mg, about 4 mg to 5 mg, about 5 mg to 10 mg, about 10 mg to 25 mg, about 25 mg to 50 mg, about 50 mg to 100 mg, or about 100 mg to 1,000 mg.

The phrase “pharmaceutically-acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically-acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically-acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

To prepare a pharmaceutical composition, an effective or desired amount of one or more agents is mixed with any pharmaceutical carrier(s) or excipient known to those skilled in the art to be suitable for the particular mode of administration. A pharmaceutical carrier may be liquid, semi-liquid or solid. Solutions or suspensions used for administration may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

In some embodiments, wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Certain compositions comprise at least one antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In some embodiments, the composition comprises a buffer. For example, in certain instances, the buffer is physiological saline or phosphate-buffered saline (PBS).

In some embodiments, the composition is at or near physiological pH. For instance, in some embodiments, the composition has a pH of between about 6 and about 8, including all integers, decimals, and ranges in between, for example, about 6 to about 6.5 to about 7 to about 7.5 to about 8. In specific embodiments, the composition has a pH of about 7.4 (±0.2).

In some instances, compositions or formulations include one or more physiologically-acceptable components, including derivatives or prodrugs, solvates, stereoisomers, racemates, or tautomers thereof with any physiologically acceptable carriers, diluents, and/or excipients.

Administration of agents described herein, in pure form or in an appropriate pharmaceutical composition, can be carried out via any of the accepted modes of administration of agents for serving similar utilities. The pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intrathecal, subcutaneous, and topical administration. Preferred modes of administration depend upon the nature of the condition to be treated or prevented. Particular embodiments include administration by IV infusion or subcutaneous injection.

Typical routes of administering these and related pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, and intrasternal injection or infusion techniques. Pharmaceutical compositions according to certain embodiments of the present invention are formulated so as to allow the active ingredients contained therein to be bioavailable upon administration of the composition to a subject. Compositions that will be administered to a subject or patient may take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of one or more agents described herein, for treatment of a disease or condition of interest.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The pharmaceutical compositions may be prepared by methodology well known in the pharmaceutical art. For example, a pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

In some embodiments, a composition disclosed herein is administered to a subject in need thereof once. In some embodiments, a composition disclosed herein is administered to a subject in need thereof more than once. In some embodiments, a first administration of a composition disclosed herein is followed by a second, third, fourth, or fifth administration of a composition disclosed herein.

The number of times a composition is administered to a subject in need thereof depends on the discretion of a medical professional, the disorder, the severity of the disorder, and the subject's response to the formulation. In some embodiments, the composition disclosed herein is administered once to a subject in need thereof with a mild acute condition. In some embodiments, a composition disclosed herein is administered more than once to a subject in need thereof with a moderate or severe acute condition. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

The compositions may be administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

In some embodiments, a compound or composition disclosed herein is administered to a subject in need thereof once. In some embodiments, a compound or composition disclosed herein is administered to a subject in need thereof more than once. In some embodiments, a first administration of a compound or composition disclosed herein is followed by a second, third, fourth, or fifth administration of a compound or composition disclosed herein.

The number of times a compound or composition is administered to a subject in need thereof depends on the discretion of a medical professional, the disorder, the severity of the disorder, and the subject's response to the formulation. In some embodiments, the compound or composition disclosed herein is administered once to a subject in need thereof with a mild acute condition. In some embodiments, a compound or composition disclosed herein is administered more than once to a subject in need thereof with a moderate or severe acute condition. In the case wherein the subject's condition does not improve, upon the doctor's discretion the compound or composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the compound or composition may administered continuously; alternatively, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10% to 100%, including by way of example only 10%, 15%, 20%, 25%, 30%, 35%, 40%, 45%, 50%, 55%, 60%, 65%, 70%, 75%, 80%, 85%, 90%, 95%, and 100%.

Once the subject's myelination has improved, a maintenance dose can be administered, if necessary. Subsequently, the dosage or the frequency of administration, or both, is optionally reduced, as a function of the symptoms, to a level at which the improved disease, disorder or condition is retained. In certain embodiments, subjects require intermittent treatment on a long-term basis upon any recurrence of symptoms.

Certain embodiments include is a pharmaceutical product comprising a sealed packaging and the compound(s) according to the invention in the container. The container size can be optimized to reduce head space in the container after packaging and any head space may be filled with an inert gas such as nitrogen. Furthermore, container material of construction can be chosen to minimize the moisture and oxygen ingress inside the container after packaging.

### EMBODIMENTS

In further embodiments, enumerated as embodiments 1-158 below, the present disclosure includes:

### Embodiment 1

A method of increasing oligodendrocyte precursor cell (OPC) differentiation, comprising contacting an OPC with:
(a) an agent having dual activity as a muscarinic acetylcholine receptor (MAChR) antagonist/inverse agonist/partial agonist and a histamine H3 receptor antagonist/inverse agonist/partial agonist; or
(b) a first agent having activity as a muscarinic acetylcholine receptor (MAChR) antagonist/inverse agonist/partial agonist and a second agent having activity as a histamine H3 receptor antagonist/inverse agonist/partial agonist, wherein the first agent and second agent can occur in any order or simultaneously, thereby increasing OPC differentiation compared to a vehicle control.

### Embodiment 2

A method of producing an expanded population of oligodendrocytes, comprising contacting an oligodendrocyte precursor cell (OPC) with
(a) an agent having dual activity as a muscarinic acetylcholine receptor (MAChR) antagonist/inverse agonist/partial agonist and a histamine H3 receptor antagonist/inverse agonist/partial agonist;
(b) a first agent having activity as a muscarinic acetylcholine receptor (MAChR) antagonist/inverse agonist/partial agonist and a second agent having activity as a histamine H3 receptor antagonist/inverse agonist/partial agonist wherein the first agent and second agent can occur in any order or simultaneously, thereby producing an expanded population of oligodendrocytes compared to a vehicle control.

### Embodiment 3

A method of increasing remyelination in a subject in need thereof, comprising administering to the subject:
(a) an agent having dual activity as a muscarinic acetylcholine receptor (MAChR) antagonist/inverse agonist/partial agonist and a histamine H3 receptor antagonist/inverse agonist/partial agonist;
(b) a first agent having activity as a muscarinic acetylcholine receptor (MAChR) antagonist/inverse agonist/partial agonist and a second agent having activity as a histamine H3 receptor antagonist/inverse agonist/partial agonist wherein the first agent and second agent can occur in any order or simultaneously, wherein administration of (a) or (b) increases remyelination in the subject.

### Embodiment 4

A method of treating a demyelination disease or disorder in a subject in need thereof, comprising administering to the subject:
(a) an agent having dual activity as a muscarinic acetylcholine receptor (MAChR) antagonist/inverse agonist/partial agonist and a histamine H3 receptor antagonist/inverse agonist/partial agonist;
(b) a first agent having activity as muscarinic acetylcholine receptor (MAChR) antagonist/inverse agonist/partial agonist and a second agent having activity as a histamine H3 receptor antagonist/inverse agonist/partial agonist wherein the first agent and second agent can occur in any order or simultaneously, wherein administration of (a) or (b) increases remyelination and/or reduces a sign or symptom of the disease or disorder in the subject.

### Embodiment 5

The method of embodiments 3 and 4, wherein the subject has a demyelination disease.

### Embodiment 6

The method of embodiment 5, wherein the disease is Acute disseminated encephalomyelitis (ADEM); Acute hemorrhagic leukoencephalitis; Acute optic neuritis; Acute transverse myelitis; Adrenoleukodystrophy; Adrenomyeloneuropathy; Alexander Disease; Alzheimer's Disease; aminoacidurias; Amyotrophic Lateral Sclerosis; Anti-MAG peripheral neuropathy; Anti-MOG associated spectrum; Balo concentric sclerosis; Brain injury; CAMFAK Syndrome; Canavan Disease; Carbon monoxide toxicity; Central pontine myelinolysis; Cerebral hypoxia; Cerebral ischemia; Charcot-Marie-Tooth disease; Chronic inflammatory demyelinating polyneuropathy; Chronic relapsing inflammatory optic neuritis (CRION); Chronic traumatic encephalopathy; clinically isolated syndrome (CIS); Congenital Cataract; Copper deficiency associated condition; Delayed Post-Hypoxic Leukoencephalopathy; diffuse cerebral sclerosis of Schilder; diffuse myelinoclastic sclerosis; extrapontine myelinolysis; Gaucher disease; Guillain-Barré syndrome; Hereditary neuropathy; hereditary neuropathy with liability to pressure palsy; HTLV-1-associated myelopathy; Hurler syndrome; Hypomyelination; hypoxic brain injury; Krabbe Disease; Leber hereditary optic atrophy and related mitochondrial disorders; leukodystrophic disorders; Marburg multiple sclerosis; Marchiafava-Bignami disease; Metachromatic leukodystrophy; multiple sclerosis; multiple system atrophy; myelinoclastic disorders; myelopathy; nerve injury; neuromyelitis optica; Neuromyelitis optica (NMO); Niemann-Pick disease; optic neuropathy; optic-spinal multiple sclerosis; Osmotic Demyelination Syndrome; Parkinsons; Pelizaeus-Merzbacher Disease; peripheral neuropathy; Phenylketonuria; primary progressive multiple sclerosis (PPMS); progressive inflammatory neuropathy; progressive multifocal leukoencephalopathy; Progressive subcortical ischemic demyelination; progressive-onset multiple sclerosis; relapsing-onset multiple sclerosis; relapsing-remitting multiple sclerosis (RRMS); reperfusion injury; Schilder disease; secondary progressive multiple sclerosis (SPMS); Solitary sclerosis; Spinal Cord Injury; Subacute sclerosing panencephalitis; Tabes dorsalis; Tay-Sachs disease; Traumatic Brain Injury; Tropical spastic paraparesis; Tumefactive multiple sclerosis; or Vitamin B12 deficiency;

### Embodiment 7

The method of embodiment 5, wherein the disorder is a Multiple Sclerosis, Optic-spinal multiple sclerosis, Amyotrophic Lateral Sclerosis, Chronic relapsing inflammatory optic neuritis (CRION), Neuromyelitis optica, and Chronic inflammatory demyelinating polyneuropathy.

### Embodiment 8

The method of any preceding embodiment, wherein the MAChR antagonist/inverse agonist/partial agonist is Atropine, Benztropine, Chlorpromazine, Clemastine, Dicyclomine, Dip+−* enylpyraline, Disopyramide, Hyoscyamine, Mepenzolate, Orphenadrine, Oxybutynin, Paroxetine, Pentoxyverine, Piperidolate, Propafenone, Propiverine, Quetiapine, Scopolamine, Solifenacin, Tolterodine, Trihexyphenidyl, or Tripelennamine and a histamine H3 receptor antagonist/inverse agonist/partial agonist is A-317920, A-320436, A-331440, ABT-249, ABT-288, ABT-834, AZD-5213, Betahistine, CEP-26401 (Irdabisant), CEP-32215, Ciproxifan, Contilisant, FUB-138, FUB-153, FUB-181, FUB-833, GSK-1004723, GSK-189254, GSK-239512, GSK-247246, GSK-334429, GSK-835726, GT2331, JNJ-31001074/Bavisant, JNJ-39220675, JNJ-5207852, JNJ-6379490, MK-0249, MK-3134, MK-7288, NNC 38-1049, PF-03654746, PF-03654764, Pitolisant (tiprolisant), S 38093, SAR110068, SAR-110894, SUVN G3031, UCL 1390, UW-MD-71, or CAS 1035626-05-1.

### Embodiment 9

The method of any preceding embodiment, wherein the MAChR antagonist/inverse agonist/partial agonist is benztropine, clemastine, oxybutynin, pentoxyverine, or propiverine and a histamine H3 receptor antagonist/inverse agonist/partial agonist is ABT-288, Bavisant, GSK239512, Irdabisant, MK-0249 or pitolisant.

### Embodiment 10

The method of any preceding embodiment, wherein the MAChR antagonist/inverse agonist/partial agonist is benztropine, clemastine, oxybutynin, pentoxyverine, or propiverine.

### Embodiment 11

The method of any preceding embodiment, wherein the MAChR antagonist/inverse agonist/partial agonist is benztropine, Embodiment 12. The method of any preceding embodiment, wherein the MAChR antagonist/inverse agonist/partial agonist is clemastine.

### Embodiment 13

The method of any preceding embodiment, wherein the MAChR antagonist/inverse agonist/partial agonist is oxybutynin.

### Embodiment 14

The method of any preceding embodiment, wherein the MAChR antagonist/inverse agonist/partial agonist is pentoxyverine.

### Embodiment 15

The method of any preceding embodiment, wherein the MAChR antagonist/inverse agonist/partial agonist is propiverine.

### Embodiment 16

The method of any preceding embodiment, wherein the histamine H3 receptor antagonist/inverse agonist/partial agonist is ABT-288, Bavisant, GSK239512, Irdabisant, MK-0249 or pitolisant.

### Embodiment 17

The method of any preceding embodiment, wherein the histamine H3 receptor antagonist/inverse agonist/partial agonist is ABT-288.

### Embodiment 18

The method of any preceding embodiment, wherein the histamine H3 receptor antagonist/inverse agonist/partial agonist is Bavisant, Embodiment 19. The method of any preceding embodiment, wherein the histamine H3 receptor antagonist/inverse agonist/partial agonist is GSK239512.

### Embodiment 20

The method of any preceding embodiment, wherein the histamine H3 receptor antagonist/inverse agonist/partial agonist is Irdabisant.

### Embodiment 21

The method of any preceding embodiment, wherein the histamine H3 receptor antagonist/inverse agonist/partial agonist is MK-0249.

### Embodiment 22

The method of any preceding embodiment, wherein the histamine H3 receptor antagonist/inverse agonist/partial agonist is pitolisant.

### Embodiment 23

The method of any preceding embodiment, wherein the MAChR antagonist/inverse agonist/partial agonist is benztropine and the histamine H3 receptor antagonist/inverse agonist/partial agonist is ABT-288.

### Embodiment 24

The method of any preceding embodiment, wherein the MAChR antagonist/inverse agonist/partial agonist is benztropine and the histamine H3 receptor antagonist/inverse agonist/partial agonist is Bavisant Embodiment 25. The method of any preceding embodiment, wherein the MAChR antagonist/inverse agonist/partial agonist is benztropine and the histamine H3 receptor antagonist/inverse agonist/partial agonist is GSK239512.

### Embodiment 26

The method of any preceding embodiment, wherein the MAChR antagonist/inverse agonist/partial agonist is benztropine and histamine H3 receptor antagonist/inverse agonist/partial agonist is Irdabisant.

### Embodiment 27

The method of any preceding embodiment, wherein the MAChR antagonist/inverse agonist/partial agonist is benztropine and the histamine H3 receptor antagonist/inverse agonist/partial agonist is MK-0249.

### Embodiment 28

The method of any preceding embodiment, wherein the MAChR antagonist/inverse agonist/partial agonist is benztropine and the histamine H3 receptor antagonist/inverse agonist/partial agonist is pitolisant.

### Embodiment 29

The method of any preceding embodiment, wherein the MAChR antagonist/inverse agonist/partial agonist is clemastine and the the histamine H3 receptor antagonist/inverse agonist/partial agonist is ABT-288.

### Embodiment 30

The method of any preceding embodiment, wherein the MAChR antagonist/inverse agonist/partial agonist is clemastine and the the histamine H3 receptor antagonist/inverse agonist/partial agonist is Bavisant.

### Embodiment 31

The method of any preceding embodiment, wherein the MAChR antagonist/inverse agonist/partial agonist is clemastine and the the histamine H3 receptor antagonist/inverse agonist/partial agonist is GSK239512.

### Embodiment 32

The method of any preceding embodiment, wherein the MAChR antagonist/inverse agonist/partial agonist is clemastine and the the histamine H3 receptor antagonist/inverse agonist/partial agonist is Irdabisant.

### Embodiment 33

The method of any preceding embodiment, wherein the MAChR antagonist/inverse agonist/partial agonist is clemastine and the the histamine H3 receptor antagonist/inverse agonist/partial agonist is MK-0249.

### Embodiment 34

The method of any preceding embodiment, wherein the MAChR antagonist/inverse agonist/partial agonist is clemastine and the the histamine H3 receptor antagonist/inverse agonist/partial agonist pitolisant.

### Embodiment 35

The method of any preceding embodiment, wherein the MAChR antagonist/inverse agonist/partial agonist is oxybutynin and the histamine H3 receptor antagonist/inverse agonist/partial agonist is ABT-288.

### Embodiment 36

The method of any preceding embodiment, wherein the MAChR antagonist/inverse agonist/partial agonist is oxybutynin and the histamine H3 receptor antagonist/inverse agonist/partial agonist is Bavisant.

### Embodiment 37

The method of any preceding embodiment, wherein the MAChR antagonist/inverse agonist/partial agonist is oxybutynin and the histamine H3 receptor antagonist/inverse agonist/partial agonist is GSK239512.

### Embodiment 38

The method of any preceding embodiment, wherein the MAChR antagonist/inverse agonist/partial agonist is oxybutynin and the histamine H3 receptor antagonist/inverse agonist/partial agonist is Irdabisant.

### Embodiment 39

The method of any preceding embodiment, wherein the MAChR antagonist/inverse agonist/partial agonist is oxybutynin and the histamine H3 receptor antagonist/inverse agonist/partial agonist is MK-0249.

### Embodiment 40

The method of any preceding embodiment, wherein the MAChR antagonist/inverse agonist/partial agonist is oxybutynin and the histamine H3 receptor antagonist/inverse agonist/partial agonist is pitolisant.

### Embodiment 41

The method of any preceding embodiment, wherein the MAChR antagonist/inverse agonist/partial agonist is pentoxyverine and the histamine H3 receptor antagonist/inverse agonist/partial agonist is ABT-288.

### Embodiment 42

The method of any preceding embodiment, wherein the MAChR antagonist/inverse agonist/partial agonist is pentoxyverine and the histamine H3 receptor antagonist/inverse agonist/partial agonist is Bavisant.

### Embodiment 43

The method of any preceding embodiment, wherein the MAChR antagonist/inverse agonist/partial agonist is pentoxyverine and the histamine H3 receptor antagonist/inverse agonist/partial agonist is GSK239512.

### Embodiment 44

The method of any preceding embodiment, wherein the MAChR antagonist/inverse agonist/partial agonist is pentoxyverine and the histamine H3 receptor antagonist/inverse agonist/partial agonist is Irdabisant.

### Embodiment 45

The method of any preceding embodiment, wherein the MAChR antagonist/inverse agonist/partial agonist is pentoxyverine and the histamine H3 receptor antagonist/inverse agonist/partial agonist is MK-0249.

### Embodiment 46

The method of any preceding embodiment, wherein the MAChR antagonist/inverse agonist/partial agonist is pentoxyverine and the histamine H3 receptor antagonist/inverse agonist/partial agonist is pitolisant.

### Embodiment 47

The method of any preceding embodiment, wherein the MAChR antagonist/inverse agonist/partial agonist is propiverine and the the histamine H3 receptor antagonist/inverse agonist/partial agonist is ABT-288.

### Embodiment 48

The method of any preceding embodiment, wherein the MAChR antagonist/inverse agonist/partial agonist is propiverine and the the histamine H3 receptor antagonist/inverse agonist/partial agonist is Bavisant.

### Embodiment 49

The method of any preceding embodiment, wherein the MAChR antagonist/inverse agonist/partial agonist is propiverine and the the histamine H3 receptor antagonist/inverse agonist/partial agonist is GSK239512.

### Embodiment 50

The method of any preceding embodiment, wherein the MAChR antagonist/inverse agonist/partial agonist is propiverine and the the histamine H3 receptor antagonist/inverse agonist/partial agonist is Irdabisant.

### Embodiment 51

The method of any preceding embodiment, wherein the MAChR antagonist/inverse agonist/partial agonist is propiverine and the the histamine H3 receptor antagonist/inverse agonist/partial agonist is MK-0249.

### Embodiment 52

The method of any preceding embodiment, wherein the MAChR antagonist/inverse agonist/partial agonist is propiverine and the the histamine H3 receptor antagonist/inverse agonist/partial agonist is pitolisant.

### Embodiment 53

The method of any of embodiments 11 and 23-28 wherein benztropine is at a concentration of about between about 100 nM to 10 μM.

### Embodiment 54

The method of any of embodiments 12 and 29-34 wherein clemastine is at a concentration of about between about 25 nM to 2.5 μM.

### Embodiment 55

The method of any of embodiment 13 and 25-40, wherein oxybutynin is at a concentration of about between about 100 nM to 10 μM.

### Embodiment 56

The method of any of embodiments 14 and 41-46, wherein pentoxyverine is at a concentration of about between about 25 nM to 2.5 μM.

### Embodiment 57

The method of any of embodiments 15 and 47-52, wherein propiverine is at a concentration of about between about 100 nM to 10 μM.

### Embodiment 58

The method of any of embodiments 17, 23, 29, 35, 41, and 47, wherein ABT-288 is at a concentration of about between about 10 nM to 1 μM.

### Embodiment 59

The method of any of embodiments 18, 24, 30, 36, 42, and 48, wherein Bavisant is at a concentration of about between about 10 nM to 1 μM.

### Embodiment 60

The method of any of embodiments 19, 25, 31, 37, 43 and 49 wherein GSK239512 is at a concentration of about between about 10 nM to 1 μM.

### Embodiment 61

The method of any of embodiments 20, 26, 32, 38, 44 and 50, wherein Irdabisant is at a concentration of about between about 10 nM to 1 μM.

### Embodiment 62

The method of any of embodiments 21, 27, 33, 39, 95 and 51, wherein MK-0249 is at a concentration of about between about 10 nM to 1 μM.

### Embodiment 63

The method of any of embodiments 22, 29, 34, 40, 46 and 52, wherein pitolisant is at a concentration of about between about 10 nM to 1 μM.

### Embodiment 64

The method of any preceding embodiment, wherein the MAChR antagonist/inverse agonist/partial agonist is administered locally.

### Embodiment 65

The method of any preceding embodiment, wherein the MAChR antagonist/inverse agonist/partial agonist is administered systemically.

### Embodiment 66

The method of any preceding embodiment, wherein the MAChR antagonist/inverse agonist/partial agonist is administered locally and systemically.

### Embodiment 67

The method of any preceding embodiment, wherein the histamine H3 receptor antagonist/inverse agonist/partial agonist is administered locally.

### Embodiment 68

The method of any preceding embodiment, wherein the histamine H3 receptor antagonist/inverse agonist/partial agonist is administered systemically.

### Embodiment 69

The method of any preceding embodiment, wherein the histamine H3 receptor antagonist/inverse agonist/partial agonist is administered locally and systemically.

### Embodiment 70

The method of any of embodiments 64, 66, 67 and 69, wherein the local administration is to the CNS via intrathecal such as intracerebroventricular (ICV) or to the eye via intraocular injection or eye drops.

### Embodiment 71

The method of embodiment 70, wherein the local administration is to CNS via intracerebroventricular (ICV).

### Embodiment 72

The method of any of embodiments 65, 66, 68 and 69 wherein the systemic administration is oral or parenteral.

### Embodiment 73

The method of embodiment 42, wherein the systemic administration is oral.

### Embodiment 74

The method of embodiment 42, wherein the systemic administration is parental.

### Embodiment 75

The method of any of embodiments 11, 23-38, wherein MAChR antagonist/inverse agonist/partial agonist is benztropine and is administered orally at a dose of 0.1 mg to 100 mg per day.

### Embodiment 76

The method of any of embodiments 12, 28-34, wherein MAChR antagonist/inverse agonist/partial agonist is clemastine and is administered orally at a dose of 0.1 mg to 100 mg per day.

### Embodiment 77

The method of any of embodiments 13, 35-40, wherein MAChR antagonist/inverse agonist/partial agonist is oxybutynin and is administered orally at a dose of 0.5 mg to 500 mg per day.

### Embodiment 78

The method of any of embodiments 14, 41-46, wherein MAChR antagonist/inverse agonist/partial agonist is pentoxyverine, and is administered orally at a dose of 10 mg to 1000 mg per day.

### Embodiment 79

The method of any of embodiments 15, 47-52, wherein MAChR antagonist/inverse agonist/partial agonist is propiverine and is administered orally at a dose of 0.5 mg to 500 mg per day.

### Embodiment 80

The method of any of embodiments 17, 23, 29, 35, 41 and 47, wherein histamine H3 receptor antagonist/inverse agonist/partial agonist is ABT-288 and is administered orally at a dose of 0.25 mg to 250 mg per day.

### Embodiment 81

The method of any of embodiments 18, 24, 30, 36, 42, and 48, wherein histamine H3 receptor antagonist/inverse agonist/partial agonist Bavisant and is administered orally at a dose of 0.1 mg to 500 mg per day.

### Embodiment 82

The method of any of embodiments 19, 25, 31, 37, 43 and 49, wherein histamine H3 receptor antagonist/inverse agonist/partial agonist is GSK239512 and is administered orally at a dose of 0.001 mg to 10 mg per day.

### Embodiment 83

The method of any of embodiments 20, 26, 332, 38, 44 and 50, wherein histamine H3 receptor antagonist/inverse agonist/partial agonist is Irdabisant and is administered orally at a dose of 0.01 mg to 50 mg per day.

### Embodiment 84

The method of any of embodiments 21, 27, 33, 39. 45 and 51, wherein histamine H3 receptor antagonist/inverse agonist/partial agonist is MK-0249 and is administered orally at a dose of 0.1 mg to 100 mg per day.

### Embodiment 85

The method of any of embodiments 22, 29, 34, 40, 46 or 52, wherein histamine H3 receptor antagonist/inverse agonist/partial agonist is pitolisant and is administered orally at a dose of 1 mg to 250 mg per day.

### Embodiment 86

A pharmaceutical composition, comprising a pharmaceutically-acceptable carrier, and
a) an agent having dual activity as a muscarinic acetylcholine receptor (MAChR) antagonist/inverse agonist/partial agonist and a histamine H3 receptor antagonist/inverse agonist/partial agonist; or
(b) a first agent having activity as a muscarinic acetylcholine receptor (MAChR) antagonist/inverse agonist/partial agonist and a second agent having activity as a histamine H3 receptor antagonist/inverse agonist/partial agonist.

### Embodiment 87

The pharmaceutical composition of embodiment 86, wherein the MAChR antagonist/inverse agonist/partial agonist is selected from the group consisting benztropine, clemastine, oxybutynin, pentoxyverine, and propiverine Embodiment 88. The pharmaceutical composition of embodiment 87, wherein the MAChR antagonist/inverse agonist/partial agonist is benztropine.

### Embodiment 89

The pharmaceutical composition of embodiment 87, wherein the MAChR antagonist/inverse agonist/partial agonist is clemastine.

### Embodiment 90

The pharmaceutical composition embodiment 87, wherein the MAChR antagonist/inverse agonist/partial agonist is oxybutynin.

### Embodiment 91

The pharmaceutical composition of embodiment 87, wherein the MAChR antagonist/inverse agonist/partial agonist is pentoxyverine.

### Embodiment 92

The pharmaceutical composition of embodiment 87, wherein the MAChR antagonist/inverse agonist/partial agonist is propiverine.

### Embodiment 93

The pharmaceutical composition of embodiment 86 wherein the histamine H3 receptor antagonist/inverse agonist/partial agonist is ABT-288, Bavisant, GSK239512, Irdabisant, MK-0249 or pitolisant.

### Embodiment 94

The pharmaceutical composition of embodiment 93, wherein the histamine H3 receptor antagonist/inverse agonist/partial agonist is ABT-288.

### Embodiment 95

The pharmaceutical composition of embodiment 93, wherein the histamine H3 receptor antagonist/inverse agonist/partial agonist is Bavisant, Embodiment 96. The pharmaceutical composition of embodiment 93, wherein the histamine H3 receptor antagonist/inverse agonist/partial agonist is GSK239512.

### Embodiment 97

The pharmaceutical composition of embodiment 93, wherein the histamine H3 receptor antagonist/inverse agonist/partial agonist is Irdabisant.

### Embodiment 98

The pharmaceutical composition of embodiment 93, wherein the histamine H3 receptor antagonist/inverse agonist/partial agonist is MK-0249.

### Embodiment 99

The pharmaceutical composition of embodiment 93, wherein the histamine H3 receptor antagonist/inverse agonist/partial agonist is pitolisant.

### Embodiment 100

The pharmaceutical composition of embodiment 86, wherein the MAChR antagonist/inverse agonist/partial agonist is benztropine and the histamine H3 receptor antagonist/inverse agonist/partial agonist is ABT-288.

### Embodiment 101

The pharmaceutical composition of embodiment 86, wherein the MAChR antagonist/inverse agonist/partial agonist is benztropine and the histamine H3 receptor antagonist/inverse agonist/partial agonist is Bavisant Embodiment 102. The pharmaceutical composition of embodiment 86, wherein the MAChR antagonist/inverse agonist/partial agonist is benztropine and the histamine H3 receptor antagonist/inverse agonist/partial agonist is GSK239512.

### Embodiment 103

The pharmaceutical composition of embodiment 86, wherein the MAChR antagonist/inverse agonist/partial agonist is benztropine and histamine H3 receptor antagonist/inverse agonist/partial agonist is Irdabisant.

### Embodiment 104

The pharmaceutical composition of embodiment 86, wherein the MAChR antagonist/inverse agonist/partial agonist is benztropine and the histamine H3 receptor antagonist/inverse agonist/partial agonist is MK-0249.

### Embodiment 105

The pharmaceutical composition of embodiment 86, wherein the MAChR antagonist/inverse agonist/partial agonist is benztropine and the histamine H3 receptor antagonist/inverse agonist/partial agonist is pitolisant.

### Embodiment 106

The pharmaceutical composition of embodiment 86, wherein the MAChR antagonist/inverse agonist/partial agonist is clemastine and the the histamine H3 receptor antagonist/inverse agonist/partial agonist is ABT-288.

### Embodiment 107

The pharmaceutical composition of embodiment 86, wherein the MAChR antagonist/inverse agonist/partial agonist is clemastine and the the histamine H3 receptor antagonist/inverse agonist/partial agonist is Bavisant.

### Embodiment 108

The pharmaceutical composition of embodiment 86, wherein the MAChR antagonist/inverse agonist/partial agonist is clemastine and the the histamine H3 receptor antagonist/inverse agonist/partial agonist is GSK239512.

### Embodiment 109

The pharmaceutical composition of embodiment 86, wherein the MAChR antagonist/inverse agonist/partial agonist is clemastine and the the histamine H3 receptor antagonist/inverse agonist/partial agonist is Irdabisant.

### Embodiment 110

The pharmaceutical composition of embodiment 86, wherein the MAChR antagonist/inverse agonist/partial agonist is clemastine and the the histamine H3 receptor antagonist/inverse agonist/partial agonist is MK-0249.

### Embodiment 111

The pharmaceutical composition of embodiment 86, wherein the MAChR antagonist/inverse agonist/partial agonist is clemastine and the the histamine H3 receptor antagonist/inverse agonist/partial agonist pitolisant.

### Embodiment 112

The pharmaceutical composition of embodiment 86, wherein the MAChR antagonist/inverse agonist/partial agonist is oxybutynin and the histamine H3 receptor antagonist/inverse agonist/partial agonist is ABT-288.

### Embodiment 113

The pharmaceutical composition of embodiment 86, wherein the MAChR antagonist/inverse agonist/partial agonist is oxybutynin and the histamine H3 receptor antagonist/inverse agonist/partial agonist is Bavisant.

### Embodiment 114

The pharmaceutical composition of embodiment 86, wherein the MAChR antagonist/inverse agonist/partial agonist is oxybutynin and the histamine H3 receptor antagonist/inverse agonist/partial agonist is GSK239512.

### Embodiment 115

The pharmaceutical composition of embodiment 86, wherein the MAChR antagonist/inverse agonist/partial agonist is oxybutynin and the histamine H3 receptor antagonist/inverse agonist/partial agonist is Irdabisant.

### Embodiment 116

The pharmaceutical composition of embodiment 86, wherein the MAChR antagonist/inverse agonist/partial agonist is oxybutynin and the histamine H3 receptor antagonist/inverse agonist/partial agonist is MK-0249.

### Embodiment 117

The pharmaceutical composition of embodiment 86, wherein the MAChR antagonist/inverse agonist/partial agonist is oxybutynin and the histamine H3 receptor antagonist/inverse agonist/partial agonist is pitolisant.

### Embodiment 118

The pharmaceutical composition of embodiment 86, wherein the MAChR antagonist/inverse agonist/partial agonist is pentoxyverine and the histamine H3 receptor antagonist/inverse agonist/partial agonist is ABT-288.

### Embodiment 119

The pharmaceutical composition of embodiment 86, wherein the MAChR antagonist/inverse agonist/partial agonist is pentoxyverine and the histamine H3 receptor antagonist/inverse agonist/partial agonist is Bavisant.

### Embodiment 120

The pharmaceutical composition of embodiment 86, wherein the MAChR antagonist/inverse agonist/partial agonist is pentoxyverine and the histamine H3 receptor antagonist/inverse agonist/partial agonist is GSK239512.

### Embodiment 121

The pharmaceutical composition of embodiment 86, wherein the MAChR antagonist/inverse agonist/partial agonist is pentoxyverine and the histamine H3 receptor antagonist/inverse agonist/partial agonist is Irdabisant.

### Embodiment 122

The pharmaceutical composition of embodiment 86, wherein the MAChR antagonist/inverse agonist/partial agonist is pentoxyverine and the histamine H3 receptor antagonist/inverse agonist/partial agonist is MK-0249.

### Embodiment 123

The pharmaceutical composition of embodiment 86, wherein the MAChR antagonist/inverse agonist/partial agonist is pentoxyverine and the histamine H3 receptor antagonist/inverse agonist/partial agonist is pitolisant.

### Embodiment 124

The pharmaceutical composition of embodiment 86, wherein the MAChR antagonist/inverse agonist/partial agonist is propiverine and the the histamine H3 receptor antagonist/inverse agonist/partial agonist is ABT-288.

### Embodiment 125

The pharmaceutical composition of embodiment 86, wherein the MAChR antagonist/inverse agonist/partial agonist is propiverine and the the histamine H3 receptor antagonist/inverse agonist/partial agonist is Bavisant.

### Embodiment 126

The pharmaceutical composition of embodiment 86, wherein the MAChR antagonist/inverse agonist/partial agonist is propiverine and the the histamine H3 receptor antagonist/inverse agonist/partial agonist is GSK239512.

### Embodiment 127

The pharmaceutical composition of embodiment 86, wherein the MAChR antagonist/inverse agonist/partial agonist is propiverine and the the histamine H3 receptor antagonist/inverse agonist/partial agonist is Irdabisant.

### Embodiment 128

The pharmaceutical composition of embodiment 86, the MAChR antagonist/inverse agonist/partial agonist is propiverine and the the histamine H3 receptor antagonist/inverse agonist/partial agonist is MK-0249.

### Embodiment 129

The pharmaceutical composition of embodiment 86, wherein the MAChR antagonist/inverse agonist/partial agonist is propiverine and the the histamine H3 receptor antagonist/inverse agonist/partial agonist is pitolisant.

### Embodiment 130

The pharmaceutical composition of any of embodiments 88, 100-105, wherein benztropine is at a concentration of about between 1 uM to 1000 μM.

### Embodiment 131

The pharmaceutical composition of any of embodiments 89, 106-111, wherein clemastine is at a concentration of about between 250 nM to 1000 μM.

### Embodiment 132

The pharmaceutical composition of any of embodiments 90, 112-117, wherein oxybutynin is at a concentration of about between 1 uM to 1000 μM.

### Embodiment 133

The pharmaceutical composition of any of embodiments 91, 118-123, wherein pentoxyverine is at a concentration of about between 1 uM to 1000 μM.

### Embodiment 134

The pharmaceutical composition of any of embodiments 92, 124-129, wherein is propiverine is at a concentration of about between 250 nM to 1000 μM.

### Embodiment 135

The pharmaceutical composition of any of embodiments 94, 100, 106, 112 118, and 124, wherein ABT-288 is at a concentration of about between 100 nM to 100 μM.

### Embodiment 136

The pharmaceutical composition of any of embodiments 95, 101, 107, 113, 119, and 125, wherein Bavisant is at a concentration of about between 100 nM to 100 μM.

### Embodiment 137

The pharmaceutical composition of any of embodiments 96, 102, 108, 114, 120, and 126, wherein GSK239512 is at a concentration of about between 100 nM to 100 μM.

### Embodiment 138

The pharmaceutical composition of any of embodiments 97, 103, 109, 115, 121 and 127, wherein Irdabisant is at a concentration of about between 100 nM to 100 μM.

### Embodiment 139

The pharmaceutical composition of any of embodiments 98, 104, 110, 116, 123 and 128, wherein MK-0249 is at a concentration of about between 100 nM to 100 μM.

### Embodiment 140

The pharmaceutical composition of any of embodiments 99, 105, 111, 117, 124, and 129, wherein pitolisant is at a concentration of about between 100 nM to 100 μM.

### Embodiment 141

A container comprising a MAChR antagonist/inverse agonist/partial agonist and instructions, where those instructions describe the MAChR antagonist/inverse agonist/partial agonist use in treating or preventing a demyelinating disorder in a subject, wherein the instructions require that the subject has been, or will be, administered MAChR antagonist/inverse agonist/partial agonist and a histamine H3 receptor antagonist/inverse agonist/partial agonist.

### Embodiment 142

A container comprising a histamine H3 receptor antagonist/inverse agonist/partial agonist and instructions, where those instructions describe the a histamine H3 receptor antagonist/inverse agonist/partial agonist use in treating or preventing a demyelinating disorder in a subject, wherein the instructions require that the subject has been, or will be, administered MAChR antagonist/inverse agonist/partial agonist and a histamine H3 receptor antagonist/inverse agonist/partial agonist.

### Embodiment 143

The container according to embodiments 141 and 143, wherein the demyelinating disorder is Multiple Sclerosis, Optic-spinal multiple sclerosis, Amyotrophic Lateral Sclerosis, Chronic relapsing inflammatory optic neuritis (CRION), Neuromyelitis optica, and Chronic inflammatory demyelinating polyneuropathy.

### Embodiment 144

Any of the above enumerated embodiments wherein MAChR antagonist/inverse agonist/partial agonist is listed in Table 1.

### Embodiment 145

Any of the above enumerated embodiments wherein the H3 receptor antagonist/inverse agonist/partial agonist listed in Table 2.

### Embodiment 146

Any of the above enumerated embodiments wherein the H3 receptor antagonist/inverse agonist/partial agonist is selective for M1 and M3.

### Embodiment 147

The method of any of the embodiments wherein the method induces myelination of an axon.

### Embodiment 148

The method of any of the embodiments wherein the method induces myelination of an axon, particularly an axon of the central nervous system.

### Embodiment 149

The method of any of the embodiments wherein MAChR antagonist/inverse agonist/partial agonist is clemastine and the is H3 receptor antagonist/inverse agonist/partial agonist is pitolisant Embodiment 150. The method of embodiment 151, wherein the clemastine is administered parentally.

### Embodiment 151

The method of embodiment 151, wherein the clemastine is administered orally.

### Embodiment 152

The method of embodiment 151, wherein the pitolisant is administered parentally.

### Embodiment 153

The method of embodiment 151, wherein the pitolisant is administered orally.

### Embodiment 154

The method of any of embodiments 1-85, 147-153 wherein clemastine is administered in an amount amount sufficient to achieve a concentration of about between about 25 nM to 2.5 μM in the CSF.

### Embodiment 155

The method of any of embodiments 1-85, 147-154 wherein pitolisant is administered in an amount amount sufficient to achieve a concentration at a concentration of about between about 10 nM to 1 μM in the CSF.

### Embodiment 156

The method of any of embodiments 1-85 147-154 wherein clemastine is administered in an amount amount sufficient to achieve a concentration of about between about 25 nM to 2.5 μM in the CSF and the pitolisant is administered in an amount amount sufficient to achieve a concentration at a concentration of about between about 10 nM to 1 μM in the CSF.

### Embodiment 157

The method of embodiment 151, wherein the clemastine and is administered orally at a dose of 0.1 mg to 100 mg per day and the pitolisant orally at a dose of 1 mg to 250 mg per day.

### Embodiment 158

The method of embodiment 150, wherein the clemastine and/or pitolisant is administered to the CNS via intrathecal such as intracerebroventricular (ICV)

### Definitions

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

For the purposes of the present disclosure, the following terms are defined below.

The articles “a” and “an” are used herein to refer to one or to more than one (i.e., to at least one) of the grammatical object of the article. By way of example, “an element” means one element or more than one element.

In this application, the use of “or” includes “and/or” unless stated otherwise. As used in this application, the term “comprise” and variations of the term, such as “comprising” and “comprises,” are not intended to exclude other additives, components, integers or steps. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “about” and “approximately” are used as equivalents. Any numerals used in this disclosure with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration” refers to introducing a substance, for example, an agent into a subject. In certain embodiments “causing to be administered” refers to administration of a second component after a first component has already been administered (e.g., at a different time and/or by a different actor).

An “agonist” or “full agonist” refers to an agent that causes an increase in the expression, levels, and/or activity of a target gene, protein, and/or pathway. In some instances, an agonist directly binds to and activates a target protein. In some instances, an agonist increases the activity of a pathway by binding to and modulating the activity of one or more pathway components, for example, by inhibiting the activity of negative regulator(s) of the pathway, or by activating upstream or downstream regulator(s) of the pathway.

An “antagonist” refers to an agent that binds to a target protein, and which in turn decreases, reduces, or otherwise eliminates its activity, for example, by inhibiting its binding to or by other molecules.

The term “binding” refers to a direct association between two molecules, due to, for example, covalent, electrostatic, hydrophobic, and ionic and/or hydrogen-bond interactions, including interactions such as salt bridges and water bridges.

The terms “competitive antagonist” or “competitive inhibitor” or refer to an agent that blocks or otherwise directly interferes with the active site of an enzyme or receptor.

“Decreasing” or “decreases” refers to decreasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference or control.

“Decreasing” or “decreases” also includes decreasing by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a reference or control.

“Eliminate” means to decrease to a level that is undetectable.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The term “functional inhibition of acid sphingomyelinase” (FIASMA) refers to a compound that inhibit ASM function by, for instance but not limited to, causing the protein to be displaced from the lysosomal membrane allowing the cell to degrade the protein and/or binding directly to acid sphingomyelinase (ASM) inhibiting its function. ASM activity measured in a cell as the degradation of radioactive sphingomyelin to ceramide and phosphorylcholine. FIASMA compounds may not be direct ASM inhibitors.

The term “half maximal effective concentration” or “EC50” refers to the concentration of an agent (for example, an agonist) as described herein at which it induces a response halfway between the baseline and maximum after some specified exposure time. The EC50 of a graded dose response curve therefore represents the concentration of a compound at which 50% of its maximal effect is observed. EC50 also represents the plasma concentration required for obtaining 50% of a maximum effect in vivo. Similarly, the “EC90” refers to the concentration of an agent or composition at which 90% of its maximal effect is observed. The “EC90” can be calculated from the “EC50” and the Hill slope, or it can be determined from the data directly, using routine knowledge in the art.

The term half maximal inhibitory concentration (IC50) is a measure of the effectiveness of an agent (for example, an antagonist/inverse agonist) as described herein at which it inhibits a specific biological or biochemical function. This quantitative measure indicates how much of a particular agent is needed to inhibit a given biological process (or component of a process, i.e. an enzyme, cell, cell receptor or microorganism) by half. The values are typically expressed as molar concentration. The IC50 is commonly used as a measure of antagonist drug potency in pharmacological research. In some instances, the IC50 represents the concentration of an agent that is required for 50% inhibition in an in vitro assay, for example, an in vitro binding assay and/or an in vitro functional assay.

The “half-life” of an agent can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

An “inhibitor” refers to an agent that binds to enzymes and decreases their activity causing a decrease in the expression, levels, and/or activity of a target gene, protein, and/or pathway.

“Increasing” or “increases” refers to increasing by at least 5%, for example, 5, 6, 7, 8, 9, 10, 15, 20, 25, 30, 35, 40, 45, 50, 55, 60, 65, 70, 75, 80, 85, 90, 95, 99, 100, 150, 200, 250, 300, 350, 400, 450, or 500% or more, for example, as compared to the level of a reference.

“Increasing” or “increases” also means increases by at least 1-fold, for example, 1, 1.2, 1.5, 1.7, 2, 3, 4, 5, 6, 7, 8, 9, 10, 15, 20, 30, 40, 50, 60, 70, 80, 90, 100, 200, 500, 1000-fold or more, for example, as compared to the level of a as compared to the level of a reference standard.

An “inverse agonist” refers to agent that binds to the target protein but induces a pharmacological response opposite to that of an agonist. One characteristic of an inverse agonist response is that the target protein has a constitutive (i.e., intrinsic or basal) level activity in the absence of any ligand, and an inverse agonist decreases activity of the target protein below the basal level.

“Isolated” refers to a material that is free to varying degrees from components which normally accompany it as found in its native state. “Isolate” denotes a degree of separation from original source or surroundings.

“Mammal” refers to any mammal including but not limited to human, mouse, rat, sheep, monkey, goat, rabbit, hamster, horse, cow or pig.

The terms “non-competitive antagonist” or “non-competitive inhibitor” refer to an agent that binds to an enzyme or receptor somewhere other than the active site of the enzyme, that is, an allosteric site.

In some embodiments, a “partial agonist” includes an agent that causes an increase in the expression, levels, and/or activity of a target gene, protein, and/or pathway at a level that is lower than the maximum level of a full agonist, for example, about or less than about 5, 6, 7, 8, 9, 10, 15, 20, 25, 30, 35, 40, 45, 50, 55, 60, 65, 70, 75, 80% of the maximum level of a full agonist. In some instances, a partial agonist directly binds to and activates a target protein. In some instances, a partial agonist increases the activity of a pathway by binding to and modulating the activity of one or more pathway components, for example, by inhibiting the activity of negative regulator(s) of the pathway, or by activating upstream or downstream regulator(s) of the pathway. Partial agonists by nature refer to agents that bind to and activate a given receptor, but have only partial efficacy at the receptor compared to a full agonist.

In some instances, however, a “partial agonist” displays antagonist activity. For example, if both a full agonist (e.g., endogenous ligand, for example, endogenous M1 receptor ligand or endogenous histamine H3 receptor ligand) and a partial agonist are present, the partial agonist can act as a competitive antagonist, competing with the full agonist (e.g., endogenous ligand) for receptor occupancy and producing a net decrease in the receptor activation relative to the full agonist alone. In some instances, a partial agonist “reduces” the stimulation or overstimulation (i.e., activity) of a receptor when excess amounts of the full agonist (e.g., endogenous ligand) are present, for example, by about or at least about 5, 6, 7, 8, 9, 10, 15, 20, 25, 30, 35, 40, 45, 50, 55, 60, 65, 70, 75, 80, 85, 90, 95% or more relative to the absence of the partial agonist.

A “protean agonist” refers to an agent that exhibits non-classical behavior and is able to act as an agonist and/or an antagonist depending on the experimental conditions, for example, either in the species and/or the degree of constitutive activity.

“Non-human mammal”, as used herein, refers to any mammal that is not a human.

In certain embodiments, the “purity” of any given agent or compound in a composition may be specifically defined. For instance, certain compositions may comprise an agent that is at least 80, 85, 90, 91, 92, 93, 94, 95, 96, 97, 98, 99, or 100% pure, including all decimals in between, as measured, for example and by no means limiting, by high performance liquid chromatography (HPLC), a well-known form of column chromatography used frequently in biochemistry and analytical chemistry to separate, identify, and quantify compounds.

“Reference” means a standard or control condition (e.g., untreated with a test agent or combination of test agents).

The term “sample” refers to a volume or mass obtained, provided, and/or subjected to analysis. In some embodiments, a sample is or comprises a tissue sample, cell sample, a fluid sample, and the like. In some embodiments, a sample is taken from (or is) a subject (e.g., a human or animal subject). In some embodiments, a tissue sample is or comprises brain, hair (including roots), buccal swabs, blood, saliva, semen, muscle, or from any internal organs, or cancer, precancerous, or tumor cells associated with any one of these. A fluid may be, but is not limited to, urine, blood, ascites, pleural fluid, spinal fluid, and the like. A body tissue can include, but is not limited to, brain, skin, muscle, endometrial, uterine, and cervical tissue or cancer, precancerous, or tumor cells associated with any one of these. In an embodiment, a body tissue is brain tissue or a brain tumor or cancer. Those of ordinary skill in the art will appreciate that, in some embodiments, a “sample” is a “primary sample” in that it is obtained from a source (e.g., a subject); in some embodiments, a “sample” is the result of processing of a primary sample, for example to remove certain potentially contaminating components and/or to isolate or purify certain components of interest.

The term “solubility” refers to the property of an agent described herein to dissolve in a liquid solvent and form a homogeneous solution. Solubility is typically expressed as a concentration, either by mass of solute per unit volume of solvent (g of solute per kg of solvent, g per dL (100 mL), mg/mL, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaP). In specific embodiments, solubility is measured at relatively lower pH (e.g., pH 6.0) and relatively higher salt (e.g., 500 mM NaCl and 10 mM NaP). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/mL at room temperature or at 37° C.

“Subject” includes humans and mammals (e.g., mice, rats, pigs, cats, dogs, and horses). In some embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

By “statistically significant”, it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95% or greater of some given quantity.

“Synergy” or “synergistic effect” is an effect which is greater than the sum of each of the effects taken separately; a greater than additive effect.

“Therapeutic response” refers to improvement of symptoms (whether or not sustained) based on administration of one or more agents or compositions.

As used herein, the terms “therapeutically effective amount”, “therapeutic dose,” or “prophylactically effective amount,” is the amount of an agent needed to elicit the desired biological response following administration.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

“Pharmaceutically-acceptable salt” includes both acid and base addition salts.

“Pharmaceutically-acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, /toluenesulfonic acid, trifluoroacetic acid, undecylenic acid, and the like.

“Pharmaceutically-acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Example organic bases used in certain embodiments include isopropylamine, diethylamine, ethanolamine, trimethylamine, dicyclohexylamine, choline, and caffeine.

### EXAMPLES

### Example 1

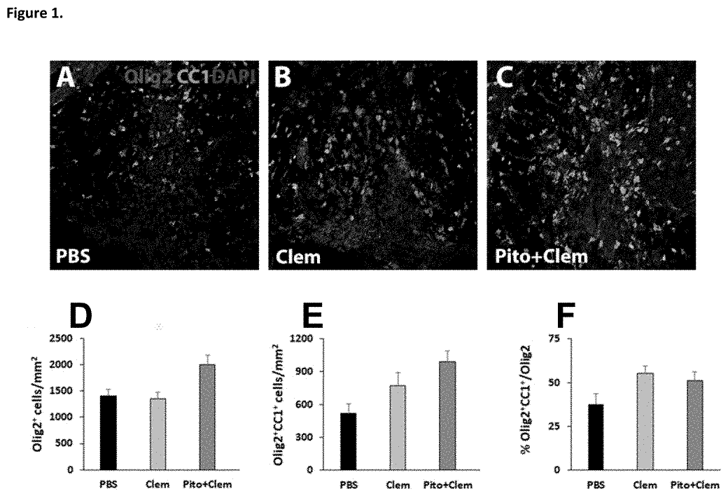

12-month old C57BL/6J female mice (6 per group) were given a focal demyelinating lesion with lysolecithin. After surgical exposure of the spinal cord, a glass capillary needle attached to a 10 μl Hamilton syringe was inserted into the ventral funiculus and 1 μl of 1.0% lysolecithin (Sigma-Aldrich) in PBS was injected to induce a focal demyelinated lesion in the ventral white matter. Mice treated for 7 days starting 3 days post lesion with one of the following three treatments: 1) PBS (vehicle control); 2) 10 mg/kg clemastine (dosed once daily, i.p.); 3) 10 mg/kg clemastine (dosed once daily, i.p.) plus 40 mg/kg pitolisant (dosed twice daily, i.p.). At 10 days post lesion, mice were perfused intracardially with 4% (w/v) paraformaldehyde (PFA; Sigma-Aldrich) in PBS. Dissected spinal cords were post fixed in 4% PFA for 30 min at 4° C., followed by overnight immersion in 30% sucrose in 1×PBS. Spinal cords were then embedded in OCT compound, frozen on the surface of dry ice, and stored at −80° C. Immunohistochemistry was performed to measure the number of mature oligodendrocytes (double labeled positive for both CC1 and Olig2) per mm2, the number of oligodendrocyte lineage cells (labeled positive for Olig2) per mm2, and the percentage of oligodendrocyte lineage cells that are mature (the number of double labeled positive for both CC1 and Olig2 divided by the number of cells labeled positive for Olig2). Cells were also stained with the nuclear dye 4′,6-diamidino-2-phenylindole (DAPI). FIG. 1 shows that the clemastine increases the number of mature oligodendrocytes per mm2 as well as the percentage of mature oligodendrocytes relative to vehicle treated mice, consistent with published data suggesting that clemastine drives the differentiation of oligodendrocyte progenitor cells (OPCs). The combination of pitolisant plus clemastine resulted in more oligodendrocyte lineage cells per mm2 and more mature oligodendrocytes per mm2 relative to clemastine alone, but not an increase in the % of mature oligodendrocyte cells, suggesting that the combination may increase the proliferation of OPCs (as mature oligodendrocytes are unlikely to proliferate under these conditions). Thus, the combination of clemastine and pitolisant may increase both proliferation and differentiation of OPCs relative to vehicle treated mice.

### Example 2

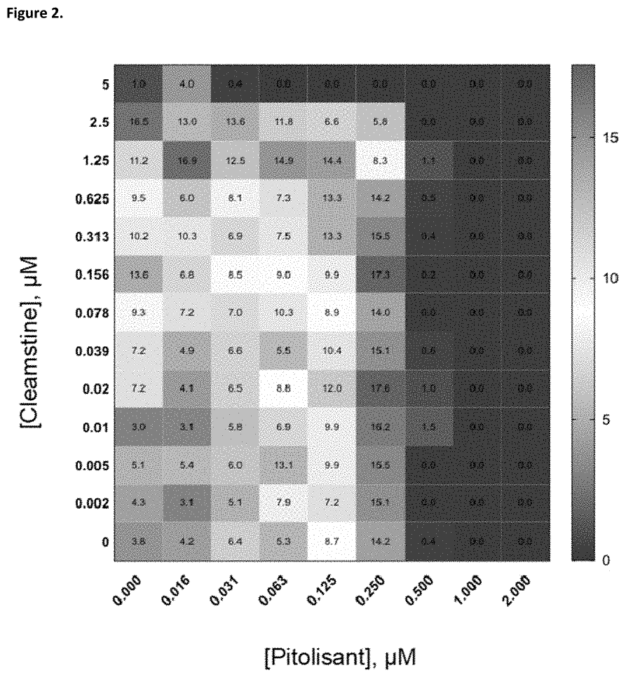

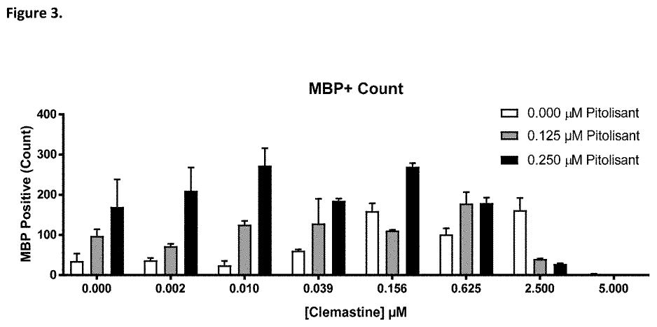

Rat primary optic nerve oligodendrocyte progenitor cells (OPCs) were isolated and cultured, as described in Deshmukh et al. 2013 (Nature 502(7471):327-332. doi: 10.1038/nature12647). OPCs were plated at a density of 1,000 cells per well on poly-D-lysine coated 384-well plates in differentiation media. Compounds were added within 12 h after plating the cells and incubated at 37° C. with 5% CO2 for 6 days. Cells were then fixed, stained for myelin basic protein (MBP) and the nuclear dye 4′,6-diamidino-2-phenylindole (DAPI) and MBP-positive cell bodies were detected using an algorithm that selects for positive cell bodies and nuclei within a range of fluorescent emission values and sizes as determined by fitting parameters to positive (thyroid hormone (T3), 1 mM) and negative controls (DMSO, 0.1%). In this example, cells were treated with a combination of clemastine fumarate (ranging from 0 to 5 μM) and pitolisant hydrochloride (ranging from 0 to 2 μM). The % of MBP+ cells are represented in a heatmap in FIG. 2. Pitolisant alone was found to induce OPC differentiation, with comparable levels of efficacy as that observed for clemastine. Cell death was observed at the highest concentrations of clemastine or pitolisant when the two drugs were combined in this in vitro study. Importantly, pitolisant appears to have dose sparing activity with respect to clemastine. That is, when pitolisant is present at optimal concentrations, the concentration of clemastine needed to achieve a maximal effect is reduced. Similarly, clemastine appears to have dose sparing activity with respect to pitolisant. Furthermore, the combination of clemastine with pitolisant can lead to a greater number of mature, myelinating oligodendrocytes (MBP+ cells) than what can be achieved with either agent alone, as shown in FIG. 3.

### Other Embodiments

Each embodiment in this specification is to be applied to every other embodiment unless expressly stated otherwise.

The various embodiments described herein can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

1. A method of increasing oligodendrocyte precursor cell (OPC) differentiation, comprising contacting an OPC with:
(a) an agent having dual activity as a muscarinic acetylcholine receptor (MAChR) antagonist/inverse agonist/partial agonist and a histamine H3 receptor antagonist/inverse agonist/partial agonist; or
(b) a first agent having activity as a muscarinic acetylcholine receptor (MAChR) antagonist/inverse agonist/partial agonist and a second agent having activity as a histamine H3 receptor antagonist/inverse agonist/partial agonist, wherein the OPC is contacted with the first agent and second agent in any order or simultaneously,
thereby increasing OPC differentiation compared to a vehicle control.

2. A method of producing an expanded population of oligodendrocytes, comprising contacting an oligodendrocyte precursor cell (OPC) with
(a) an agent having dual activity as a muscarinic acetylcholine receptor (MAChR) antagonist/inverse agonist/partial agonist and a histamine H3 receptor antagonist/inverse agonist/partial agonist; or
(b) a first agent having activity as a muscarinic acetylcholine receptor (MAChR) antagonist/inverse agonist/partial agonist and a second agent having activity as a histamine H3 receptor antagonist/inverse agonist/partial agonist, wherein the OPC is contacted with the first agent and second agent in any order or simultaneously,
thereby producing an expanded population of oligodendrocytes compared to a vehicle control.

3. A method of increasing remyelination in a subject in need thereof, comprising administering to the subject:
(a) an agent having dual activity as a muscarinic acetylcholine receptor (MAChR) antagonist/inverse agonist/partial agonist and a histamine H3 receptor antagonist/inverse agonist/partial agonist; or
(b) a first agent having activity as a muscarinic acetylcholine receptor (MAChR) antagonist/inverse agonist/partial agonist and a second agent having activity as a histamine H3 receptor antagonist/inverse agonist/partial wherein administration of the first agent and second agent can occur in any order or simultaneously,
wherein administration of the agent(s) of (a) or (b) increases remyelination in the subject.

4. A method of treating a demyelination disease or disorder in a subject in need thereof, comprising administering to the subject:
(a) an agent having dual activity as a muscarinic acetylcholine receptor (MAChR) antagonist/inverse agonist/partial agonist and a histamine H3 receptor antagonist/inverse agonist/partial agonist; or
(b) a first agent having activity as muscarinic acetylcholine receptor (MAChR) antagonist/inverse agonist/partial agonist and a second agent having activity as a histamine H3 receptor antagonist/inverse agonist/partial agonist wherein administration of the first agent and second agent can occur in any order or simultaneously,
wherein administration of the agent(s) of (a) or (b) increases remyelination and/or reduces a sign or symptom of the disease or disorder in the subject.

5. The method of claim 3, wherein the subject has a demyelination disease.

6. The method of claim 5, wherein the disease is Acute disseminated encephalomyelitis (ADEM); Acute hemorrhagic leukoencephalitis; Acute optic neuritis; Acute transverse myelitis; Adrenoleukodystrophy; Adrenomyeloneuropathy; Alexander Disease; Alzheimer's Disease; aminoacidurias; Amyotrophic Lateral Sclerosis; Anti-MAG peripheral neuropathy; Anti-MOG associated spectrum; Balo concentric sclerosis; Brain injury; CAMFAK Syndrome; Canavan Disease; Carbon monoxide toxicity; Central pontine myelinolysis; Cerebral hypoxia; Cerebral ischemia; Charcot-Marie-Tooth disease; Chronic inflammatory demyelinating polyneuropathy; Chronic relapsing inflammatory optic neuritis (CRION); Chronic traumatic encephalopathy; clinically isolated syndrome (CIS); Congenital Cataract; Copper deficiency associated condition; Delayed Post-Hypoxic Leukoencephalopathy; diffuse cerebral sclerosis of Schilder; diffuse myelinoclastic sclerosis; extrapontine myelinolysis; Gaucher disease; Guillain-Barré syndrome; Hereditary neuropathy; hereditary neuropathy with liability to pressure palsy; HTLV-1-associated myelopathy; Hurler syndrome; Hypomyelination; hypoxic brain injury; Krabbe Disease; Leber hereditary optic atrophy and related mitochondrial disorders; leukodystrophic disorders; Marburg multiple sclerosis; Marchiafava-Bignami disease; Metachromatic leukodystrophy; multiple sclerosis; multiple system atrophy; myelinoclastic disorders; myelopathy; nerve injury; neuromyelitis optica; Neuromyelitis optica (NMO); Niemann-Pick disease; optic neuropathy; optic-spinal multiple sclerosis; Osmotic Demyelination Syndrome; Parkinsons; Pelizaeus-Merzbacher Disease; peripheral neuropathy; Phenylketonuria; primary progressive multiple sclerosis (PPMS); progressive inflammatory neuropathy; progressive multifocal leukoencephalopathy; Progressive subcortical ischemic demyelination; progressive-onset multiple sclerosis; relapsing-onset multiple sclerosis; relapsing-remitting multiple sclerosis (RRMS); reperfusion injury; Schilder disease; secondary progressive multiple sclerosis (SPMS); Solitary sclerosis; Spinal Cord Injury; Subacute sclerosing panencephalitis; Tabes dorsalis; Tay-Sachs disease; Traumatic Brain Injury; Tropical spastic paraparesis; Tumefactive multiple sclerosis; or Vitamin B12 deficiency.

7. (canceled)

8. The method of claim 1, wherein the MAChR antagonist/inverse agonist/partial agonist is Atropine, Benztropine, Chlorpromazine, Clemastine, Dicyclomine, Diphenylpyraline, Disopyramide, Hyoscyamine, Mepenzolate, Orphenadrine, Oxybutynin, Paroxetine, Pentoxyverine, Piperidolate, Propafenone, Propiverine, Quetiapine, Scopolamine, Solifenacin, Tolterodine, Trihexyphenidyl, or Tripelennamine and a histamine H3 receptor antagonist/inverse agonist/partial agonist is A-317920, A-320436, A-331440, ABT-249, ABT-288, ABT-834, AZD-5213, Betahistine, CEP-26401 (Irdabisant), CEP-32215, Ciproxifan, Contilisant, FUB-138, FUB-153, FUB-181, FUB-833, GSK-1004723, GSK-189254, GSK-239512, GSK-247246, GSK-334429, GSK-835726, GT2331, JNJ-31001074/Bavisant, JNJ-39220675, JNJ-5207852, JNJ-6379490, MK-0249, MK-3134, MK-7288, NNC 38-1049, PF-03654746, PF-03654764, Pitolisant (tiprolisant), S 38093, SAR110068, SAR-110894, SUVN G3031, UCL 1390, UW-MD-71, or CAS 1035626-05-1.

9-15. (canceled)

16. The method of claim 1, wherein the histamine H3 receptor antagonist/inverse agonist/partial agonist is ABT-288, Bavisant, GSK239512, Irdabisant, MK-0249 or pitolisant.

17-52. (canceled)

53. The method of claim 8, wherein benztropine is at a concentration of about between about 100 nM to 10 μM.

54. The method of claim 8, wherein clemastine is at a concentration of about between about 25 nM to 2.5 μM.

55. The method of claim 8, wherein oxybutynin is at a concentration of about between about 100 nM to 10 μM.

56. The method of claim 8, wherein pentoxyverine is at a concentration of about between about 25 nM to 2.5 μM.

57. The method of claim 8, wherein propiverine is at a concentration of about between about 100 nM to 10 μM.

58. The method of claim 16, wherein ABT-288 is at a concentration of about between about 10 nM to 1 μM.

59. The method of claim 16, wherein Bavisant is at a concentration of about between about 10 nM to 1 μM.

60. The method of claim 16, wherein GSK239512 is at a concentration of about between about 10 nM to 1 μM.

61. The method of claim 16, wherein Irdabisant is at a concentration of about between about 10 nM to 1 μM.

62. The method of claim 16, wherein MK-0249 is at a concentration of about between about 10 nM to 1 μM.

63. The method of claim 16, wherein pitolisant is at a concentration of about between about 10 nM to 1 μM.

64. The method of claim 1, wherein the MAChR antagonist/inverse agonist/partial agonist is administered locally.

65. The method of claim 1, wherein the MAChR antagonist/inverse agonist/partial agonist is administered systemically.

66. The method of claim 1, wherein the MAChR antagonist/inverse agonist/partial agonist is administered locally and systemically.

67. The method of claim 1, wherein the histamine H3 receptor antagonist/inverse agonist/partial agonist is administered locally.

68. The method of claim 1, wherein the histamine H3 receptor antagonist/inverse agonist/partial agonist is administered systemically.

69. The method of claim 1, wherein the histamine H3 receptor antagonist/inverse agonist/partial agonist is administered locally and systemically.

70. The method of claim 64, wherein the local administration is to the CNS via intrathecal such as intracerebroventricular (ICV) or to the eye via intraocular injection or eye drops.

71. (canceled)

72. The method of claim 65, wherein the systemic administration is oral or parenteral.

73-74. (canceled)

75. The method of claim 8, wherein MAChR antagonist/inverse agonist/partial agonist is benztropine and is administered orally at a dose of 0.1 mg to 100 mg per day.

76. The method of claim 8, wherein MAChR antagonist/inverse agonist/partial agonist is clemastine and is administered orally at a dose of 0.1 mg to 100 mg per day.

77. The method of claim 8, wherein MAChR antagonist/inverse agonist/partial agonist is oxybutynin and is administered orally at a dose of 0.5 mg to 500 mg per day.

78. The method of claim 8, wherein MAChR antagonist/inverse agonist/partial agonist is pentoxyverine, and is administered orally at a dose of 10 mg to 1000 mg per day.

79. The method of claim 8, wherein MAChR antagonist/inverse agonist/partial agonist is propiverine and is administered orally at a dose of 0.5 mg to 500 mg per day.

80. The method of claim 16, wherein histamine H3 receptor antagonist/inverse agonist/partial agonist is ABT-288 and is administered orally at a dose of 0.25 mg to 250 mg per day.

81. The method of claim 16, wherein histamine H3 receptor antagonist/inverse agonist/partial agonist Bavisant and is administered orally at a dose of 0.1 mg to 500 mg per day.

82. The method of claim 16, wherein histamine H3 receptor antagonist/inverse agonist/partial agonist is GSK239512 and is administered orally at a dose of 0.001 mg to 10 mg per day.

83. The method of claim 16, wherein histamine H3 receptor antagonist/inverse agonist/partial agonist is Irdabisant and is administered orally at a dose of 0.01 mg to 50 mg per day.

84. The method of claim 16, wherein histamine H3 receptor antagonist/inverse agonist/partial agonist is MK-0249 and is administered orally at a dose of 0.1 mg to 100 mg per day.

85. The method of claim 16, wherein histamine H3 receptor antagonist/inverse agonist/partial agonist is pitolisant and is administered orally at a dose of 1 mg to 250 mg per day.

86. A pharmaceutical composition, comprising a pharmaceutically-acceptable carrier, and
a) an agent having dual activity as a muscarinic acetylcholine receptor (MAChR) antagonist/inverse agonist/partial agonist and a histamine H3 receptor antagonist/inverse agonist/partial agonist; or
(b) a first agent having activity as a muscarinic acetylcholine receptor (MAChR) antagonist/inverse agonist/partial agonist and a second agent having activity as a histamine H3 receptor antagonist/inverse agonist/partial agonist.

87. The pharmaceutical composition of claim 86, wherein the MAChR antagonist/inverse agonist/partial agonist is selected from the group consisting benztropine, clemastine, oxybutynin, pentoxyverine, and propiverine

88-92. (canceled)

93. The pharmaceutical composition of claim 86 wherein the histamine H3 receptor antagonist/inverse agonist/partial agonist is ABT-288, Bavisant, GSK239512, Irdabisant, MK-0249 or pitolisant.

94-129. (canceled)

130. The pharmaceutical composition of claim 87, wherein benztropine is at a concentration of about between 1 uM to 1000 μM.

131. The pharmaceutical composition of claim 87, wherein clemastine is at a concentration of about between 250 nM to 1000 μM.

132. The pharmaceutical composition of claim 87, wherein oxybutynin is at a concentration of about between 1 uM to 1000 μM.

133. The pharmaceutical composition of claim 87, wherein pentoxyverine is at a concentration of about between 1 uM to 1000 μM.

134. The pharmaceutical composition of claim 87, wherein is propiverine is at a concentration of about between 250 nM to 1000 μM.

135. The pharmaceutical composition of claim 93, wherein ABT-288 is at a concentration of about between 100 nM to 100 μM.

136. The pharmaceutical composition of claim 93, wherein Bavisant is at a concentration of about between 100 nM to 100 μM.

137. The pharmaceutical composition of claim 93, wherein GSK239512 is at a concentration of about between 100 nM to 100 μM.

138. The pharmaceutical composition of claim 93, wherein Irdabisant is at a concentration of about between 100 nM to 100 μM.

139. The pharmaceutical composition of claim 93, wherein MK-0249 is at a concentration of about between 100 nM to 100 μM.

140. The pharmaceutical composition of claim 93, wherein pitolisant is at a concentration of about between 100 nM to 100 μM.

141. A container comprising a MAChR antagonist/inverse agonist/partial agonist and instructions, where those instructions describe the MAChR antagonist/inverse agonist/partial agonist use in treating or preventing a demyelinating disorder in a subject, wherein the instructions require that the subject has been, or will be, administered MAChR antagonist/inverse agonist/partial agonist and a histamine H3 receptor antagonist/inverse agonist/partial agonist.

142. A container comprising a histamine H3 receptor antagonist/inverse agonist/partial agonist and instructions, where those instructions describe the a histamine H3 receptor antagonist/inverse agonist/partial agonist use in treating or preventing a demyelinating disorder in a subject, wherein the instructions require that the subject has been, or will be, administered MAChR antagonist/inverse agonist/partial agonist and a histamine H3 receptor antagonist/inverse agonist/partial agonist.

143. The container according to claim 141, wherein the demyelinating disorder is Multiple Sclerosis, Optic-spinal multiple sclerosis, Amyotrophic Lateral Sclerosis, Chronic relapsing inflammatory optic neuritis (CRION), Neuromyelitis optica, and Chronic inflammatory demyelinating polyneuropathy.
